(19) United States
(12) Patent Application Publication
(10) Pub. No.: US 2002/0155172 A1
(43) Pub. Date: Oct. 24, 2002

(54) Methods and compounds for decreasing cell toxicity or death

(76) Inventors: Junying Yuan, Newton, MA (US); Ivelisse Sanchez, Watertown, MA (US)

Correspondence Address: CLARK & ELBING LLP, 101 FEDERAL STREET, BOSTON, MA, 02110, US

(21) Appl. No.: 09/829,040

(22) Filed: Apr. 9, 2001

Related U.S. Application Data
(60) Provisional application No. 60/195,661, filed on Apr. 7, 2000.

(51) Int. Cl. A61K 31/704; A61K 31/715; A61K 31/655; A61K 38/13

(52) U.S. Cl. 424/692; 514/150; 514/9; 514/179; 514/54; 514/34; 514/26; 514/250; 514/317; 424/725

(57) ABSTRACT

The invention features methods for decreasing cell toxicity or death, and for decreasing polyglutamine aggregates and other amyloidogenic aggregates. The invention also features methods for treating a subject with a condition in which expanded polyglutamine repeats or amyloidogenic aggregates are present.

### CROSS REFERENCE TO RELATED APPLICATIONS

This application claims priority from U.S. Provisional Application Serial No. 60/195,661, filed Apr. 7, 2000.

### BACKGROUND OF THE INVENTION

In general, the invention relates to methods and compounds for decreasing cell death.

A growing number of disorders, such as neurodegenerative diseases including Huntington's disease, spinobulbar muscular atrophy (SBMA), spino-cerebellar ataxia types 1, 2, 6, 7, and 3 (Machado-Joseph disease), dentatorubral-pallidoluysian atrophy, familial schizophrenia, and infertility have been found to be caused by expanded CAG nucleotide triplet repeats (expanded polyglutamine repeats) which code for multiple glutamines. It is thought that the expanded polyglutamine repeats result in toxicity in specific cells, and this toxicity results in cell death. The mechanisms by which cell toxicity and death occur are not well understood, although it is known that in neurodegenerative diseases characterized by polyglutamine repeats, the polyglutamine repeats commonly form aggregates or inclusions.

One way to treat disorders characterized by amyloidogenic protein aggregates, for example, expanded polyglutamine repeats, is to prevent the formation of the aggregates, or break down preformed aggregates. The decrease in the presence of such aggregates should prevent cell toxicity and death.

### SUMMARY OF THE INVENTION

We have discovered methods for preventing polyglutamine repeat formation and disrupting polyglutamine repeats that have formed. These methods may be used to prevent or treat diseases associated with polyglutamine repeats and other amyloidogenic protein aggregates. Our invention also features methods and compounds for decreasing or preventing cell death or toxicity, and for treating conditions in subjects with, or at risk for having, expanded polyglutamine repeats or aggregates, or inclusions formed by amyloidogenic proteins.

Accordingly, in a first aspect the invention features a method for decreasing cell death or toxicity with diphenyldiazo-bis-alpha-napthylaminesulfonate (Congo red), or a pharmaceutically effective derivative or salt thereof, by contacting a cell or animal expressing an expanded polyglutamine repeat with Congo red. In one embodiment, the compounds of the invention are provided in a dose sufficient to decrease or prevent polyglutamine aggregates or inclusions that exist, or might be formed. In another embodiment, cell toxicity is decreased. In a desirable embodiment, both aggregation and toxicity are decreased.

In a second aspect, the invention features a method for decreasing aggregates or inclusions formed by expanded polyglutamine repeats in a cell or animal using Congo red, or a pharmaceutically effective derivative or salt thereof, by contacting a cell or animal expressing an expanded polyglutamine repeat with Congo red or its derivative. In one embodiment, the compounds of the invention are provided in doses sufficient to decrease or prevent polyglutamine aggregates or inclusions that exist, or might be formed. In another embodiment, cell toxicity is decreased. In a desirable embodiment, both aggregation and toxicity are decreased.

In another desirable embodiment of the above aspects of the invention, the expanded polyglutamine repeat is one which is resistant to disruption by at least one of the following compounds: iota-carrageenan, dextran, minocycline, daunomycin, rolitetracycline, or Chrysamine G; or would be, if allowed to form.

In a third aspect, the invention features a method for decreasing cell death or toxicity, involving contacting a cell or an animal expressing an amyloidogenic protein with any of the following: bromocriptine mesylate; haloperidol; nabumetone; primidone; hydrocortisone; phenazopyridine; R-(−)-deprenyl hydrochloride; 6a-methylprednisolone 21-hemisuccinate; digoxin; azathioprine; D-cycloserine; red clover; magnesium oxide; N-vanillylnonanmide; neostigmine methyl ether; or a pharmaceutically effective derivative, salt, or isomer thereof; or a compound, including isomers and salts having the formula selected from any of:

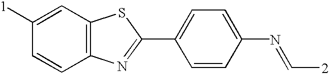

wherein 1 is CH₃ or H, and 2 is

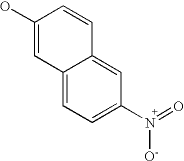

or wherein 1 is CH₃, and 2 is

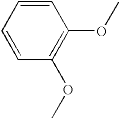

or wherein 1 is CH₃, and 2 is

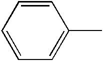

or a pharmaceutically effective derivative, salt, or isomer thereof;

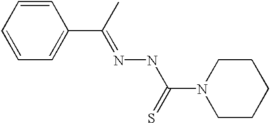

or a pharmaceutically effective derivative, salt, or isomer thereof;

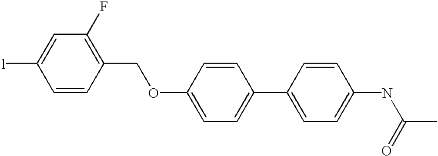

wherein 1 is H or NO₂⁻, or a pharmaceutically effective derivative, salt, or isomer thereof;

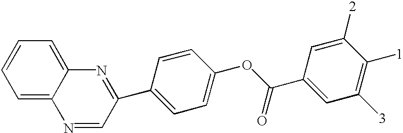

wherein 1 is Cl, and 2 and 3 are H; or wherein 1 and 3 are H, and 2 is NO₂; or wherein 1 is Br, 2 is H, and 3 is NO₂; or wherein 1 is Cl, 2 is H, and 3 is Br, or a pharmaceutically effective derivative, salt, or isomer thereof;

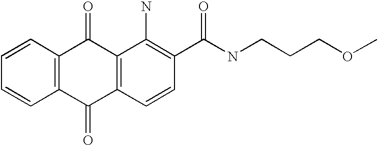

or a pharmaceutically effective derivative, salt, or isomer thereof;

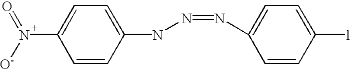

wherein 1 is NO₂, Br, or O₂, or a pharmaceutically effective derivative, salt, or isomer thereof;

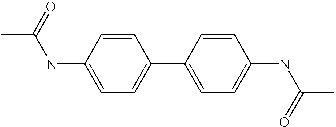

or a pharmaceutically effective derivative, salt, or isomer thereof

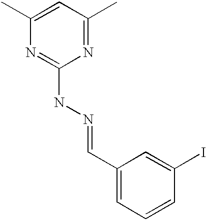

or a pharmaceutically effective derivative, salt, or isomer thereof;

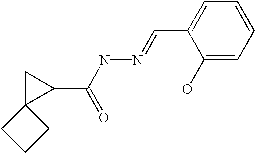

or a pharmaceutically effective derivative, salt, or isomer thereof;

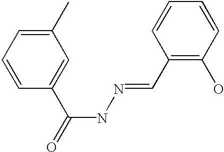

or a pharmaceutically effective derivative, salt, or isomer thereof;

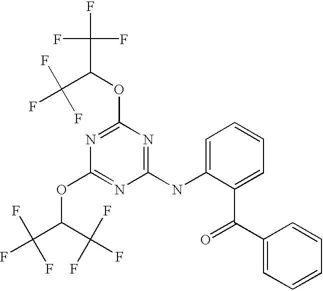

or a pharmaceutically effective derivative, salt, or isomer thereof; or

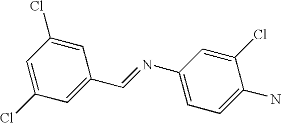

or a pharmaceutically effective derivative, salt, or isomer thereof, and wherein if the compound is haloperidol, phenazopyridine, or R-(−)-deprenyl, then the amyloidogenic protein is not beta-amyloid. In one embodiment, the compounds of the invention are provided in a dose sufficient to decrease or prevent aggregates formed by amyloidogenic proteins, for example, polyglutamine aggregates or inclusions that exist, or might be formed. In another embodiment, cell toxicity is decreased. In the most preferred embodiment, both aggregation and toxicity are decreased. In still another embodiment, the amyloidogenic protein is not beta- amyloid. In yet another embodiment, the cell or animal is contacted with any one of the compounds, or derivatives, salts, or isomers thereof.

In a fourth aspect, the invention features a method for decreasing aggregates or inclusions formed by an amyloidogenic protein repeats in a cell or animal, involving contacting a cell or an animal expressing an amyloidogenic protein with any of bromocriptine mesylate; haloperidol; nabumetone; primidone; hydrocortisone; phenazopyridine; R-(−)-deprenyl hydrochloride; 6a-methylprednisolone 21-hemisuccinate; digoxin; azathioprine; D-cycloserine; red clover; magnesium oxide; N-vanillylnonanmide; neostigmine methyl ether; or a pharmaceutically effective derivative, salt, or isomer thereof; or a compound having the formula selected from any of:

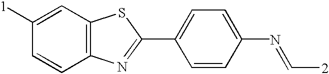

wherein 1 is CH₃ or H, and 2 is

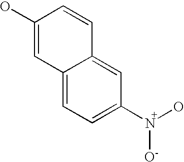

or wherein 1 is CH₃, and 2 is

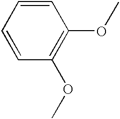

or wherein 1 is CH₃, and 2 is

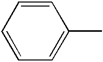

or a pharmaceutically effective derivative, salt, or isomer thereof;

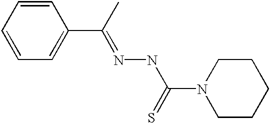

or a pharmaceutically effective derivative, salt, or isomer thereof;

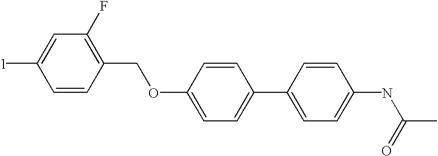

wherein 1 is H or NO₂⁻, or a pharmaceutically effective derivative, salt, or isomer thereof;

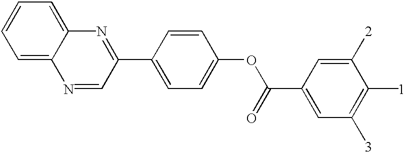

wherein 1 is Cl, and 2 and 3 are H; or wherein 1 and 3 are H; and 2 is NO₂; or wherein 1 is Br, 2 is H, and 3 is NO₂; or wherein 1 is Cl, 2 is H, and 3 is Br, or a pharmaceutically effective derivative, salt, or isomer thereof;

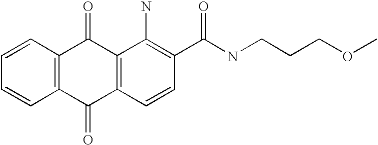

or a pharmaceutically effective derivative, salt, or isomer thereof;

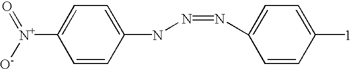

wherein 1 is NO₂, Br, or O₂, or a pharmaceutically effective derivative, salt, or isomer thereof;

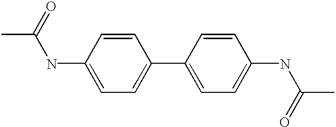

or a pharmaceutically effective derivative, salt, or isomer thereof

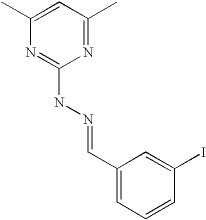

or a pharmaceutically effective derivative, salt, or isomer thereof;

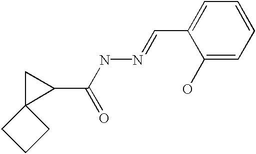

or a pharmaceutically effective derivative, salt, or isomer thereof;

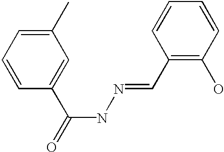

or a pharmaceutically effective derivative, salt, or isomer thereof;

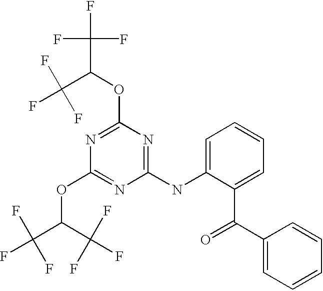

or a pharmaceutically effective derivative, salt, or isomer thereof; or

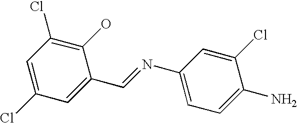

or a pharmaceutically effective derivative, salt, or isomer thereof, and wherein if the compound is haloperidol, phenazopyridine, or R-(−)-deprenyl, then the amyloidogenic protein is not beta-amyloid. In one embodiment, the compounds of the invention are provided in a dose sufficient to decrease or prevent aggregates formed by amyloidogenic proteins, for example, polyglutamine aggregates or inclusions that exist, or might be formed. In another embodiment, cell toxicity is decreased. In a desirable embodiment, both aggregation and toxicity are decreased. In still another embodiment, the amyloidogenic protein is not beta-amyloid. In yet another embodiment, the cell or animal is contacted with any one of the compounds, or derivatives, salts, or isomers thereof.

In another embodiment of any of the above aspects of the invention, the cell is mammalian, preferably human. In yet another embodiment, the animal is a mammal, such as a human or a rodent. In still other embodiments, the cell is a neuron, a muscle cell, a pancreatic cell, or a germ cell, or is ex vivo or in vivo. In a further embodiment, the animal is an animal diagnosed with, or having an increased likelihood of developing a neurodegenerative disease. The neurodegenerative disease may be any of Huntington's disease, spinobulbar muscular atrophy (SBMA), spino-cerebellar ataxia type 1, spino-cerebellar ataxia type 2, spino-cerebellar ataxia type 3, spino-cerebellar ataxia type 6, spino-cerebellar ataxia type 7, dentatorubral-pallidoluysian atrophy, or familial schizophrenia.

In a fifth aspect, the invention features a method for treating a condition, or a symptom associated with a condition, in a subject at risk for having an expressed expanded polyglutamine repeat by administering diphenyldiazo-bis-alpha-napthylaminesulfonate, or a pharmaceutically effective derivative or salt thereof, to the subject.

In a sixth aspect, the invention features a method for treating a condition, or a symptom associated with a condition, in a subject at risk for having an expressed amyloidogenic protein, involving administering any of bromocriptine mesylate; haloperidol; nabumetone; primidone; hydrocortisone; phenazopyridine; R-(−)-deprenyl hydrochloride; 6a-methylprednisolone 21-hemisuccinate; digoxin; azathioprine; D-cycloserine; red clover; magnesium oxide; N-vanillylnonanmide; neostigmine methyl ether; or a derivative, salt, or isomer thereof; or a compound having the formula selected from any of:

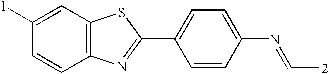

wherein 1 is CH₃ or H, and 2 is

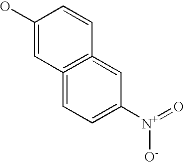

or wherein 1 is CH₃, and 2 is

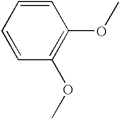

or wherein 1 is CH₃, and 2 is

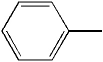

or a pharmaceutically effective derivative, salt, or isomer thereof;

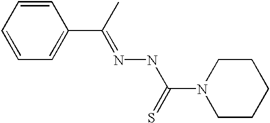

or a pharmaceutically effective derivative, salt, or isomer thereof;

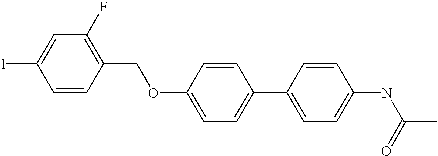

wherein 1 is H or NO₂⁻, or a pharmaceutically effective derivative, salt, or isomer thereof;

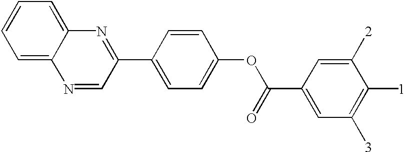

wherein 1 is Cl, and 2 and 3 are H; or wherein 1 and 3 are H, and 2 is NO₂; or wherein 1 is Br, 2 is H, and 3 is NO₂; or wherein 1 is Cl, 2 is H, and 3 is Br, or a pharmaceutically effective derivative, salt, or isomer thereof;

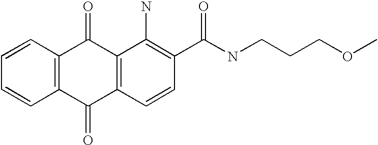

or a pharmaceutically effective derivative, salt, or isomer thereof;

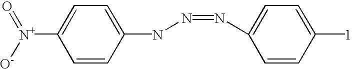

wherein 1 is NO₂, Br, or O₂, or a pharmaceutically effective derivative, salt, or isomer thereof;

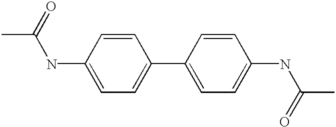

or a pharmaceutically effective derivative, salt, or isomer thereof

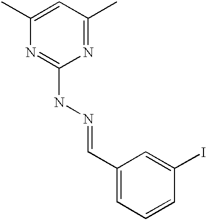

or a pharmaceutically effective derivative, salt, or isomer thereof;

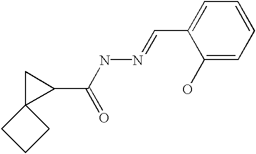

or a pharmaceutically effective derivative, salt, or isomer thereof;

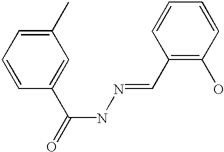

or a pharmaceutically effective derivative, salt, or isomer thereof;

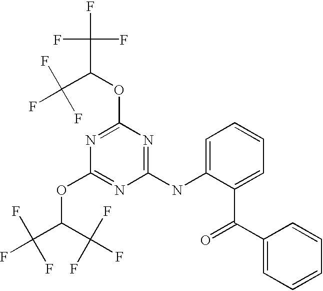

or a pharmaceutically effective derivative, salt, or isomer thereof; or

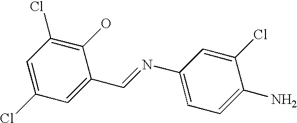

or a pharmaceutically effective derivative, salt, or isomer thereof to the subject.

In one embodiment of the fifth or sixth aspects of the invention, the condition is a neurodegenerative disease. The neurodegenerative disease may be any of Huntington's disease, spinobulbar muscular atrophy (SBMA; also known as Kennedy's disease), spino-cerebellar ataxia type 1, spino-cerebellar ataxia type 2, spino-cerebellar ataxia type 3 (also known as Machado-Joseph disease), spino-cerebellar ataxia type 6, spino-cerebellar ataxia type 7, dentatorubral-pallidoluysian atrophy, or familial schizophrenia. In another embodiment, the condition is male infertility or inclusion-body myositis.

In another embodiment of the fifth aspect of the invention, the condition is caused by expanded polyglutamine repeats. In another embodiment of the sixth aspect of the invention, the condition is caused by an amyloidogenic protein, for example, expanded polyglutamine repeats. In yet another embodiment of the fifth or sixth aspects of the invention, the subject is a mammal, preferably a human.

In still another embodiment of the fifth aspect of the invention, the expressed expanded polyglutamine repeat is one which is resistant to at least one of the following compounds: iota-carrageenan, dextran, minocycline, daunomycin, rolitetracycline, or Chrysamine G, or would be, if allowed to form.

In still another embodiment of the sixth aspect of the invention, the amyloidogenic protein is not beta-amyloid. In yet another preferred embodiment, the subject is contacted with any one of the compounds, or derivatives, salts, or isomers thereof.

In yet another embodiment of the sixth aspect of the invention, if the compound is R-(−)-deprenyl or bromocriptine mesylate, then the condition is not Alzheimer's disease. In another preferred embodiment, if the compound is haloperidol, then the condition is not Alzheimer's disease or Pick's disease. In still another preferred embodiment, if the compound is phenazopyridine, then the condition is not Alzheimer's disease. In further embodiments, if the

In an additional embodiment of any of the first, second, or fifth aspects of the invention, the Congo red derivative is any one of Direct Orange 8, Direct Yellow 26, Direct Yellow 28, Direct Blue 158, Direct Orange 6, Direct Red 1, Direct Orange 1, or Direct Black 51. Structures of these compounds are shown in FIG. 1B-1I.

In an additional embodiment of any of the third, fourth, or sixth aspects of the invention, the derivative is any one of the compounds shown in FIGS. 14A-14F, 15A-15S, or 18A-18O.

By “decreasing cell death” is meant decreasing the number of cells that undergo cell death relative to an untreated control. Preferably cell death is decreased 10%, more preferably 25%, 50%, or 75%, and most preferably 90% relative to a control. A preferred method for measuring cell death is by visually inspecting the cells for morphological and nuclear changes such as cell shrinkage and blebbing and condensed nuclei, as described, for example by Sanchez et al. (Neuron 22:623-633, 1999).

By “cell toxicity” is meant events leading up to the occurrence of cell death. Such events may include, for example, activation of caspase-8. These events may be measured, for example, by viewing the recruitment of caspase-8 by polyglutamine repeats, according to the methods of Sanchez et al. (supra), by determining the cellular ATP level, or by detecting protein synthesis inhibition, as described herein.

By “decreasing cell toxicity” is meant decreasing the number of cells that undergo toxicity relative to an untreated control. Preferably cell toxicity is decreased 10%, more preferably 25%, 50%, or 75%, and most preferably 90% relative to a control. Preferably cell toxicity is measured in cell culture by detection of ATP levels, for example, using the ATPLite™ kit (Packard Co., Meriden, Conn.), or by visual inspection of morphological and nuclear changes, such as cell shrinkage and blebbing and condensed nuclei. In an animal model (e.g., R62 mice carrying a mutation in the human Huntington's disease gene), cell toxicity may be assessed using a rotorod to measure muscle strength.

By an “expanded polyglutamine repeat” is meant translated CAG nucleotide triplet repeats that encode the amino acid glutamine. Preferably the CAG nucleotide repeat is at least 36 glutamines long. Such an expanded polyglutamine repeat is also known as Q36. More preferably, the CAG nucleotide repeat is at least 79 glutamines long, and is also known as Q79.

By “resistant to” is generally meant unaffected by a compound, in that the compound does not affect cell toxicity. As used herein, the term refers to a cell that expresses an expanded polyglutamine repeat and is resistant to the cell viability-protective or aggregate-decreasing effects of a compound administered to the cell. Compounds to which a cell expressing an expanded polyglutamine repeat may be resistant include, for example, iota-carrageenan, dextran, pentosan polysulfate, minocycline, rolitetracycline, and Chrysamine G. Preferably a cell that expresses an expanded polyglutamine repeat and is more resistant to the cell viability-protective or aggregate-decreasing effects of iota-carrageenan, dextran, pentosan polysulfate, minocycline, rolitetracycline, or Chrysamine G than to those same effects mediated by Congo red. More preferably the cell is 20%, 40%, 50%, 75%, or 90% more resistant to the cell viability-protective or aggregate-decreasing effects of iota-carrageenan, dextran, pentosan polysulfate, minocycline, rolitetracycline, or Chrysamine G than to those same effects mediated by Congo red.

By “aggregates” or “inclusions” is meant polypeptides or proteins that have precipitated to form an insoluble complex. As used herein, the aggregates or inclusions consist of polypeptides containing expanded polyglutamine repeats or other amyloidogenic proteins having toxic properties.

By “decreasing or disrupting aggregates or inclusions” is meant decreasing the number or size of aggregates or inclusions formed by polypeptides relative to an untreated control. Preferably the decrease in the number or size of aggregates or inclusions is 10%, more preferably 25%, 50%, or 75%, and most preferably 90% relative to a control. A decrease or disruption of aggregates or inclusions can be detected, for example, by staining a cell or tissue sample with an antibody the binds to the aggregate.

By a “polyglutamine aggregate” is meant polypeptides containing expanded polyglutamine repeats that have precipitated to form an aggregate.

By “treating” is meant submitting or subjecting an animal to a compound which will promote the elimination or reduction of a disease or symptoms of a disease, or which will slow the progression of said disease. For example, an animal may be treated with naturally occurring organic molecules, synthetic organic molecules, peptides, polypeptides, nucleic acid molecules, or components thereof.

By “at risk for having an expressed expanded polyglutamine repeat” is meant that a cell contains a gene that comprises more than 35 CAG nucleotide repeats. Such genes include, but are not limited to, those that encode huntingtin, atrophin-1, ataxin-1, ataxin 3, alpha A1 voltage dependent calcium channel, ataxin-7, and the androgen receptor. In the case that the gene encodes ataxin-2, a CAG nucleotide repeat which is greater than 31 repeats puts a cell containing such a gene at risk for having an expressed expanded polyglutamine repeat.

By a “disease” is meant a condition of a living animal that impairs the normal performance or function of the animal.

By a “condition” is meant a state of being or feeling. Conditions include, but are not limited to, neurodegenerative diseases and the symptoms associated with neurodegenerative diseases, inclusion-body myositis, or infertility.

By a “subject at risk for a disease” is meant a subject identified or diagnosed as having a disease or having a genetic predisposition or risk for acquiring a disease using the methods of the invention and techniques available to those skilled in the art.

By a “neurodegenerative disease” is meant a disease characterized by neuronal cell death. Examples of neurodegenerative diseases include, but are not limited to, Alzheimer's disease, Huntington's disease, stroke, amyotropic lateral sclerosis, multiple sclerosis, Lewy body disease, Menkes, disease, Wilson disease, Creutzfeldt-Jakob disease, Fahr disease, Parkinson's disease, spino-cerebellar ataxia type 1, spino-cerebellar ataxia type 2, spino-cerebellar ataxia type 3 (also know as Machado-Joseph disease), spino-cerebellar ataxia type 6, spinal bulbar muscular disease (also known as Kennedy's disease), dentatorubral-pallidoluysian atrophy, prion disease, familial amyloidotic polyneuropathy, multiple system atrophy, supranuclear palsy, Pick's disease, and familial schizophrenia.

By a “neuronal cell” is meant a cell of ectodermal embryonic origin derived from any part of the nervous system of an animal, such as a human or a rodent. Neurons express well-characterized neuron-specific markers that include neurofilament proteins, MAP2, and class III β-tubulin. Included as neurons are, for example, hippocampal, cortical, midbrain dopaminergic, motor, sensory, sympathetic, septal cholinergic, and cerebellar neurons.

By a “germ-line cell” is meant a cell, progenitor, or progeny thereof, which is a product of a meiotic cell division. Preferably, the germ-line cell of the invention is a male germ-line cell and resides in the testis.

By a “pharmaceutically effective derivative” is meant a structural derivative having a chemical modification of the compound which does not modify the ultimate level of cell death or toxicity, but which does enhance bioavailability, solubility, or stability in vivo or ex vivo, or which reduces the toxicity or dosage required. Such modifications are known to those skilled in the field of medicinal chemistry.

By an “isomer” is meant one of two or more molecules that have the same chemical formula but have a different stereochemical arrangement of the atoms. Preferably an isomer of any of the compounds of the present invention is a stereoisomer that has the same connectivity, but differs in the arrangement of its atoms in space, compared to a compound of the present invention.

By an “amyloidogenic protein” is meant a protein or polypeptide containing anti-parallel beta sheets, forming a structure or fibril similar to that of an amyloid polypeptide. Example of amyloidogenic proteins include, but are not limited to, serum amyloid A protein, islet amyloid polypeptide, isolated atrial amyloid, expanded polyglutamine repeat polypeptides, calcitonin, scrapie protein, beta 2 microglobulin, beta 2 precursor protein, cystatin C, gelsolin, apolipoproteins AI and SAA, transthyretin, IgG 1, immunoglobulin light chain kappa, and immunoglobulin light chain lambda. Additional amyloidogenic proteins include expanded polyglutamine repeat polypeptides, mutated tau, alpha synuclein, and superoxide dismutase-1 polypeptides.

The present invention provides a number of advantages. For example, the methods described herein allow for a decrease in cell death. The invention also provides compounds and methods for treating diseases in which cell death occurs. These compounds and methods can be used to treat conditions such as a neurodegenerative disease or infertility, and conditions associated with such diseases, and are especially useful for treating conditions in which expanded polyglutamine repeats or other amyloidogenic proteins are expressed in the cells associated with the condition, for example, for treating the neurons in a patient with a neurodegenerative disease.

Other features and advantages of the invention will be apparent from the following detailed description and from the claims.

### BRIEF DESCRIPTION OF THE DRAWINGS

FIG. 1A is a schematic representation of the structure of Congo red.

FIGS. 1B-1I are schematic representations of structural derivatives of Congo red.

FIG. 2A is a graph of the effect of various concentrations of Congo red on expanded polyglutamine-induced cell death. Neuroblastoma (SHSY) cells were transfected with a Q79-GFP plasmid. The shaded bars represent the percentage of cells expressing Q79-GFP. The striped bars represent the percentage of cell death in cells expressing Q79 and subsequently treated with Congo red (1.4 nM or 14 nM), or left untreated (control).

FIGS. 2B-2D are a series of scanned images of neuroblastoma (SHSY) cells expressing Q79-GFP and subsequently treated with Congo red (1.4 nM or 14 nM), or left untreated (control), as detected by fluorescence microscopy.

FIGS. 3A-3F are a series of photographs showing the effect of a variety of compounds on the decrease of pre-formed expanded polyglutamine oligomers. Q79-GFP was expressed and formed into aggregates. These aggregates were left untreated (NT) or contacted with a variety of compounds (minocycline, daunomycin, rolitetracycline, Congo red, or Chrysamine G) and evaluated for the effect of each compound on the decrease of the aggregates.

FIG. 4A is two graphs showing the effect of Congo red, tetracyclines, and sulfated polyanions on polyglutamine-induced cell death in, each graph representing a separate experiment. HeLa cells transfected with a Q79-GFP construct were treated with 100 μM of minocycline (mino), Chrysamine G (CG), Rolitetracycline (Ro), iota carrageenan (iota), dextran 500 (dextran), or Congo red (CR), six hours after transfection. The amount of cell death was determined 48 hours after transfection by morphological criteria as previously described (Sanchez et al., supra).

FIG. 4B is a graph showing the effect of Congo red (CR) on the induction of cell death by receptor-mediated pathways. HeLa cells transfected with a Q79-GFP plasmid were treated with TNF-a or anti-FasR and cycloheximide (CHX), in the presence or absence of CR (100 μM). The amount of cell death was measured 72 hours after transfection.

FIG. 5A is a graph showing the viability of HeLa cells expressing or not expressing expanded polyglutamine repeats (Q79-GFP). Levels of ATP in 4×10⁵ cells transiently transfected with Q79-GFP, assayed 48 hours after transfection, are expressed in arbitrary luminescence units.

FIG. 5B is a graph showing the viability of HeLa cells expressing or not expressing expanded polyglutamine repeats (Q79-GFP), in the presence or absence of the caspase inhibitor ZVAD. Levels of ATP in each well containing 1500 cells transiently transfected with Q79-GFP, and treated or not treated with ZVAD, assayed 48 hours after transfection, are expressed in arbitrary luminescence units.

FIG. 5C is a graph showing the effect of Congo red (100 μM) on polyglutamine-induced cell toxicity. Levels of ATP in 4×10⁵ cells transiently transfected with Q79, and treated with varying concentrations of Congo red, assayed 72 hours after transfection, are expressed in arbitrary luminescence units.

FIG. 5D is a graph showing the effect of Congo red (100 μM) on polyglutamine-induced cell toxicity, as assessed by measuring the ratio of ATP levels in Conge red-treated cells, compared to vehicle-treated cells, and on protein synthesis inhibition, as assessed by measuring luciferase activity in Congo red-treated cells, compared to vehicle-treated cells.

FIG. 5E is a scanned image of an autoradiogram showing the effect of Congo red on caspase-8 activation and endogenous heat shock protein levels (HSP 40 and HSP 70) in expanded polyglutamine repeat (Q79) expressing cells, as well as the ATP levels and percent of cell death occurring in each sample.

FIG. 5F is a graph of the effects of Congo red, ZVAD, or vehicle (NT) on cell death when the cells are induced to die by receptor mediated pathways (TNF-α/cycloheximide (CHX), Fas/cycloheximide (CHX)), or necrosis (hydrogen peroxide).

FIG. 5G is a graph of the effects of Congo red on cell death induced by adapter protein oligomerization (Daxx, RIP, FADD).

FIG. 5H is a graph of the effects of Congo red on cell death induced by caspase over-expression(caspases-8, -1, or -11).

FIG. 5I is a set of scanned images of HeLa cells expressing Q79-GFP and subsequently treated with Congo red (100 μM), or ZVAD (100 μM), as detected by fluorescence microscopy.

FIG. 6A is a graph of the percent of binding of Congo red to expanded polyglutamine repeats Q81, Q62, and Q19.

FIG. 6B is a graph of the percent of binding of various compounds to amyloid-like Q81 aggregates measured using a chemical absorption assay. The anti-amyloid compounds minocycline, Chrysamine G, Rolitetracycline, iota carrageenan, dextran 500 (dextran), or Congo red (25 μM of each) were pre-absorbed with Q81 GST recombinant protein and the percentage absorbance of the compound remaining in the supernatant after absorption with GST-Q81 beads is plotted on the y-axis.

FIG. 6C is a set of scanned images of the disruption of pre-formed Q79-GFP oligomers by Congo red, minocycline, or rolitetracycline. Lysates from Q79-GFP expressing cells were treated with 25 μM of the indicated compound, and visualized by fluorescence microscopy.

FIG. 6D is a scanned image of a filter showing the effect of Congo red on pre-formed oligomers as assessed by the filter assay described herein. Cells were treated with PBS or 100 μM of Congo red 6 hours after transfection with Q79-GFP. Lysates from these cells were obtained 42 hours later, and passed through a 0.2 micron pore filter. In addition, Q79 aggregates were treated with Congo red after they were recovered from cells to test for disruption of pre-formed aggregates.

FIG. 6E is a graph showing the effect of Congo red on Q79-Q79 interaction, as assayed by FRET analysis.

FIG. 7A is a graph of the effect of Congo red or ZVAD on the recruitment of caspase-8, a death domain, or proteins containing short polyglutamine repeats by expanded polyglutamine oligomerization in cells transfected with a Q79-GFP expression construct and a caspase-8 (GFP-casp 8DN), death domain (GFP-FADD DN), or short polyglutamine (HD-1 (Q25)-GFP) protein recruitment.

FIG. 7B is a set of scanned images of the cells of FIG. 7A, containing GFP positive aggregates, detected by fluorescence microscopy.

FIG. 7C is an image of an immunoblot of lysates from Q79 expressing cells that were treated with PBS or Congo red during or after transfection, as indicated in the Figure and passed through a 0.2 micron filter. Endogenous caspase-8 and the adapter protein FADD were trapped in the filter in cell lysates containing oligomerized polyglutamine repeats, and probed using a caspase-8 or FADD antibody.

FIG. 8A is a set of scanned autoradiograms showing the effect of Congo red on expanded polyglutamine repeat turn-over, as assessed by relative levels of S³⁵ labeled polyQ (Q79) protein, with or without Congo red treatment. Q79-HA expressing cells were metabolically labeled with S³⁵ methionine for one hour. Cells were harvested one and 24 hours after labeling. PolyQ protein was immunoprecipitated with an anti-HA antibody, and the proteins were separated by electrophoresis before blotting.

FIG. 8B is a graph showing the effect of Congo red on the rate of HA-Q79 degradation, quantified from the results of FIG. 8A.

FIG. 8C is a graph of the effect of effect of Congo red, ZVAD, or the proteosome inhibitor MG132 on total cellular protein degradation in non-transfected HeLa cells.

FIG. 9 is a graph showing the effect of intraperitoneal injection of Congo red on the motor performance in the mouse model of Huntington's disease (R62). Wild type mice or R62 transgenic mice carrying a polyglutamine repeat (R62Tg) were injected with either 0.5 ml of 1 mg/ml Congo red in phosphate buffered saline (PBS) or PBS alone and tested for their muscle strength by measuring their ability to remain on the rotorod at 10 rpm for a maximum period of 60 seconds.

FIG. 10A is a graph showing the effect of Congo red or PBS (vehicle) on body weight in HD transgenic mice between 9 and 11 weeks of age (top graph) and between 9 and 13 weeks of age (bottom graph). Mice were infused intraperitoneally (IP) or by intracerebroventricular cannula (ICV) implanted into the predetermined coordinates on the left ventricle, or by both routes.

FIG. 10B is a table showing the effect of intraperitoneal delivery of Congo red or PBS (vehicle) on fasting glucose levels in wild-type (WT) or HD transgenic mice.

FIG. 10C a set scanned images of general aspects of Congo red-treated and PBS (vehicle)-treated HD transgenic mice (R62) at 12.5 wks.

FIG. 10D is a set of scanned images of the effect of Congo red or PBS (vehicle) on motor coordination, as assessed by the “ink” test in HD transgenic mice three weeks after Congo red or vehicle treatment. The “ink” test was used to determine the changes in stride length and the overlap of steps characteristic of movement disorders using red and green food coloring to mark the front and back paws, respectively. Changes in stride length of individual mice from one set of paws (brackets) and the differences in the step overlapping patterns (open arrows) three weeks after vehicle or Congo red treatment were assayed.

FIG. 10E is a table of the quantification of changes in stride length in HD transgenic mice treated with either Congo red or PBS (vehicle) (#, n=5 in each cohort), assessed as described in FIG. 10D.

FIG. 10F is a graph of the effect of PBS (vehicle; delivered intraperitoneally (IP)) or Congo red, infused intraperitoneally (IP), by intracerebroventricular cannula (ICV) implanted into the predetermined coordinates on the left ventricle, or by both routes, on motor performance, as assessed by latency to fall measurements using a rotorod.

FIG. 10G is a graph of the effect of PBS (vehicle; delivered intraperitoneally) or Congo red, infused intraperitoneally, on survival of HD transgenic mice.

FIG. 11A is a light micrograph of the immunolocalization of expanded polyglutamine repeats in the basal ganglia of an R62 mouse model of Huntington's disease prior to Congo red infusion (which began at postnatal week 9).

FIG. 11B is a light micrograph of the immunolocalization of expanded polyglutamine repeats in the basal ganglia of an R62 mouse model of Huntington's disease after intracerebroventricular cannula (ICV) delivery of PBS (vehicle) at postnatal week 12.5).

FIG. 11C is a light micrograph of the immunolocalization of expanded polyglutamine repeats in the hippocampus and basal ganglia of an R62 mouse model of Huntington's disease after intraperitoneal IP delivery of PBS (vehicle) at postnatal week 12.5.

FIG. 11D is a light micrograph of the immunolocalization of expanded polyglutamine repeats in the basal ganglia of an R62 mouse model of Huntington's disease after intracerebroventricular cannula (ICV) delivery of Congo red at postnatal week 12.5.

FIG. 11E is a light micrograph of the immunolocalization of expanded polyglutamine repeats in the hippocampus of an R62 mouse model of Huntington's disease after intraperitoneal (IP) delivery of Congo red at postnatal week 12.5.

FIG. 11F is a scanned image of the immunolocalization of expanded polyglutamine repeats in the basal ganglia of an R62 mouse model of Huntington's disease after intraperitoneal (IP) delivery of Congo red at postnatal week 12.5.

FIG. 12A is a graph of the effect of Congo red and derivatives of Congo red on Q79-induced cytotoxicity, as assessed by measuring ATP levels. Results are shown as a percent of ATP in Q79-GFP-expressing cells compared to GFP-expressing cells.

FIG. 12B is a graph of the effect of Congo red and derivatives of Congo red on Q79-induced cytotoxicity, as assessed by luciferase activity. Results are shown as a percent of luciferase activity in cells treated with the compound compared to cell treated with vehicle only. NT=no treatment; EB=Evans blue; ThioS=thioflavin S; and ThioT=thioflavin T.

FIG. 13A is a graph of the effect of small molecules from a ChemBridge Library and ZVAD on Q79-induced cytotoxicity, as assessed by luciferase activity. Results are shown as a percent of luciferase activity in cells treated with the compound compared to cells treated with vehicle only.

FIG. 13B is a graph of the effect of small molecules from a ChemBridge Library and ZVAD on Q79-induced cytotoxicity, as assessed by measuring ATP levels. Results are shown as a percent of ATP in cells treated with the compound compared to cells treated with vehicle only.

FIG. 14A shows the structure of PQIA, a generic structure with proposed groups 1 and 2 for PQIA derivatives PQIA-1, PQIA-2, and PQIA-3 from the ChemBridge Library, along with their ChemBridge product numbers. Also shown are graphs of the effects of PQIA and its derivatives on Q79-induced cytotoxicity, as assessed by luciferase activity, and ATP levels. Results are shown as a percent of luciferase activity or ATP in cells treated with the compound, compared to cells treated with vehicle only.

FIG. 14B shows the structure of PQIB, a generic structure and proposed group 1 for PQIB derivatives PQIB-1, and PQIB-2 from the ChemBridge Library, along with their ChemBridge product numbers. Also shown are graphs of the effects of PQIB and its derivatives on Q79-induced cytotoxicity, as assessed by luciferase activity, and ATP levels. Results are shown as a percent of luciferase activity or ATP in cells treated with the compound, compared to cells treated with vehicle only.

FIG. 14C shows the structure of PQIC, a generic structure and proposed group 1 for PQIC derivative PQIC-1 from the ChemBridge Library, along with their ChemBridge product numbers. Also shown are graphs of the effects of PQIC and its derivative on Q79-induced cytotoxicity, as assessed by luciferase activity, and ATP levels. Results are shown as a percent of luciferase activity or ATP in cells treated with the compound, compared to cells treated with vehicle only.

FIG. 14D shows the structures of PQID and PQIM, a generic structure and proposed groups 1, 2, and 3 for PQID derivatives PQID-1 and PQID-2 from the ChemBridge Library, along with their ChemBridge product numbers. Also shown are graphs of the effects of PQID, PQIM, and its derivative on Q79-induced cytotoxicity, as assessed by luciferase activity, and ATP levels. Results are shown as a percent of luciferase activity or ATP in cells treated with the compound, compared to cells treated with vehicle only.

FIG. 14E shows the structure of PQIF, a generic structure and proposed group 1 for PQIF derivatives PQIF-1 and PQIF-2 from the ChemBridge Library, along with their ChemBridge product numbers. Also shown are graphs of the effects of PQIF and its derivative on Q79-induced cytotoxicity, as assessed by luciferase activity, and ATP levels. Results are shown as a percent of luciferase activity or ATP in cells treated with the compound, compared to cells treated with vehicle only.

FIG. 14F shows the structure of PQIG, a generic structure and proposed groups 1, 2, 3, 4, 5, and 6 for PQIG derivatives PQIG-1, PQIG-2, PQIG-3, PQIG-4, PQIG-5, and PQIG-6 from the ChemBridge Library, along with their ChemBridge product numbers. Also shown are graphs of the effects of PQIF and its derivative on Q79-induced cytotoxicity, as assessed by luciferase activity, and ATP levels. Results are shown as a percent of luciferase activity or ATP in cells treated with the compound, compared to cells treated with vehicle only.

FIGS. 14G-14L show the structures of PQIE, PQIK, PQIM, PQII, PQIL, and PQIN-1, respectively, along with their ChemBridge product numbers.

FIGS. 15A-15S show the structures of additional derivatives of PQID (FIGS. 15A-15J), PQIA (FIG. 15K), and PQIB (FIGS. 15L-15S).

FIGS. 16A-16M show the structures of additional derivatives of Congo red.

FIGS. 17A-17O are graphs of the effect of various concentrations of FDA-approved drugs (indicated in each graph) on Q79-induced HeLa cell cytotoxicity, as assessed by luciferase activity. Results in each graph are shown as a percent of luciferase activity in cells treated with the compound compared to cells treated with vehicle only.

FIGS. 18A-18O show the structures of FDA-approved drugs and derivatives that can be used to decrease cell death or toxicity in animals or cell expressing amyloidogenic proteins.

FIG. 19 is a graph of the percent binding of the indicated FDA-approved drugs to GST-Q81 beads, expressed as the percent of compound that did not bind to the Q81 beads, as measured by absorbance of the compounds at their optimal wavelength.

### DETAILED DESCRIPTION OF THE INVENTION

Described herein are methods for decreasing cell toxicity or death, as well as for treating a condition in a subject. Techniques for carrying out the methods of the invention are now described in detail.

Congo Red Disrupts Aggregates that Other Compounds can not Disrupt

The methods of the present invention involve expanded polyglutamine repeats that are resistant to at least one of the following compounds: iota-carrageenan, dextran, minocycline, rolitetracycline, or Chrysamine G. These compounds are capable of disrupting amyloidogenic proteins, however, they are not capable of disrupting or decreasing aggregates formed by expanded polyglutamine repeats, as detailed in Example 4. Therefore, the specific use of Congo red in the present invention is unique, as the use of Congo red to treat diseases that are resistant to the above compounds provides a new avenue for the treatment of conditions, such as neurodegenerative diseases.

Protection of Expanded Polyglutamine-Induced Cell Death by Congo Red

Cells that express expanded polyglutamine repeats undergo cell death. It appears that this cell death occurs as a result of a toxic gain-of-function that is deleterious to the neurons affected in diseases such as Huntington's disease, and spino-cerebellar ataxias. Congo red, or a structural derivative or salt thereof, may be used to prevent cell death or toxicity induced by such expanded polyglutamine repeats. The expanded polyglutamine repeats are resistant to at least one of the compounds chosen from the group consisting of iota-carrageenan, dextran, pentosan polysulfate, minocycline, rolitetracycline, and Chrysamine G. The cells may be mammalian, such as human or rodent cells.

Decrease of Aggregates or Inclusions Formed by Expanded Polyglutamine Repeats by Congo Red

Congo red may also be used to decrease aggregates or inclusions formed by expanded polyglutamine repeats. These aggregates may be formed in vivo or ex vivo. Congo red is then applied to the pre-formed aggregates and the aggregates decrease in size ad number. The expanded polyglutamine repeats of the present invention may be resistant to at least one of the compounds chosen from the group consisting of iota-carrageenan, dextran, minocycline, rolitetracycline, and Chrysamine G. The ability of Congo red to decrease preformed polyglutamine aggregates is important, as such a decrease results in elimination of the toxic gain-of-function that occurs in a cell expressing expanded polyglutamine repeats. This decrease, in turn, results in increased cell viability.

The Use of Congo Red to Prevent Cell Toxicity and Death

Congo red may be used prophylactically to prevent the occurrence of cell death and toxicity in patients who are diagnosed as having a disease characterized by expanded polyglutamine repeats, or to be at risk for developing such a disease. For example, a patient diagnosed as having more than 35 CAG nucleotide repeats in a gene that causes Huntington's disease, dentatorubral-pallidoluysian atrophy, or spino-cerebellar ataxia type 1, 3, 6, or 7, or more than 31 CAG nucleotide repeats in a gene that causes spino-cerebellar ataxia type 2 may be administered Congo red to prevent cell toxicity or death before the patient is symptomatic. Congo red may be administered by any standard dosage and route of administration, as described below.

In Vitro and In Vivo Models

The ability of Congo red to decrease polyglutamine aggregates, or prevent cell toxicity or death was first tested in a cell culture model. Suitable cell culture models include neuroblastoma cells, HeLa cells, primary neuronal cells, and primary embryonic neuronal cells. Once Congo red was shown to effectively decrease cell toxicity or death, or to decrease polyglutamine aggregates in an in vitro system, by the methods described above, it was tested further in animal models. Particularly useful animal models include mouse, rat, and C. elegansn models of cell death or neurodegenerative diseases, for example, the murine R62 line, a model for Huntington's disease as described by Carter et al. (J. Neurosci. 19:3248-3257, 1999) or the C. elegans model of Huntington's disease as described by Faber et al. (Proc. Natl. Acad. Sci. USA 96:179-184, 1999). Upon demonstration that Congo red effectively decreases cell toxicity or death caused by a particular polyglutamine repeat, Congo red may be used as a therapeutic to decrease or prevent cell death or toxicity or to decrease polyglutamine aggregates, as appropriate.

Protection of Amyloidogenic Protein-Induced Cell Death

Cells that express insoluble protein aggregates, for example, amyloidogenic protein aggregates undergo cell death. It appears that this cell death occurs as a result of a toxic gain-of-function that is deleterious to the cells, for example, neurons affected in diseases, such as Huntington's disease, familial amyotrophic lateral sclerosis, inclusion-body myositis, and spino-cerebellar ataxias. The compounds described herein, or structural derivatives, salts, or isomers thereof, may be used to prevent cell death or toxicity induced by amyloidogenic proteins. The cells may be mammalian, such as human or rodent cells.

Cells that express amyloidogenic proteins, for example, expanded polyglutamine repeats, or that are generated to produce amyloidogenic proteins, for example, by transfection of a nucleic acid molecule encoding an amyloidogenic polypeptide into a cell, using standard molecular biology techniques (e.g., Ausubel et al., Current Protocols in Molecular Biology, John Wiley & Sons, New York, N.Y., 1998, hereby incorporated by reference) are tested to determine the effect of candidate modulators of cell toxicity and cell death on the cell, using, for example, methods described herein. These candidate compounds can also be assessed for their ability to decrease aggregates or inclusions formed by an amyloidogenic protein by expressing an amyloidogenic polypeptide in a cell and assaying for the effect of the compound on the number and size of the aggregates or inclusions. Compounds that effectively decrease cell toxicity or death, or decrease aggregates or inclusions formed by an amyloidogenic protein in an in vitro system, are then tested in animal models. Particularly useful animal models include mouse, rat, and C. elegans models of cell death or neurodegenerative diseases, for example, the murine R62 model for Huntington's disease described above, or the C. elegans model of Huntington's disease as described by Faber et al. (Proc. Natl. Acad. Sci. USA 96:179-184, 1999). Upon demonstration that the candidate compound effectively decreases cell toxicity or death caused by an amyloidogenic protein, the compound may be used as a therapeutic to decrease or prevent cell death or toxicity or to decrease aggregates or inclusions formed by amyloidogenic proteins, as appropriate.

Therapy

Congo red may be administered within a pharmaceutically acceptable diluent, carrier, or excipient, in unit dosage form. Conventional pharmaceutical practice may be employed to provide suitable formulations or compositions to administer Congo red, or a derivative, salt or isomer or Congo red to patients suffering from, or at risk of suffering from, a disease that is characterized by polyglutamine repeats, aggregates, or inclusions.

The additional compounds described herein, and their derivatives, salts, and isomers, may administered within a pharmaceutically acceptable diluent, carrier, or excipient, in unit dosage form. Conventional pharmaceutical practice may be employed to provide suitable formulations or compositions to administer these compounds, or a derivatives, salts, or isomers to patients suffering from, or at risk of suffering from, a disease that is characterized by an amyloidogenic protein, aggregates, or inclusions.

Administration of the compounds described herein may begin before the patient is symptomatic. Any appropriate route of administration may be employed, for example, administration may be parenteral, intravenous, intraarterial, subcutaneous, intramuscular, intracranial, intraorbital, ophthalmic, intraventricular, intracapsular, intraspinal, intracisternal, intraperitoneal, intranasal, aerosol, by suppositories, or oral administration. Therapeutic formulations of Congo red may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington's Pharmaceutical Sciences” (Remington: The Science and Practice of Pharmacy” (19th ed., ed. A. R. Gennaro A R., 1995, Mack Publishing Company, Easton, Pa.). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for Congo red include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

If desired, treatment with a compound identified according to the methods described above, may be combined with more therapies for diseases characterized by cell death, or their secondary symptoms. For example, the compounds described herein may be combined with therapeutics used to treat depression (e.g., tricyclic antidepressants), manic behavior (e.g., lithium or valproate), or choria (e.g., monamine depleting drugs, such as reserpine). In addition, two or more of the compounds described herein may be combined for therapeutic use.

Structural Derivatives of Congo Red for Use in Decreasing Cell Toxicity or Death or Decreasing Polyglutamine Aggregates

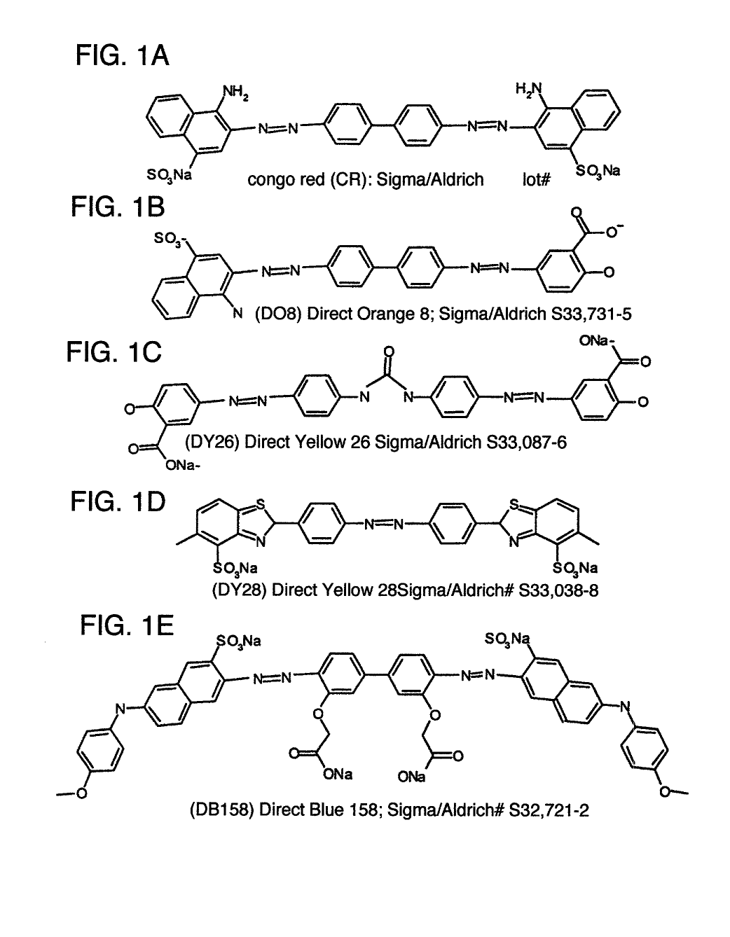

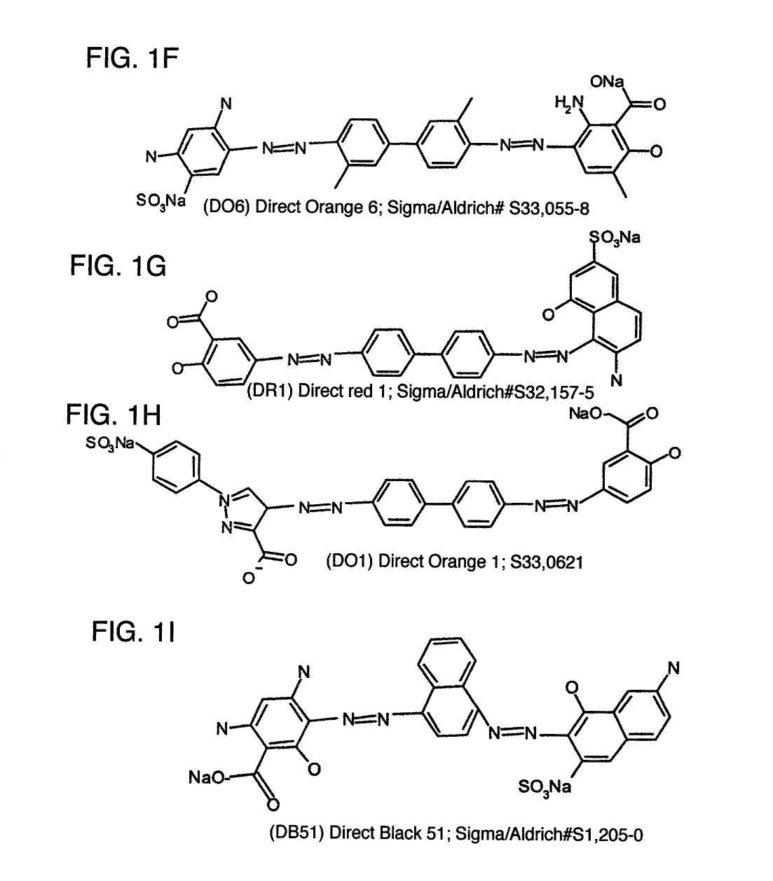

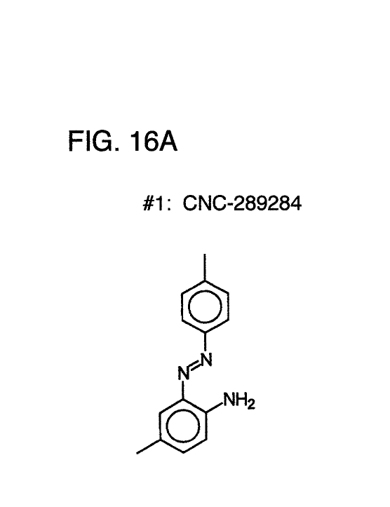

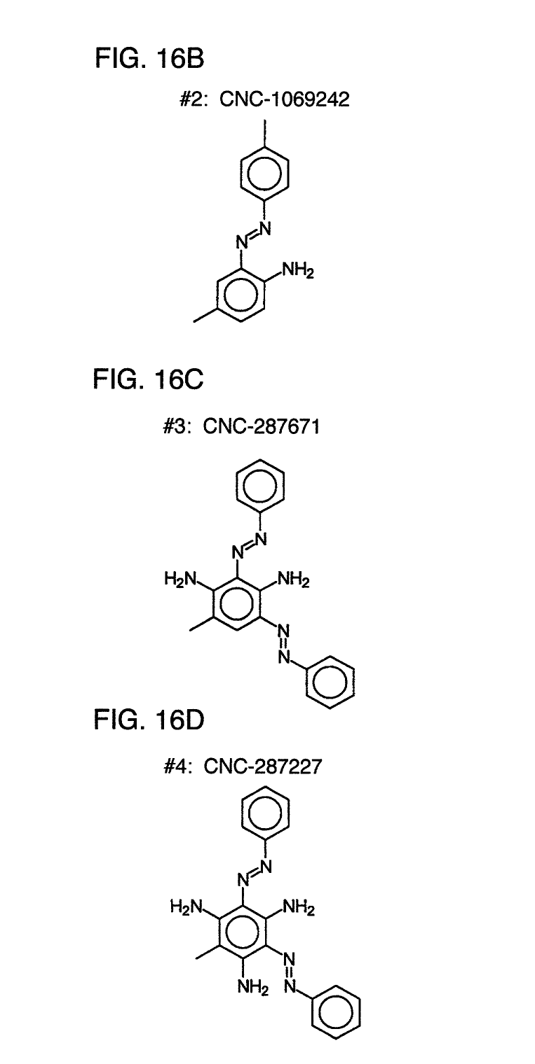

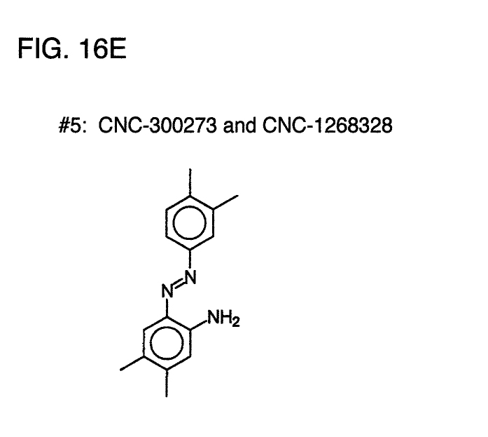

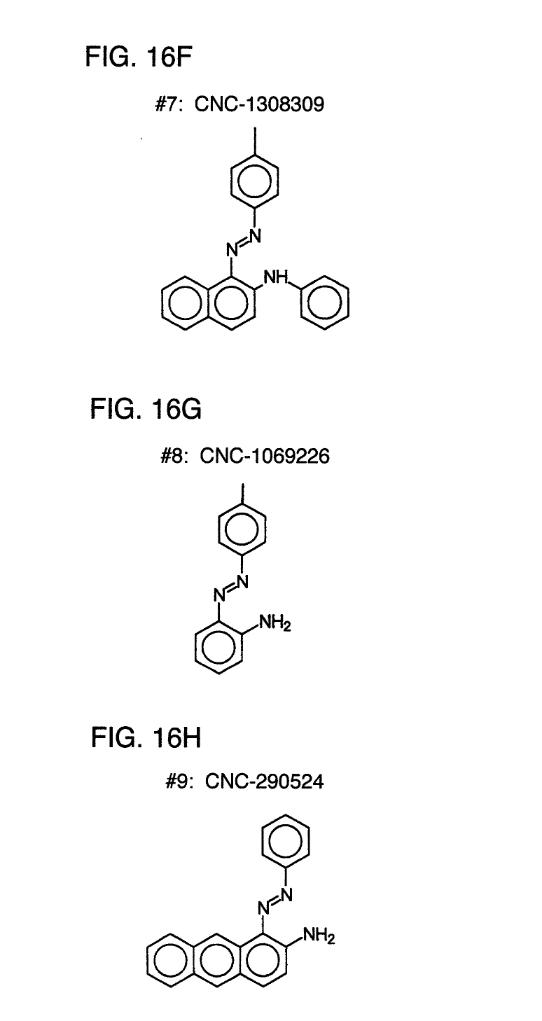

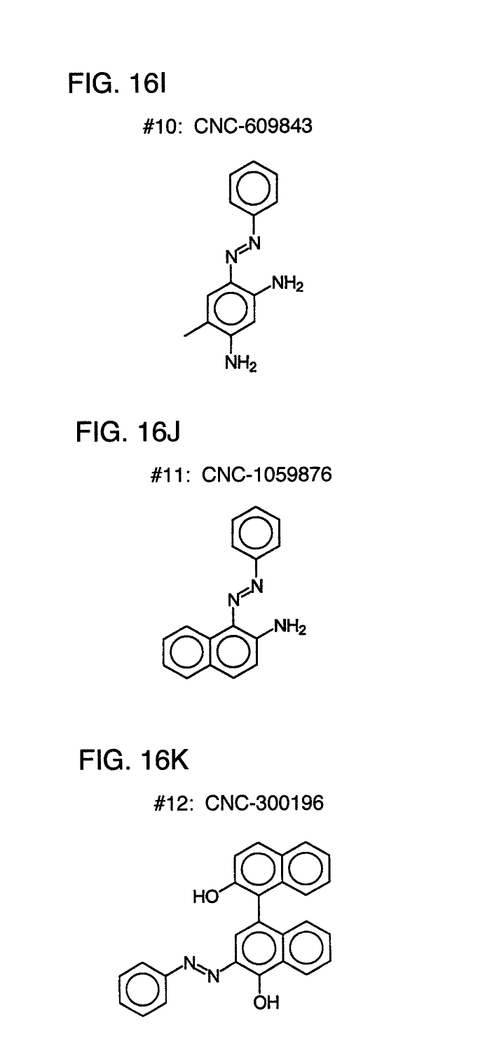

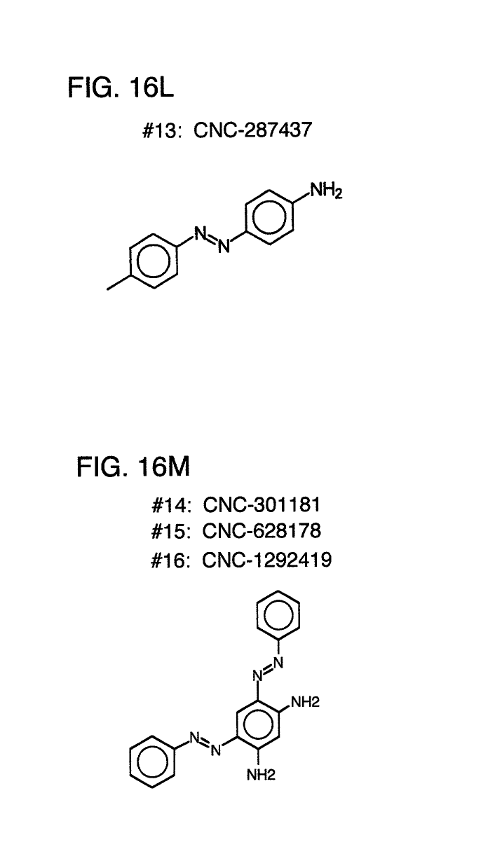

Congo red may be structurally modified and subsequently used to decrease cell toxicity or death, or to decrease polyglutamine aggregates. These derivatives may also be used to treat a condition characterized by the presence of polyglutamine aggregates, or to prophylactically treat a subject at risk for developing a condition characterized by polyglutamine aggregates. For example, Congo red may be modified to form derivatives, using techniques known in the art. The structure of Congo red is provided in FIG. 1A, and Congo red structural derivatives Direct Orange 8 (DO8), Direct Yellow 28 (DY26), Direct Yellow (DY28), Direct Blue (DB158), Direct Orange 6 (DO6), Direct Red 1 (DR1), Direct Orange 1 (DO1), and Direct Black 1 (DB1), which may be used to carry out the invention are provided in FIGS. 1B-I. Additional Congo red derivatives are provided in FIGS. 16A-16M.

Structural Derivatives of Compounds for Use in Decreasing Cell Toxicity or Death or Decreasing Aggregates Formed by Amyloidogenic Proteins

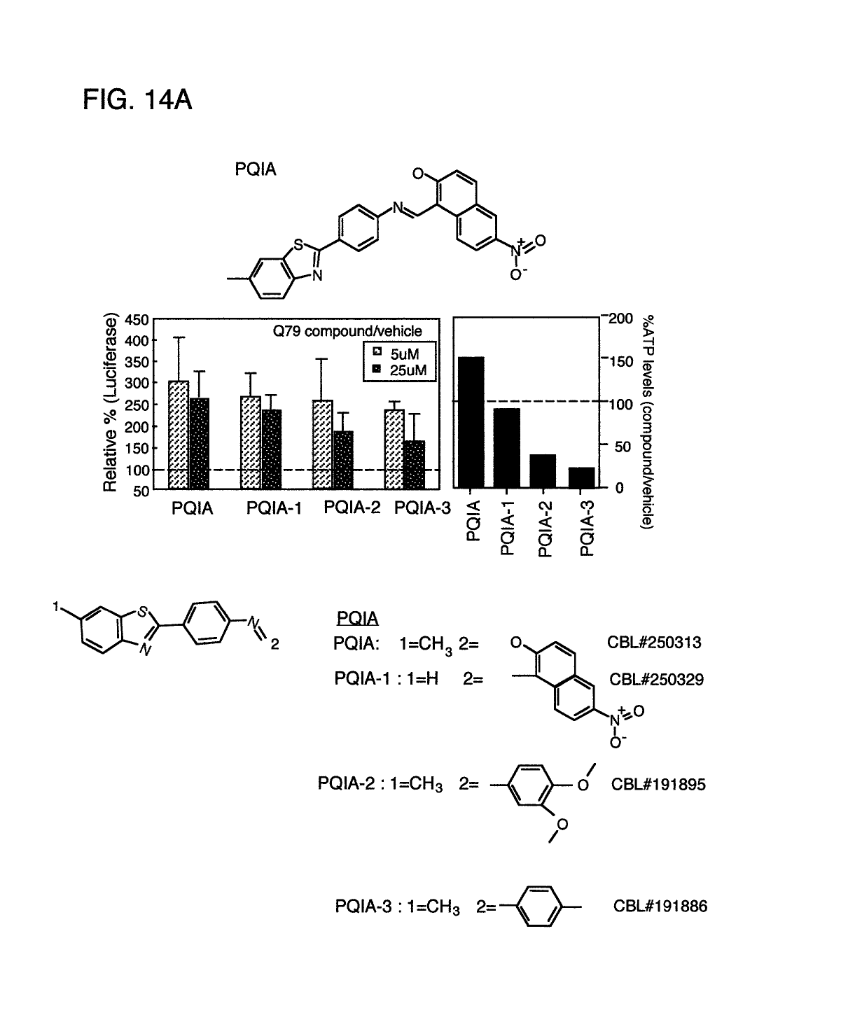

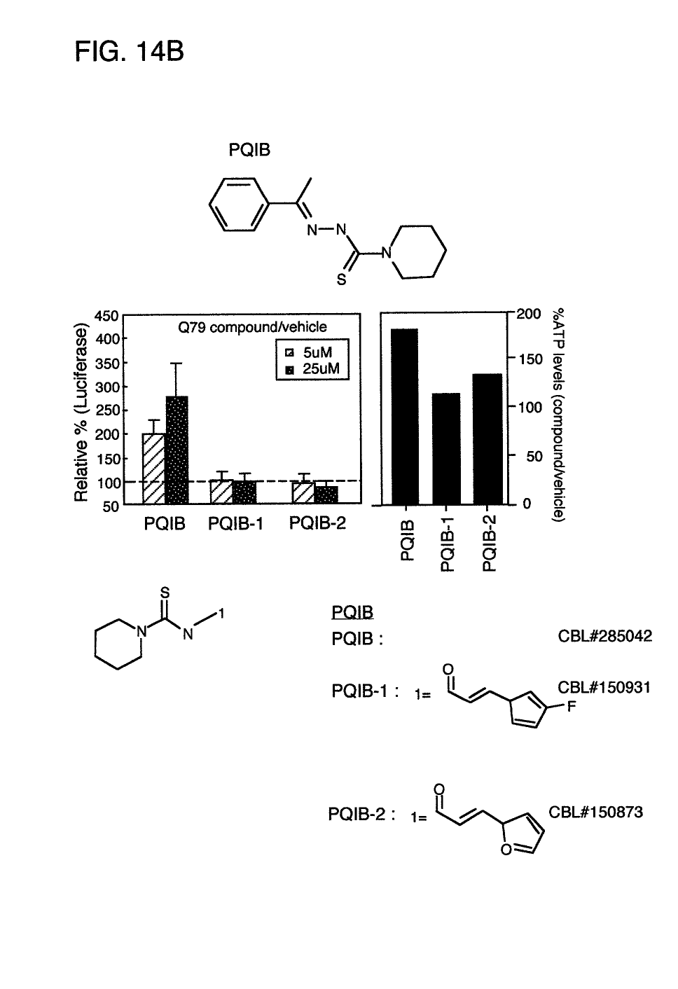

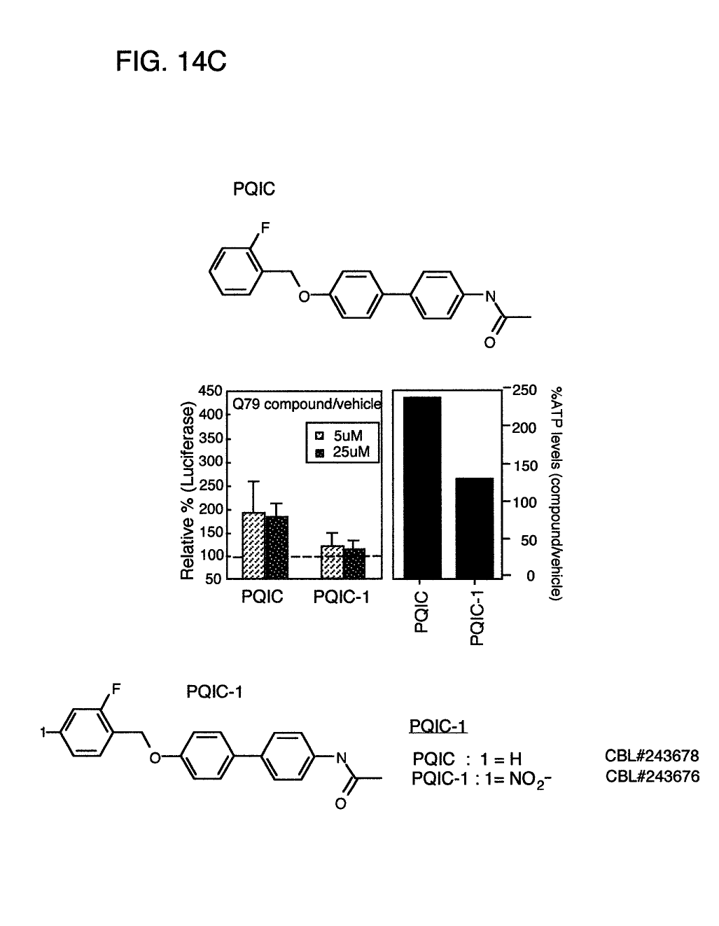

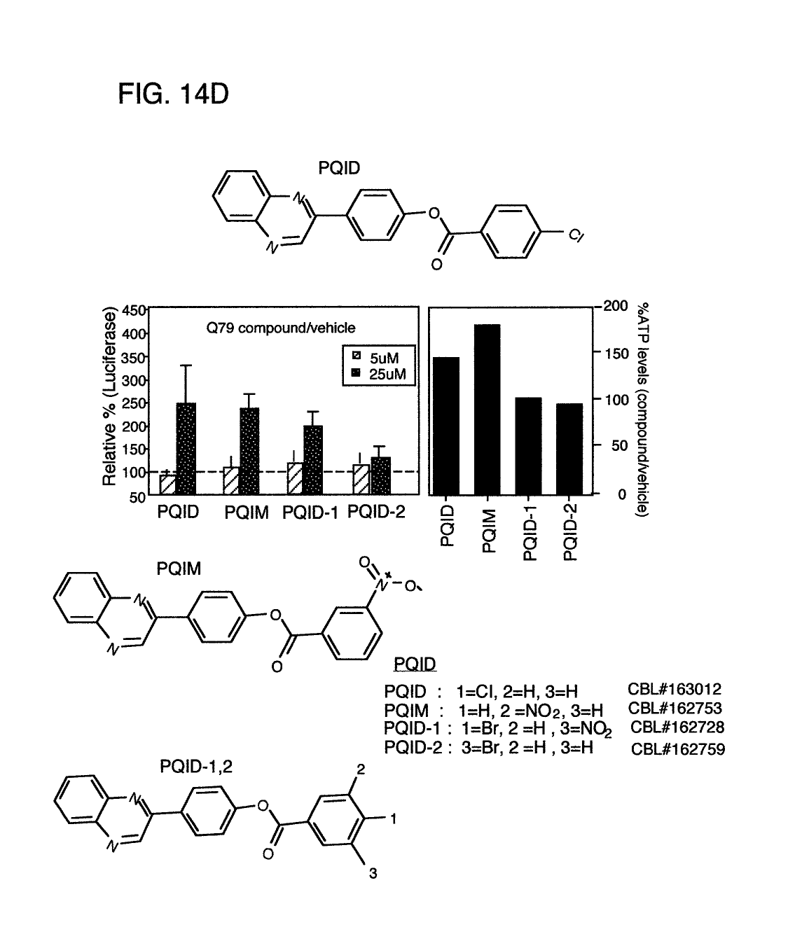

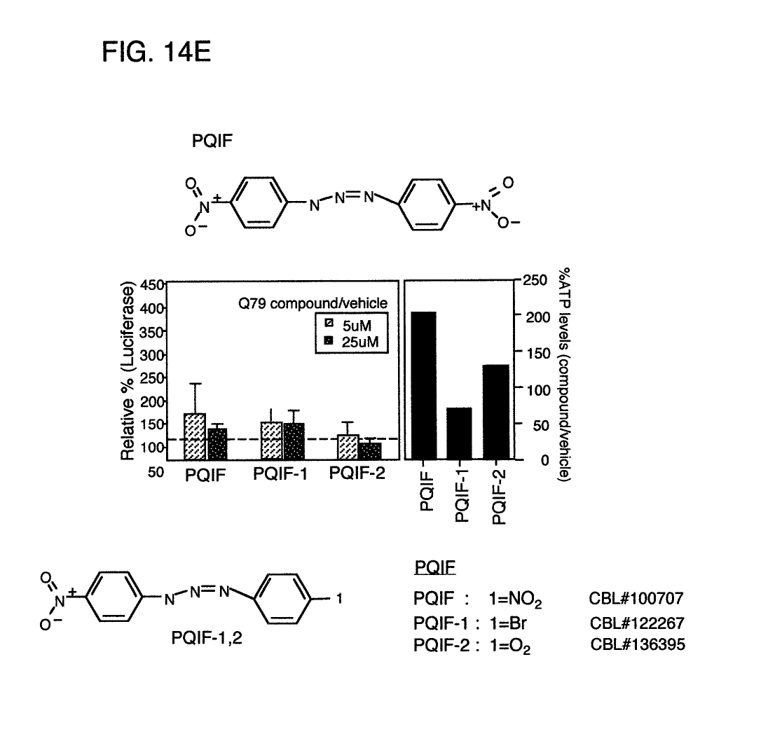

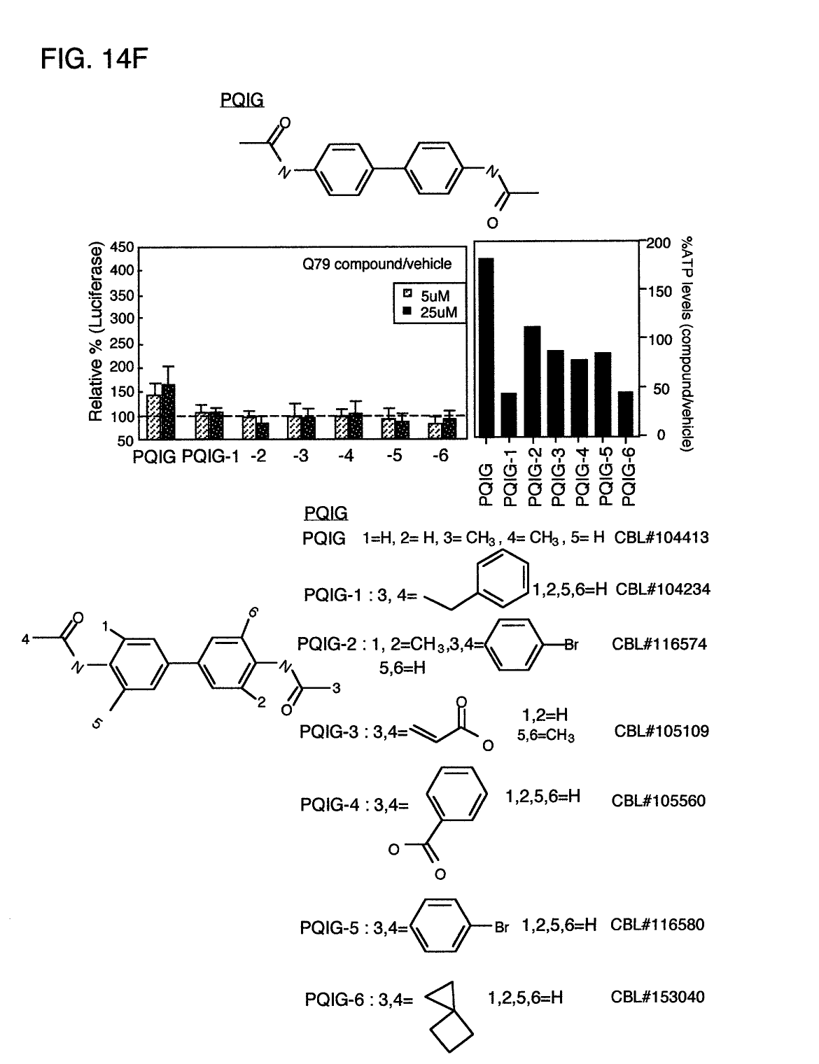

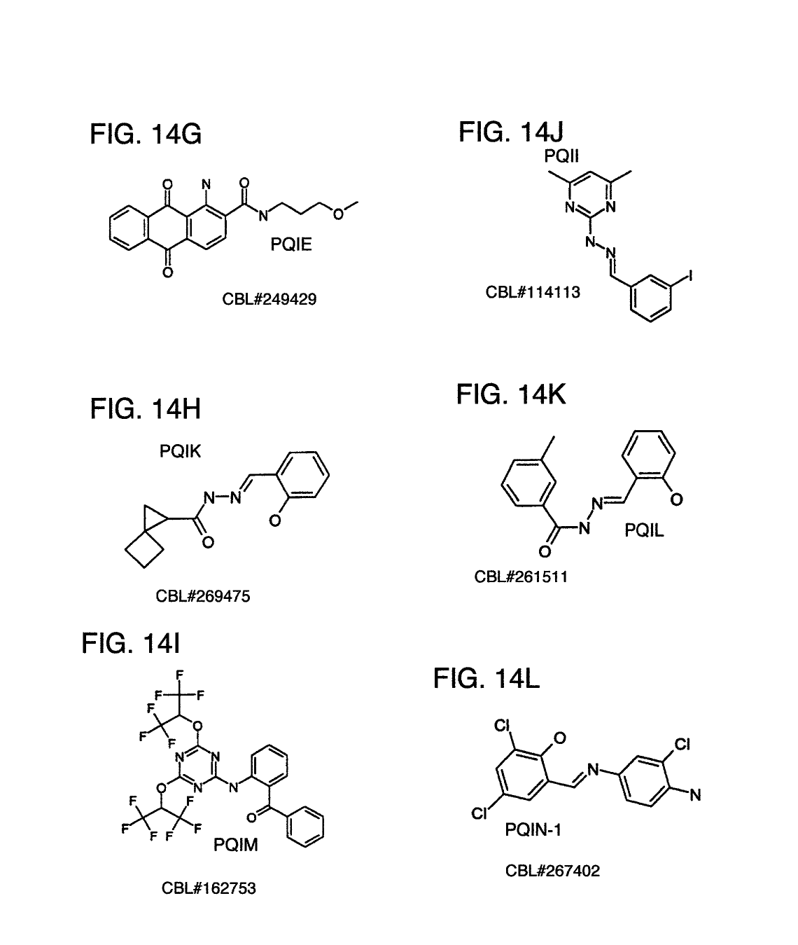

The additional compounds described herein may be structurally modified and subsequently used to decrease cell toxicity or death, or to decrease aggregates formed by amyloidogenic proteins, including polyglutamine aggregates. These derivatives may also be used to treat a condition characterized by the presence of aggregates formed by amyloidogenic polypeptides, or to prophylactically treat a subject at risk for developing a condition characterized by amyloidogenic aggregates. The compounds identified herein as able to decrease cell toxicity or death may be modified to form derivatives, using techniques known in the art. Examples of identified compounds and their derivatives for use in decreasing amyloidogenic aggregates are provided in FIGS. 14A-14L, 15A-15S, and 18A-18O.

The methods of the instant invention may be used to reduce cell toxicity or death or to treat a condition described herein in any mammal, for example, humans, domestic pets, or livestock.

Described herein are methods of inhibiting cell toxicity or death and decreasing protein aggregates or inclusions. Techniques for carrying out each method of the invention are now described in detail, using particular examples. These examples are provided for the purpose of illustrating the invention, and should not be construed as limiting.

### EXAMPLE 1

### Generation of Cells Expressing an Expanded Polyglutamine Repeat

Cells, including neuroblastoma cells, HeLa cells, primary neuronal cells, and primary embryonic neuronal cells were transiently transfected with the plasmid Q79-GFP, encoding 79 glutamines followed by the jellyfish green fluorescent protein (Sanchez et al., supra). Expression of Q79-GFP was observed 48 or 72 hours after transfection by visual inspection of GFP-positive cells using an inverted fluorescence microscope.

### EXAMPLE 2

### Protection of Expanded Polyglutamine-Induced Cell Death by Congo Red

In a cell expressing Q79-GFP polypeptides, polyglutamine aggregates are formed and the cell dies by apoptosis (Sanchez, supra). Cell viability was determined by morphological criteria characteristic of apoptosis, including cell blebbing, shrinking. Total cell lysates and tissue homogenates were obtained using Buffer A: 1% triton in PBS, containing 10 μg/ml DNAse and protease inhibitors; leupeptin 20 μg/ml, aprotinin 20 u/ml, and PMSF 100 mM. The proteins were separated by 12% SDS/PAGE and transferred to PVDF Immobilon membrane (Millipore, Inc). Antibodies used included rabbit polyclonal antibodies; EM48 (Li et al., Mol. Neurobiol. 20:111-124, 2000) anti-heat shock proteins 40 and 70 (Stressgen, Co.), and rat monoclonal anti-caspase-8 antibody.

These effects were determined by visual inspection of GFP-positive cells under an inverted fluorescence microscope. In addition, the occurrence of apoptosis of these transfected cell samples was confirmed by staining the cells with trypan blue, propidium iodide, or Hoescht 33342 to examine the cellular or nuclear morphology.

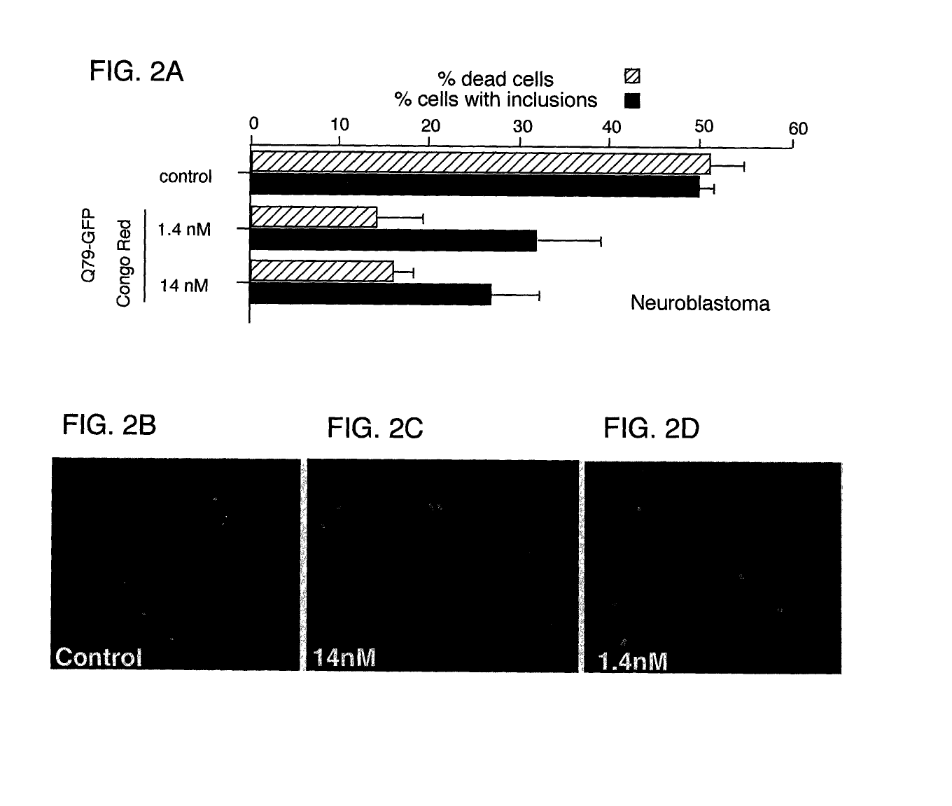

To examine the mechanism by which Congo red inhibits expanded polyglutamine repeat induced cell death, we tested whether Congo red could inhibit polyglutamine oligomerization in cultured neuroblastoma (SHSY) cells transfected with Q79-GFP (FIG. 2A). Cells expressing Q79-GFP were incubated in cell culture media containing Congo red and evaluated for the effect of Congo red on polyglutamine aggregation and cell death. Congo red concentrations of 1.5 to 100 nM inhibited the formation of polyglutamine aggregates and significantly reduced cell death (FIGS. 2A-2D). These results indicate that Congo red may be used to inhibit cell death induced by polyglutamine aggregates. The cells were then scored the presence of visible oligomers by fluorescence microscopy. Cells in which oligomers were not detected at this level of resolution were scored as cells lacking polyglutamine aggregates. Thus, the ability of Congo red to inhibit expanded polyglutamine repeat induced cell death cytotoxicity correlated with its ability to disrupt polyglutamine aggregate formation.

### EXAMPLE 3

### The Effect of Congo Red on the Decrease of Pre-formed Polyglutamine Aggregates in Vitro

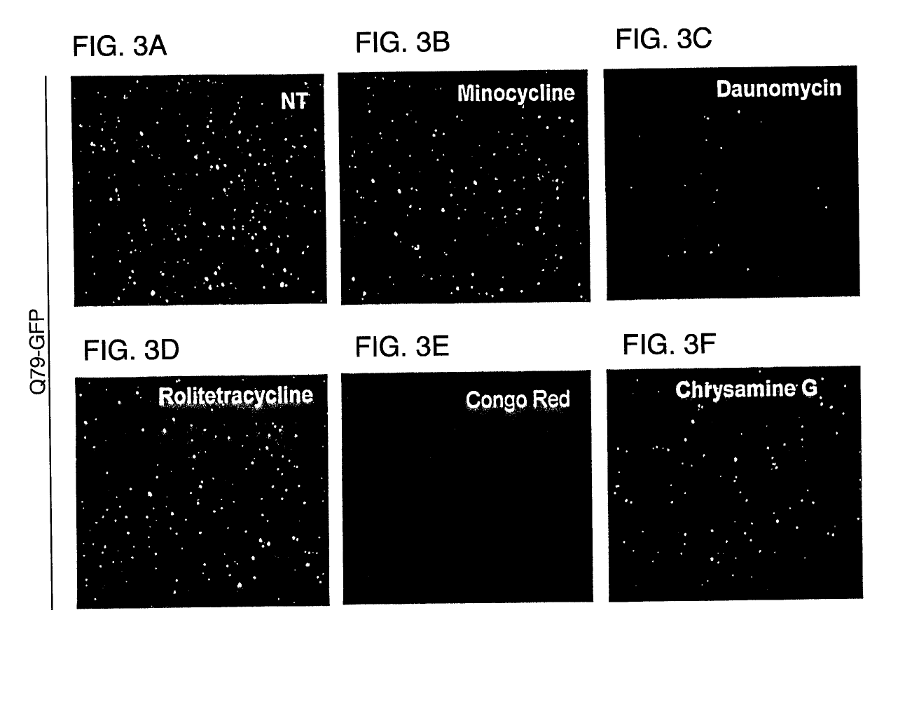

Q79-GFP was expressed and purified from bacteria using standard molecular biology techniques. These fusion proteins form aggregates in vitro which can be viewed under an inverted fluorescence microscope. The Q79-GFP aggregates were administered Congo red (1 nM to 100 μM) and the ability of Congo red to decrease the aggregate was visually assessed. Concentrations of 1 nM to 100 μM) of Congo red decreased the pre-formed polyglutamine aggregates (FIG. 3E).

This assay was also carried out on Q79-GFP aggregates that were administered compounds known to disrupt other types of aggregates, for example, beta-amyloid fibril aggregates. The Q79-GFP aggregates were administered Iota-carrageenan, dextran, minocycline, rolitetracycline, or Chrysamine G Congo red (100 μM each), and visually examined for the effect of each compound on disruption of the aggregate, as well as the density and size of the aggregates. As shown in FIGS. 3A-3D and 3F, dissolution or disruption of pre-formed expanded polyglutamine repeat aggregates were detected only after Congo red addition and daunomycin addition. In further experiments to test for the compound toxicity in cells daunomycin was found to be highly toxic to cells, unlike Congo red which was not toxic even to primary neurons.

### EXAMPLE 4

### The Effect of Various Compounds on Polyglutamine-induced Cell Death

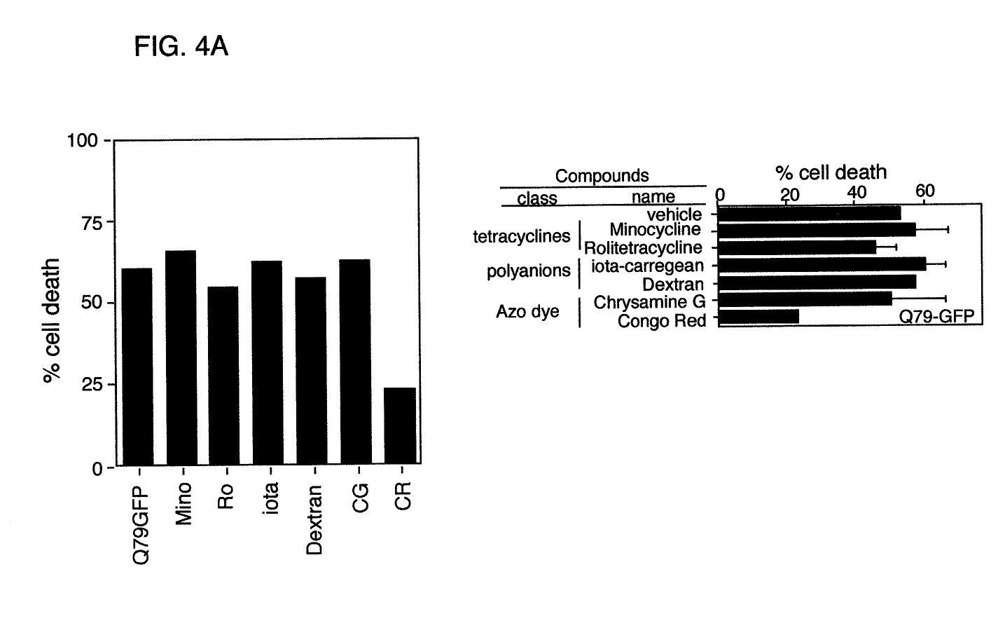

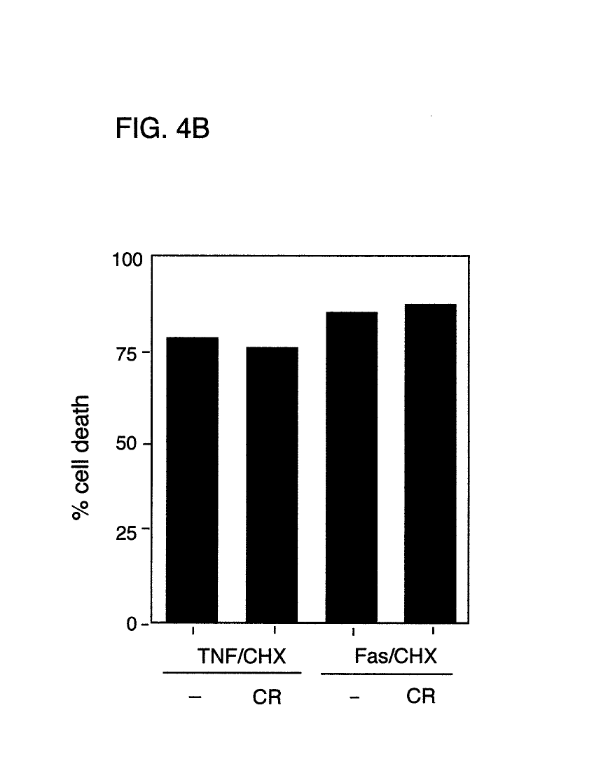

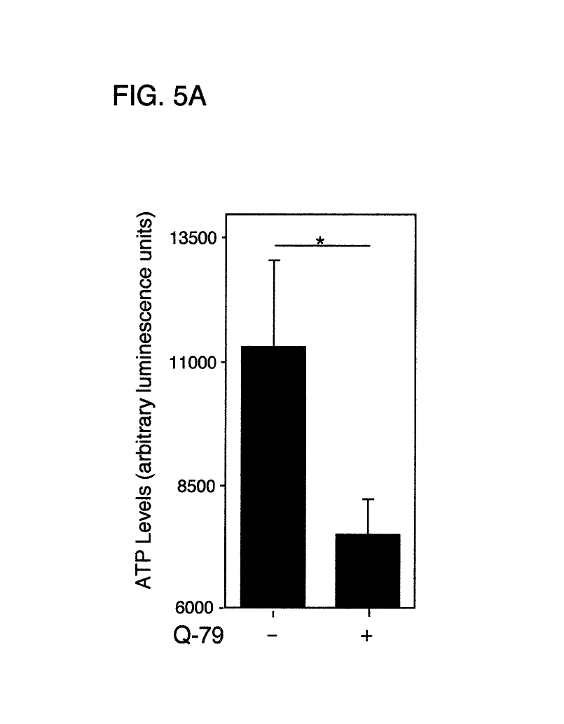

Compounds that disrupt other types of aggregates, for example, amyloid fibril aggregates were tested for their ability to disrupt pre-formed polyglutamine aggregates. HeLa Cells were transfected with a Q79-GFP construct. Six hours later, Iota-carrageenan, dextran, minocycline, rolitetracycline, and Chrysamine G (1 nM to 100 μM) were each incubated with the cells. Seventy-two hours post-transfection, the cells were evaluated for the occurrence of cell death, by visual inspection under an inverted fluorescence microscope. This assay was repeated twice and the results of these studies are summarized in Table 1 and FIG. 4A.

TABLE 1
The Effect of Various Compounds on Polyglutamine-induced Cell Death

| Compound | Polyglutamine-induced Cell Death |
|---|---|
| no treatment | +++ |
| Congo red | + |
| minocycline | +++ |
| rolitetracycline | +++ |
| Chrysamine G | +++ |
| iota-carrageenan | +++ |
| dextran | +++ |

Each + indicates approximately 20% cell death.

These results indicate that while many compounds protect against cell death in a cell containing other types of aggregates, only Congo red effectively protects cells containing polyglutamine aggregates from death.

The effect of Congo red on the induction of cell death by receptor-mediated pathways was also evaluated. Once again, HeLa cells were transfected with the Q79-GFP plasmid, and six hours later were administered Tumor Necrosis Factor-alpha (TNF-α) or anti-FasR in the presence of cycloheximide (CHX). The samples were then incubated with or without Congo red (100 μM). The amount of cell death was measured 72 hours after exposure to the compounds (FIG. 4B). The results of these studies indicates that Congo red does not alter the induction of cell death by receptor-mediated pathways, for example, those stimulated by TNF-α or anti-FasR. These results also demonstrate that Congo red has a very specific mechanism of action, and that Congo red does not contribute to the cytotoxicity of cells in response to inflammatory cytokines, indicating that this compound will not invoke harmful inflammatory side effects.

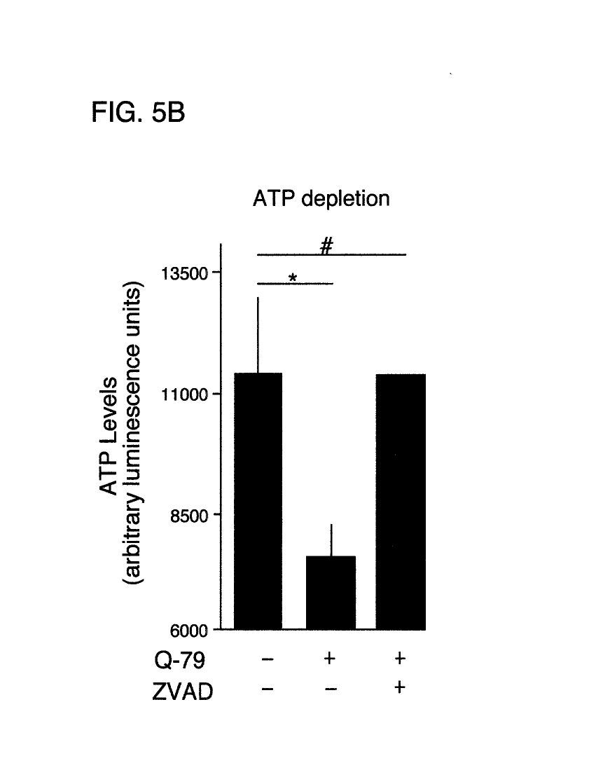

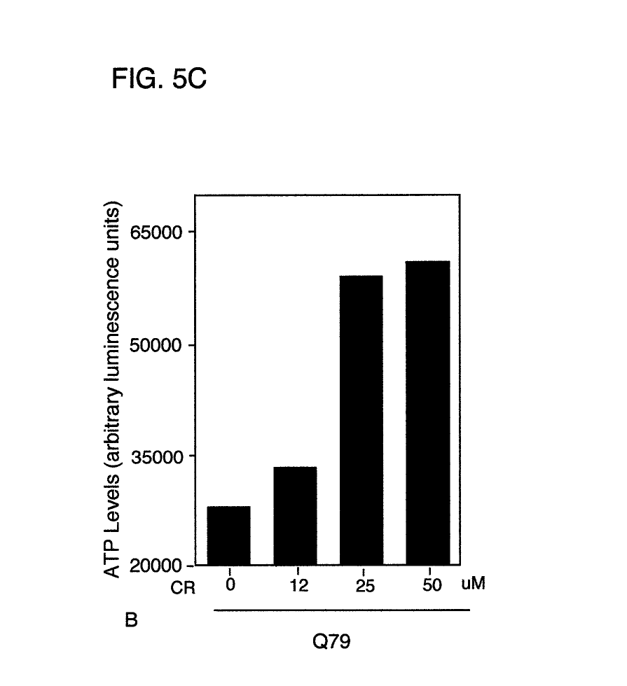

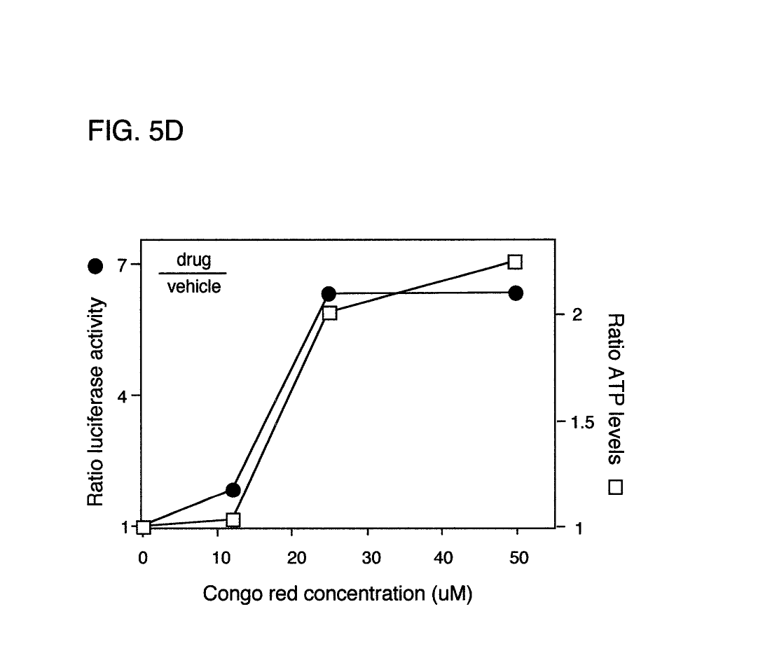

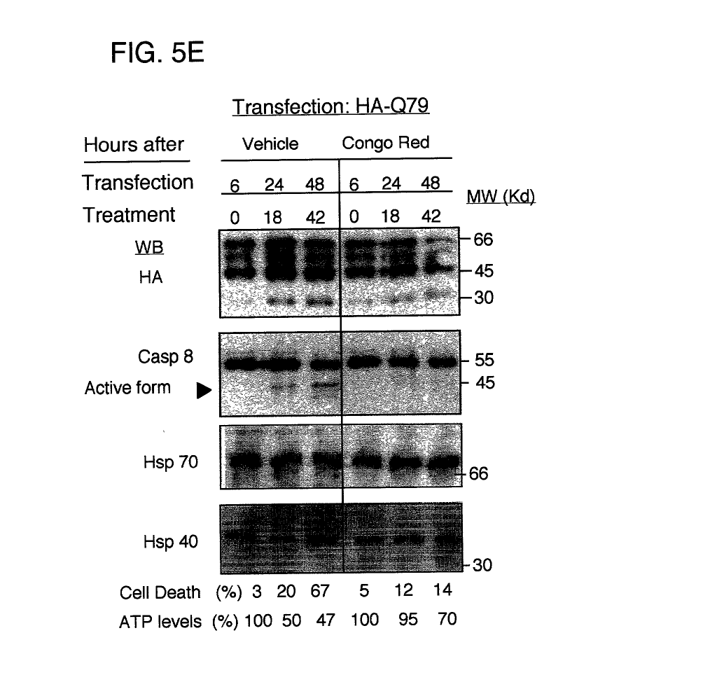

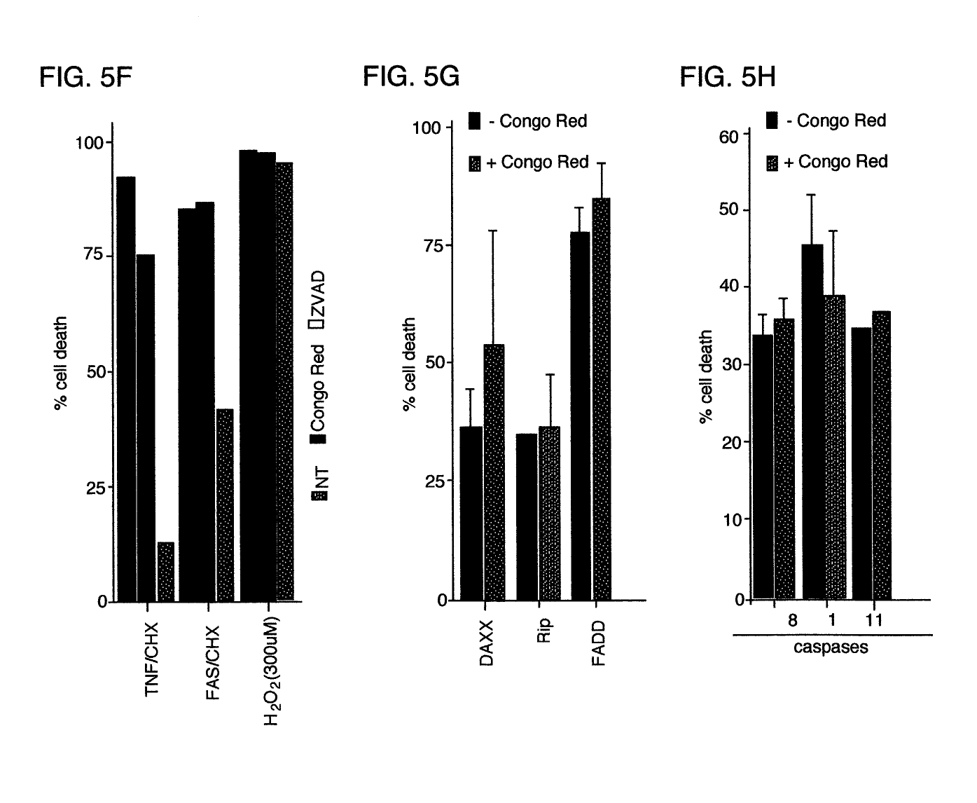

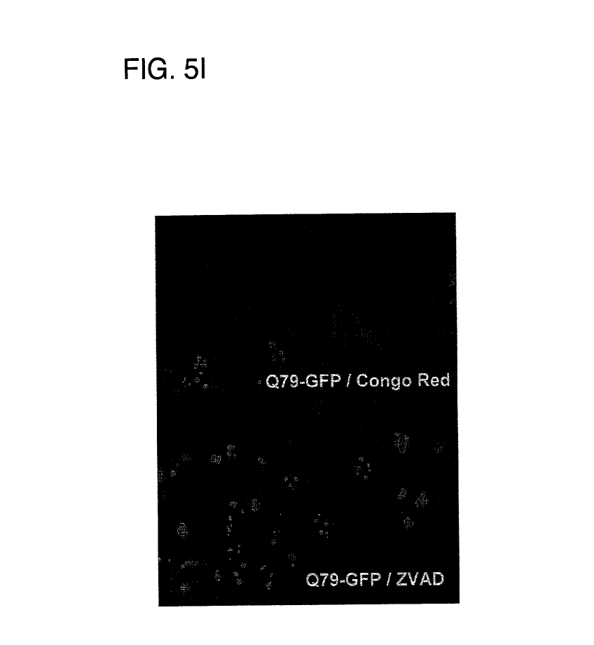

We also investigated whether Congo red prevented other cellular events during cell death, including ATP depletion, caspase activation, and protein synthesis inhibition. HeLa cells were transfected with the Q79-GFP construct. Forty-eight hours later, the viability of cells expressing expanded polyglutamine repeats was determined by measuring cellular ATP levels, suing an ATPLite™ kit, according to the directions provided by the manufacturer (Packard, Co.) (FIG. 5A). Cells transfected with the Q79-GFP construct displayed a lower level of cell viability than the control cells (which do not overexpress expanded polyglutamine repeats), indicating that the expanded polyglutamine repeat was toxic to the cell.

Changes in energy metabolism have been detected in brains of pre-symptomatic HD animal models, and in pre-symptomatic patients. We detected a pronounced decline in ATP levels upon expanded polyglutamine expression in cells as early as 24 hours. This ATP depletion, however, was inhibited by the addition of ZVAD, a pan-caspase inhibitor, suggesting that these changes in energy metabolism leading to reduction of ATP levels are downstream of caspase activation (FIG. 5B). Expression of Q79-GFP in HeLa cells transfected with Q79-GFP and subsequently treated with Congo red (100 μM), or ZVAD (100 μM), is shown in FIG. 51. The Congo red-treated cells displayed fewer polyglutamine aggregates.

To determine the effect of Congo red (100 μM) on polyglutamine-induced cell toxicity. HeLa cells were transiently transfected with Q79-GFP, and six hours later were administered various concentrations of Congo red. Cell viability was assayed 72 hours after transfection, by measuring the level of ATP in each sample. ATP levels in cells expressing Q79 were determined by using the ATPLite™ kit as recommended by the manufacturer (Packard, Inc.). Briefly, cells were transfected with Q79 and treated 6 hours after transfection. Forty-two hours later, the levels of ATP were determined. (FIG. 5C). The results of this study showed that Congo red begins to protect cells from polyglutamine repeat-induced cell death at a concentration of 12 μM, and is very effective at protecting cells at concentrations of 25 or 50 μM.

### EXAMPLE 6

### The Effect of Congo Red on Protein Synthesis in HeLa Cells Expressing Expanded Polyglutamine Repeats

Inhibition of most protein synthesis is an important event in cell death. To determine if Congo red inhibits the loss of protein synthesis in polyglutamine expressing cells, HeLa cells were co-transfected with a luciferase construct together with Q79-GFP. Protein synthesis was determined by measuring luciferase activity 48 hours after transfection. Expression of Q79-GFP resulted in the significant loss of luciferase activity. Treatment of the cells with Congo red prevented the loss of luciferase activity with the same dose response curve as that of inhibition of ATP loss (FIG. 5D).

### EXAMPLE 7

### The Effect of Congo Red on the Activation of Caspase-8 in HeLa Cells Expressing Expanded Polyglutamine Repeats

Expanded polyglutamine repeats recruit and activate caspase-8 (Sanchez et al., supra). We tested if the treatment of cells expressing expanded polyglutamine repeats with Congo red inhibits the activation of caspase-8. HeLa cells were transfected with a hemagglutinin-tagged (HA) Q79-GFP construct in the presence or absence of Congo red and the cell lysates were analyzed by Western blot analysis for the expression of caspase-8, or for expression of HA. The appearance of a 45 kilodalton active caspase-8 fragment was detected at 24 hours after transfection, which was before significant loss of cytoplasmic membrane integrity and morphological changes occurred, and near the time of ATP loss (FIG. 5E). Treatment of the cells with Congo red completely inhibited the appearance of active caspase-8 and significantly inhibited ATP loss and cell death, as indicated by morphology.

### EXAMPLE 8

### The Effect of Congo Red on the Expression of Chaperone Proteins in HeLa Cells Expressing Expanded Polyglutamine Repeats

The expression of chaperone proteins, for example, HSP40 and HSP70 has been shown to inhibit cytotoxicity, and appears to alter the properties of polyglutamine oligomers. We examined whether the destabilizing effect of Congo red on polyglutamine oligomers in HeLa cells expressing expanded polyglutamine repeats was caused indirectly through increased expression of endogenous chaperones (FIG. 5E). Lysates from Congo red-treated or control cells expressing expanded polyglutamine repeats (HA-Q79) were separated by SDS-PAGE and probed with anti-HSP70, anti-HSP40, and anti-HA antibodies to detect polyglutamine repeats. The results of these studies indicated that Congo red treatment of Q79-expressing cells did not induce the expression of HSP40 or HSP70; thus, Congo red is unlikely to act indirectly through induction of chaperone proteins.

### EXAMPLE 9

### The Effect of Congo Red on the Function of Apoptotic Machinery in HeLa Cells Expressing Expanded Polyglutamine Repeats

To examine the possibility that Congo red directly inhibits molecules involved in modulating apoptotic, in addition to or associated with its ability to inhibit amyloid-like polyglutamine repeat oligomer formation, we tested the effect of Congo red on cells induced to undergo cell death under various conditions. HeLa cells expressing Q79 expanded polyglutamine repeats were treated with 100 μM Congo red, or 100 μM ZVAD (a positive control) for one hour prior to the addition of the apoptosis-inducing agent, and cell death was determined 48 hours later. No effect of Congo red was detected when cell death was induced by treatment with TNF-α/CHX, Fas/CHX, or H₂O₂, although ZVAD was effective in inhibiting both TNF-α- and Fas-induced apoptosis (FIG. 5F). We also tested a number of adapter proteins involved in apoptosis, including RIP, FADD, and Daxx, which have been shown to form oligomers and induce cell death when over-expressed. Treatment of the cells with Congo red had no effect on apoptosis induced by overexpression of RIP, FADD, or Daxx (FIG. 5G), suggesting that Congo red does not inhibit protein oligomerization in general. Finally we tested whether Congo red can inhibit apoptosis induced by overexpression of caspase-1, -8, or -11. Congo red had no effect on caspase induced cell death (FIG. 5H). These data confirm that Congo red does not target general components of the apoptotic pathway.

### EXAMPLE 10

### Selective Inhibition of Expanded Polyglutamine Repeat Oligomerization by Congo Red

To determine if Congo red and its derivatives directly bind to expanded polyglutamine aggregates, we designed a chemical absorption assay. This assay measures the remaining Congo red in the supernatant after incubating the supernatant with GST-tagged polyglutamine beads, and removing the GST-polyglutamine beads by centrifugation. This assay was carried out as follows. Recombinant protein GST-Q19, GST-Q62, and GST-Q81 (containing 19, 62, and 81 polyglutamines, respectively) was purified from E. coli by glutathione beads. After several washes with PBS containing protease inhibitors, the beads were allowed to settle to the bottom of the tube at 24° C. for 10 minutes. The polyglutamine-bound beads were aliquoted and diluted with two volumes of 10 μg/ml of Congo red in PBS. The beads were mixed and centrifuged at 15,000 rpm for 2 minutes and the supernatant was removed and diluted 1:100. The percentage absorbance of the compound remaining in the supernatant after absorption with the GST-polyQ beads was then detected. In addition, Congo red- or PBS-only treated polyglutamine polypeptide bead pellets were washed three times with 100 volumes of PBS and then the protein was separated by 12% polyacrylamide gels and immunoblotted as mentioned above. Some blots were used for immunostaining with anti-polyglutamine antibody (EM48). Other blots were left unstained to visualize the red color of Congo red that entered the gel along with the polyglutamine protein.

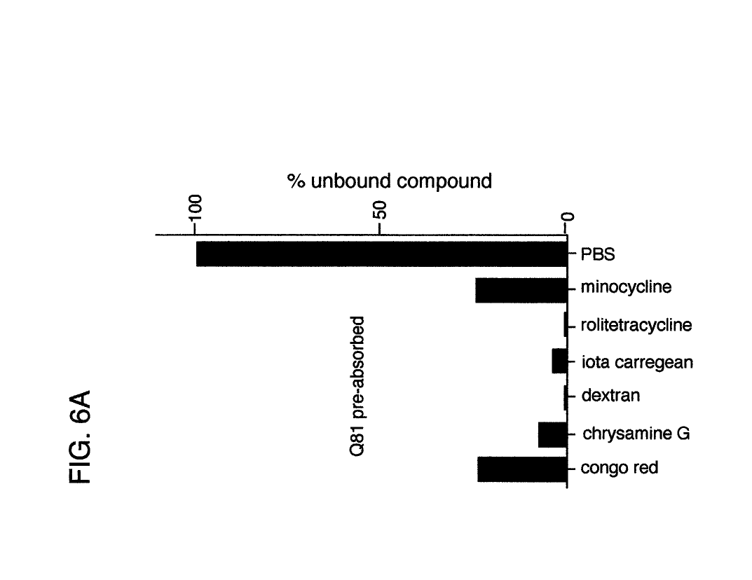

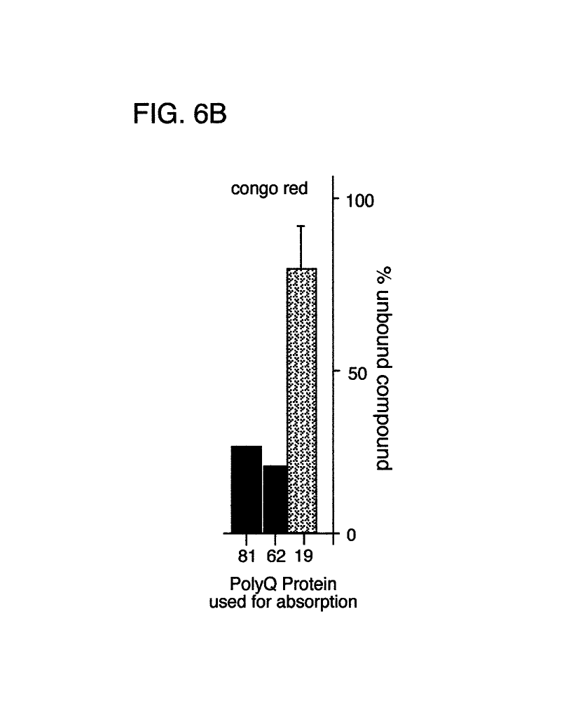

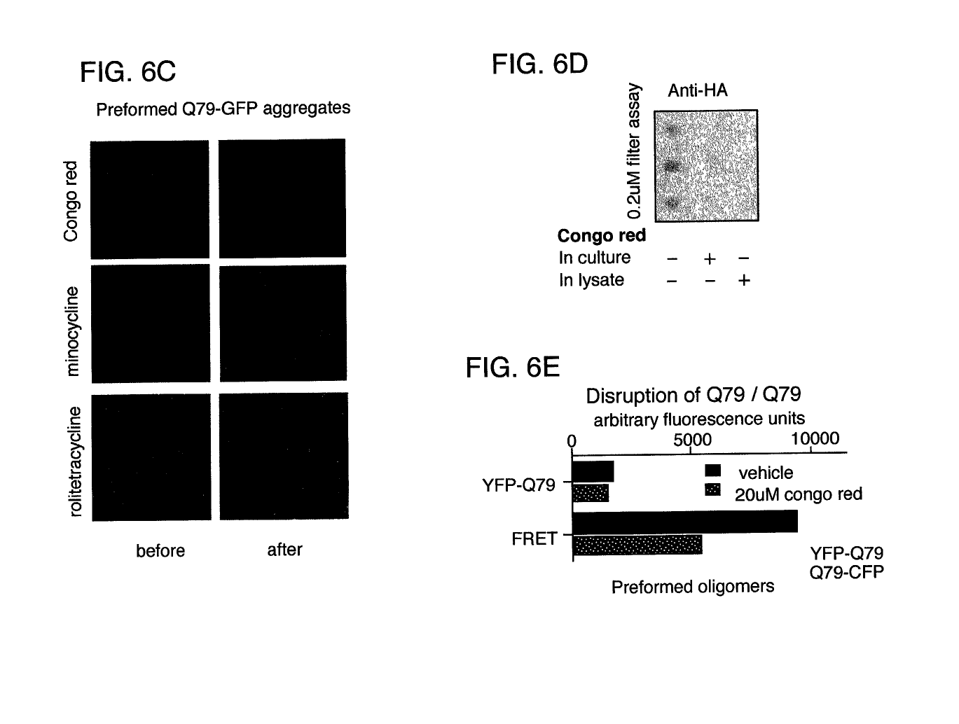

The results of the above-described binding assay showed that Congo red had a significantly higher affinity for expanded polyglutamine repeats that were 81 or 62 polyglutamines in length, compared to polyglutamine repeat containing 19 glutamines (at equal protein concentrations, based on Coomassie blue staining) (FIG. 6A). To determine if binding to polyglutamine is sufficient to inhibit the aggregate formation, we compared the ability of minocycline, rolitetracycline, iota carrageenan, dextran, chrysamine G, and Congo red to bind to 81-repeat polyglutamine polypeptide by the chemical absorption assay described above (FIG. 6B). The results indicated that each of these compounds binds to expanded polyglutamines. To examine the relationship of compound binding and its ability to dissolve expanded polyglutamine aggregates, we tested their effects on semi-purified Q79 oligomers from HeLa cells expressing transfected Q79-GFP, viewed using a microscope. Congo red, but none of the other compounds tested, caused the disassembly of pre-formed poly-Q oligomers as indicated by the disappearance of fluorescent aggregates (FIG. 6C).

To further confirm the ability of Congo red to inhibit the formation of polyglutamine aggregates, as well as to disrupt preformed aggregates, we analyzed the state of polyglutamine aggregation by examining equal aliquots of total lysates from Congo-red- or vehicle-treated HA-Q79-GFP expressing cells, or the lysates that were treated with Congo red or vehicle after cell lysis. The cell lysates were passed through a 0.2 μm acetate filter and then visualized by immunostaining with anti-HA antibody (FIG. 6D). Polyglutamine immunoreactivity was detected in the lysates from untreated Q79-GFP transfected cells, indicating that polyglutamine aggregates are large enough to be retained by 0.2 μm filter. The polyglutamine aggregates, however, were undetectable in the cell lysates that were isolated from cells treated with Congo red 6 hours after transfection or in the cell lysates treated with Congo red after cell lysis (FIG. 6D), indicating that Congo red is able to inhibit the formation of polyglutamine aggregates that are larger than 0.2 μm in size in cells, and dissolve the preformed polyglutamine aggregates.

To rule out the possibility that Congo red merely disrupts large polyglutamine aggregates without affecting oligomeric polyglutamine interaction that may be invisible to fluorescence microscopy, we designed a FRET-Q79 assay. In this assay, the expression constructs EYFP-Q79 (enhanced yellow fluorescent protein fused to a polypeptide containing 79 polyglutamines) and Q79-ECFP (enhanced cyan fluorescent protein fused to a polypeptide containing 79 polyglutamines) were co-transfected into HeLa cells. The EYFP-HAQ79 was made by ligation of a SacI/Notl digested insert from HA-Q79 CMX (Sanchez et al., 1999, supra) into the plasmid CFP-C1, described by Sanchez et al. (supra) after Sac1 digestion and blunting. The HindIII/NotI digested inserts from HAQ79-GFPN1 was used to make the HA-Q79-CFP-N1 construct. Either Q79-GFP or Q79-ECFP/EYFP-Q79 expressing HeLa cells were cultured for 48 hours, harvested and lysed.

The lysates were passed through a needle to ensure release of polyglutamine inclusions from the nuclei. The supernatants were centrifuged twice at 4,000 rpm for 30 seconds over a 25% sucrose cushion to remove unlysed nuclei. Polyglutamine aggregates were aliquoted into 384 well plates and treated with 100 Congo red. The fluorescence resonance energy transfer (FRET) was determined from polyglutamine oligomers formed in Q79-ECFP/EYFP-Q79 expressing cells upon addition of PBS or Congo red. Specifically, the interaction of the tagged Q79 polypeptides was detected by immunofluorescence at 488 nm excitation and 527 nm emission for FRET using a Wallach plate reader, and the ratio of the immunofluorescence was determined. Immunofluorescence levels were also detected for each ECFP and EYFP independent of the effect of Congo red on fluorescence emission wavelengths. The FRET ratio was significantly reduced upon incubation with Congo red (FIG. 6E), indicating Congo red disrupts the Q79-Q79 interaction or the polyglutamine oligomerization.

### EXAMPLE 11

### Inhibition of Polyglutamine Oligomerization Prevents the Abnormal Recruitment of Expanded Polyglutamine Interacting Proteins

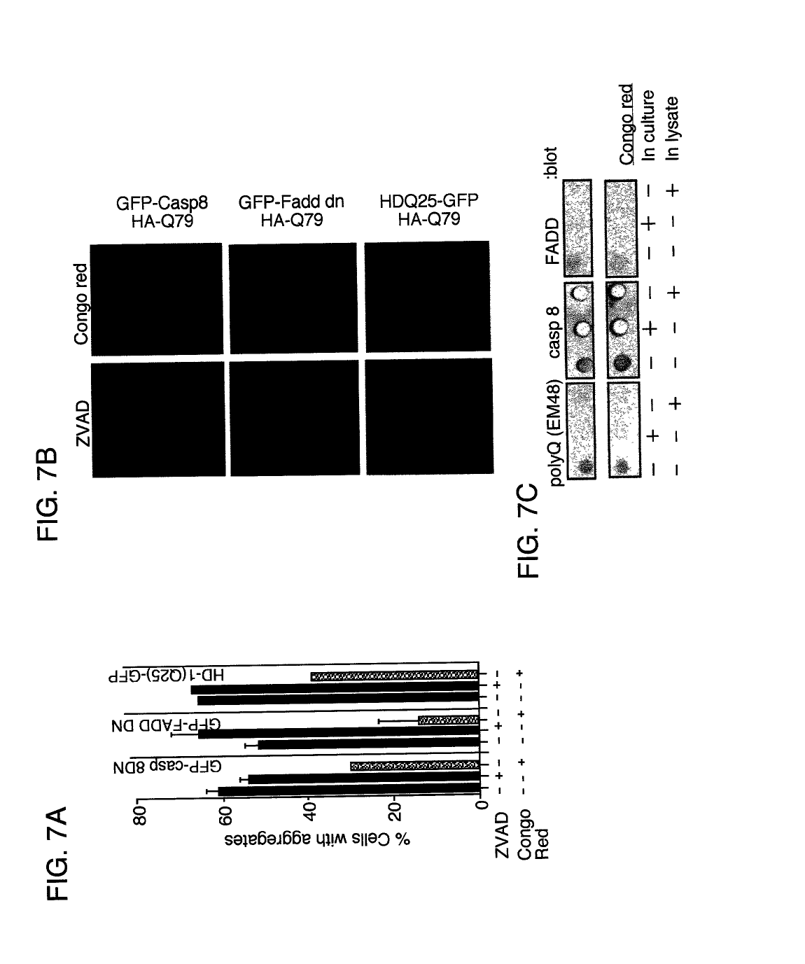

As several proteins, including the death domain of FADD, caspase-8, and proteins containing short polyglutamine repeats, including normal huntingtin protein and transcription factors have been shown to co-localize with oligomerized expanded polyglutamine repeats, we determined if disruption of polyglutamine oligomerization interferes with its interaction with such proteins. We co-transfected HeLa cells with Q-79 and one of the following expression constructs, GFP-FADDdn (GFP fused to the death domain of FADD adapter protein, that generates a dominant negative FADD polypeptide (missing the first 80 amino acids of the full length protein)), GFP-caspase-8dn (GFP-tagged C360S mutant of caspase-8, generating a dominant negative caspase-8 polypeptide; Sanchez et al., supra), or GFP-exon-1 (Q25) (a GFP fusion construct containing the first exon of human huntingtin protein, encoding a 25 polyglutamine stretch; Kazantsev et al., Proc. Natl. Acad. Sci. USA 96:11202-11409, 1999), in the presence of ZVAD or of Congo red (FIGS. 7A and B).

Inhibition of polyglutamine aggregation by Congo red decreased the concurrent recruitment of FADDdn, caspase-8dn, and HD exon-1 (Q25)-GFP. These results confirm that the aggregation of FADD, caspase-8, and wild type huntingtin is not indirectly triggered by the expression of expanded polyglutamine, but rather is dependant upon polyglutamine oligomerization. Thus, this data shows that specific disruption of expanded polyglutamine oligomerization with Congo red is sufficient to prevent the abnormal aggregation of recruited proteins. These results were also confirmed by using the filter assay (FIG. 7C), where FADD and caspase-8 were found in a complex that was retained by 0.2 μm filter in vehicle-treated lysates but not in Congo red-treated lysates of Q79 expressing cells. These results underscore the selectivity of Congo red on oligomeric expanded polyglutamine repeats and its inhibitory effects on subsequent downstream events, including aberrant protein-protein interactions.

### EXAMPLE 12

### Disruption of Oligomerization Promotes the Turnover of Expanded Polyglutamine Repeats

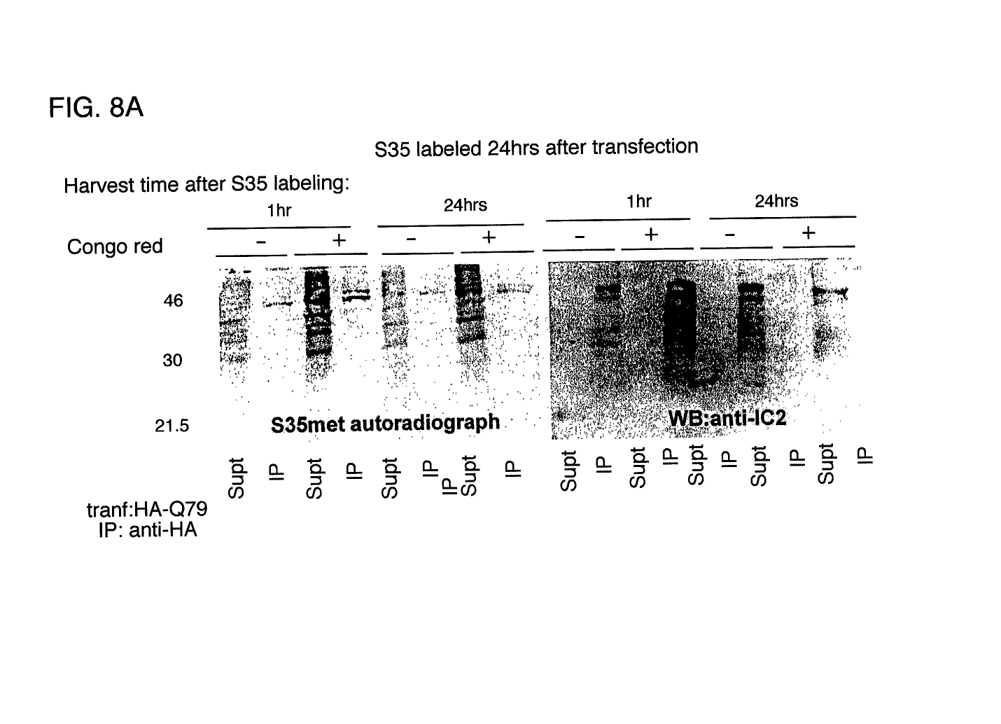

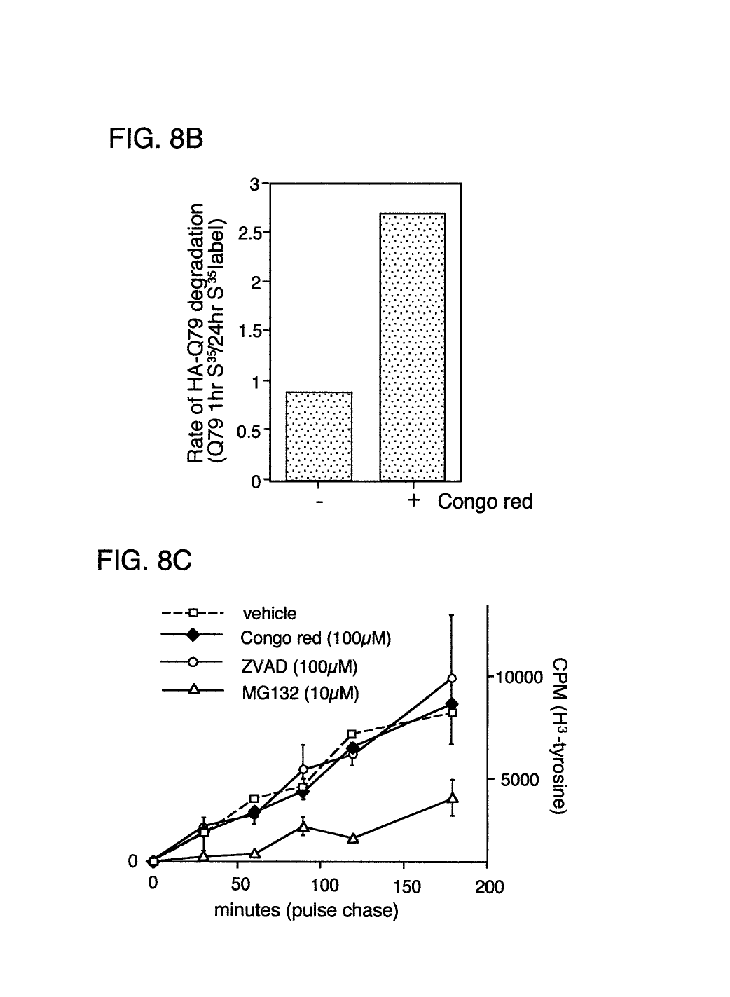

Expanded polyglutamine oligomers accumulate in cells and appear to have a significantly slower turnover rate than that of shorter polyglutamine oligomers. We next determined whether inhibition and disruption of Q79 oligomers changed its turnover kinetics by measuring pulse-S³⁵-labeled polyglutamine levels in the presence or absence of Congo red. Treatment of Q79-expressing HeLa cells with Congo red did not prevent the expression of expanded polyglutamine, as seen one hour after chase, suggesting that Congo red does not affect the synthesis of polyglutamine directly (FIG. 8A). The level of labeled polyglutamine, however, is more than two fold lower in Congo red-treated cells after chase for 24 hours (FIG. 8A and 8B), indicating that S³⁵-labeled Q79 in Congo red-treated cells has at least a two fold higher turnover rate than that of control cells. These data indicate that the turnover rate of polyglutamine can be enhanced by inhibition of its oligomerization, suggesting that changes in its secondary structure or the inhibition of polyglutamine oligomerization may be responsible for its increased turnover or clearance from cells. Treatment of the cells with Congo red, however, did not reduce cellular expanded polyglutamine expression, as indicated by the levels of newly synthesized HA-Q79 (FIG. 5E, top panel), as it only slightly reduced steady state levels compared to that of negative controls as assessed by Western blot analysis (FIG. 5E).

To rule out the possibility that Congo red may have an effect on protein degradation in general, we tested the effect of Congo red on proteosome degradation. Rates of total cellular protein degradation experiments, from cells seeded in 12 well dishes at 2×10⁴, treated with Congo red (100 μM), ZVAD (100 μM), or the proteosome inhibitor MG132 (10 μM) and labeled with 5 μCi/ml of H³-tyrosine for 1 hour. One hundred microliter culture medium aliquots were collected at 0, 30, 60, 90, 120, and 180 minute time points, and the levels of H³-tyrosine were determined using an LS6000 scintillation counter (Beckman, Co).

While protein degradation was inhibited by the proteosome inhibitor MG132, neither Congo red nor ZVAD had any effect on general protein degradation (FIG. 8C). These data indicate that the effect of Congo red on the turnover kinetics of expanded polyglutamine repeats in cells is not due to its effect on general protein turnover, but rather its specific ability to dissolve the expanded polyglutamine aggregate.

### EXAMPLE 13

### The Use of Congo Red in Treating Conditions Associated with Expanded Polyglutamine Repeats

Transgenic R62 mice that express the human Huntington's disease gene (carrying a 139-157 CAG repeat; Mangiarini et al., Cell 87:493-506, 1996; Carter et al., J. Neurosci. 19:3248-3257, 1999; and Levine et al., J. Neurosci. Res. 58:515-532, 1999) were used to examine the effects of Congo red on cell death or toxicity. This animal model expresses the truncated form of the Huntington protein under the endogenous promoter. Polyglutamine inclusions have been observed in muscle tissue in this animal model, and therefore, the model may also serve to determine the link between polyglutamine inclusions in muscle and muscle weakness.

Administration of Congo red (dissolved in PBS) or PBS only (vehicle) to the R62 transgenic mice and controls is performed by two means; intraperitoneal injection (up to 13 mg/kg) every 48 hours for a period of two weeks (which resulted in no obvious drug toxicity), or long term infusion by intraventricular cannula connected to a pump. The latter procedure involved administration of 0.25 μl of the drug (1 mg/ml in 0.4% DMSO) per hour, for a period of at least 28 days. The treatment began at the latter part of the seventh week. When treatment began, although very little motor symptomology was observed, tremors were becoming apparent. Polyglutamine inclusions were readily detected at the beginning of treatment. The effect of Congo red on the polyglutamine-induced toxicity in muscle strength in the animal model R62 was quantified by the use of a rotorod.

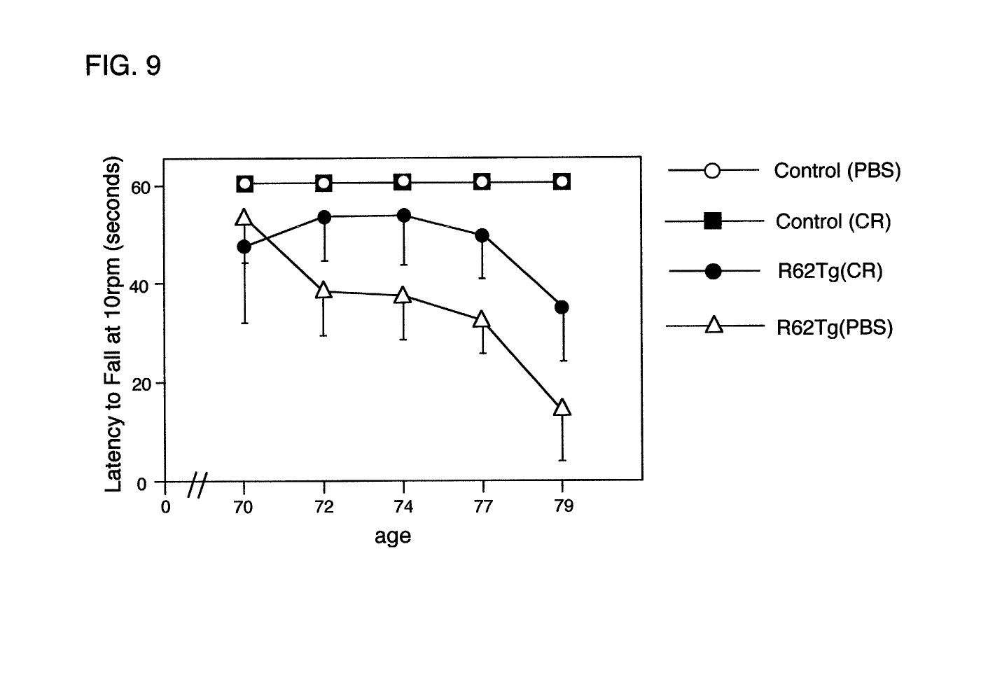

The mice were trained every day for a week after which time the latency to fall from a rotorod at a speed of 10 rpm was measured for each mouse, with a maximum time of 60 seconds per trial, as described by Carter et al., (J. Neurosci. 19:3248-3257, 1999). At least two trials were performed per mouse and the highest time for each mouse was used in calculating the average time per animal group. The results of these studies show that R62Tg mice that received Congo red exhibited a better ability to remain on the rotorod than the R62Tg mice that did not receive Congo red (FIG. 9). These results indicate that Congo red helps to improve muscle strength in animals expressing polyglutamine repeats.

In addition, the ability of Congo red to inhibit the formation as well as to disrupt preformed polyglutamine oligomers allowed us to examine the role of polyglutamine aggregates in neuronal dysfunction and degeneration characteristic of polyglutamine expansion diseases. In these studies, we used the animal model of Huntington's disease (R62), which expresses the truncated form of huntingtin with 139 CAG repeats Mangiarini et al. (supra). We took advantage of our finding that in addition to inhibiting polyglutamine oligomerization, Congo red also disrupts pre-formed oligomers, thus allowing assessment of the impact of Congo red on disease progression after formation of polyglutamine aggregates and the onset of symptoms. R62 transgenic mice and CBAxC57B1/6 F1 wild type littermates were obtained from Jackson Laboratories (Bar Harbor, Me.) at the age of five weeks. The genotype was confirmed by PCR, as described in Mangiarini et al. (supra), and were housed at five per cage in a temperature-controlled environment on a 12 hour light/dark cycle. Mice were anesthetized by intraperitoneal injection of chloral hydrate and osmotic pumps (0.25 μl/hour for 28 days) and the cannula were implanted intracerebroventricularly (Alzet, Co) using predetermined coordinates (AP, −0.5 mm, 1 mm lateral to the bregma). The cannula was secured with the use of dental cement (Henry Schein Co). Congo Red (1 mg/ml) was diluted in PBS devoid of magnesium or calcium with the addition of 0.2% DMSO to increase long-term solubility. For intraperitoneal injections, 0.5 mls of 1 mg/ml of Congo red (in PBS/0.2%DMSO) was used every 48 hours. Congo red was infused into the transgenic mice at a dose of 1 mg per 30 g mouse body weight every 48 hours intraperitoneally (IP), or through a 28 day intracerebroventricular cannula (ICV; 6 μg every 24 hours) placed on the left ventricle at postnatal week nine. Mice were then tested for motor performance and coordination beginning two days after the first treatment using a rotorod at 10 rpm for a maximum of 210 seconds, as described by R. J. Carter et al. (supra). Two trials, three times a week, were performed in a blinded manner. Mice were treated following one week of daily training. As a control, mice were also treated with vehicle medium (PBS with 0.2% DMSO).

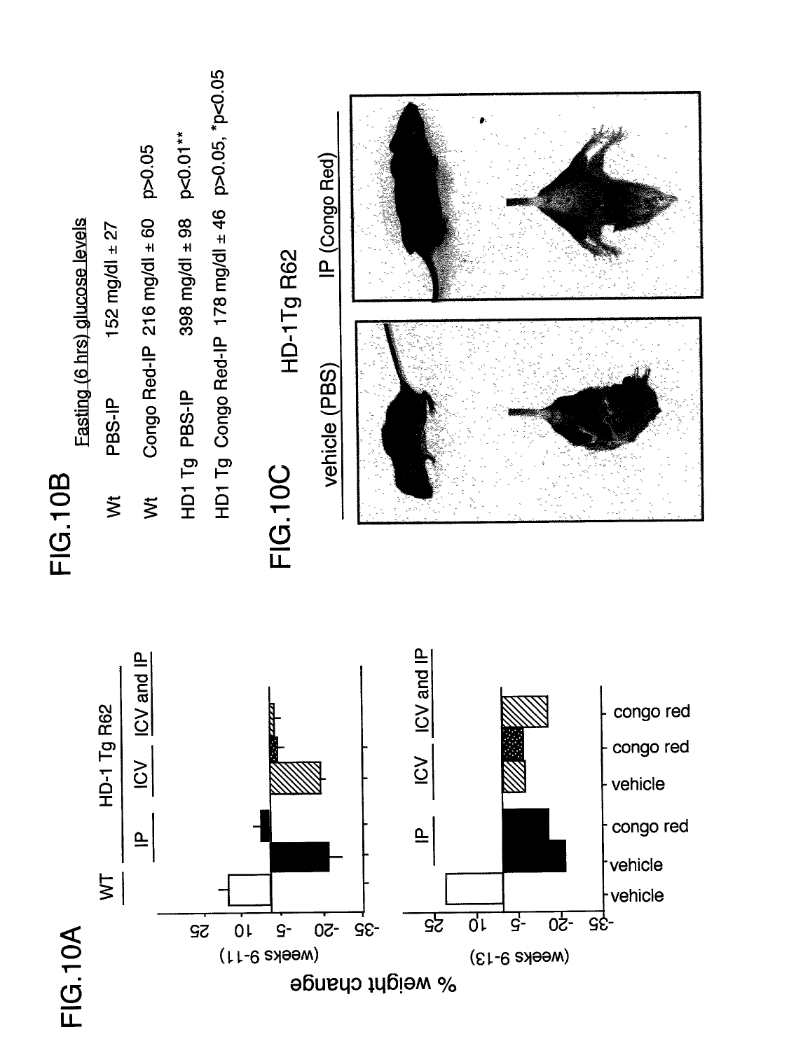

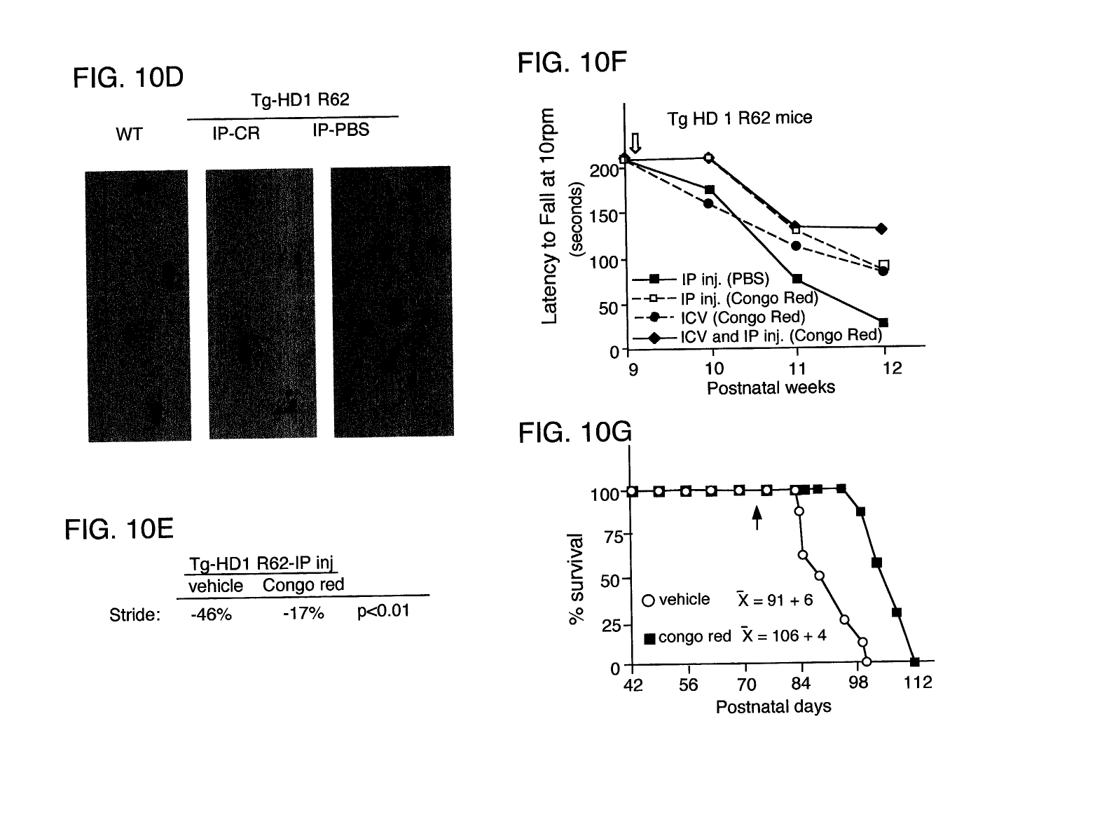

We investigated the effect of the polyglutamine oligomer inhibitor Congo red on weight loss, motor performance, and coordination and survival (FIGS. 10A-10G). Infusion of Congo red of the same doses described above either intraperitoneally (IP) or intracerebroventically (ICV) resulted in no gross morphological abnormalities or induction of any obvious symptoms in normal wild type mice or pre-or post-symptomatic R62 mice.

One of the common features of Huntington's disease is severe weight loss, proposed to be due to metabolic defects as a part of the systemic pathology of the expanded polyglutamine repeat diseases. We monitored the body weight of mice before and after treatment (FIG. 10A). While no change in body weight was detected in Congo red-treated wild-type mice, compared to vehicle treated wild-type mice from 9 to 13 weeks, the severe loss of body weight observed in the expanded polyglutamine repeat expressing mice was significantly ameliorated after Congo red treatment by an ICV or IP route of delivery (FIG. 10A).

R62 mice also develop severe diabetes due to the presence of polyglutamine aggregates in the pancreas. Prior to paraformaldehyde perfusion, blood was obtained intracardially from the right ventricle of anesthetized mice. Collected blood was incubated for 6 hours at 4° C. and centrifuged at 4,000 rpm for 10 minutes. The levels of glucose in the blood after 6 hours of fasting were measured from the serum using the Accu-Check strip kit (Roche, Co.).

Congo red treatment also reduced the fasting glucose levels in blood in HD transgenic mice compared to the normal levels in the wild type (FIG. 10B). Thus, the treatment of Congo red is effective against peripheral symptoms of R62 mice.

The initial abnormal neurological signs of R62 mice include dyskinesia of the hindlimbs when mice were suspended by the tail, and irregular gait. The treatment of mice with Congo red significantly inhibited the dyskinesia of the hindlimbs (FIG. 10C) and preserved the normal gait (FIG. 10D) and stride length (FIG. 10E). The effect of Congo red on motor performance in R62 mice was also assessed by rotarod studies. R62 mice were trained to stay on the rod at 10 rpm for a maximum of 210 seconds at 9 weeks of age. The motor performance of Congo red treated mice was preserved, while the motor function of the PBS-treated control mice continued deteriorating (FIG. 10F). Congo red also significantly prolonged the life span of R62 mice; Congo red-treated R62 mice have a mean survival length of 106 days, whereas control mice survive 91 days (FIG. 10G).

To test whether Congo red can disrupt and inhibit the formation of expanded polyglutamine oligomers in vivo, we analyzed the brain samples from control- and Congo red-treated R62 mice. Mice were anesthetized with isoflurane, perfused intracardially with 4% paraformaldehyde in phosphate buffered saline (pH 7.4). The brain was removed and washed several times in PBS before overnight incubation in PBS containing 30% sucrose, and was then embedded in OTC (Sigma, MO.).

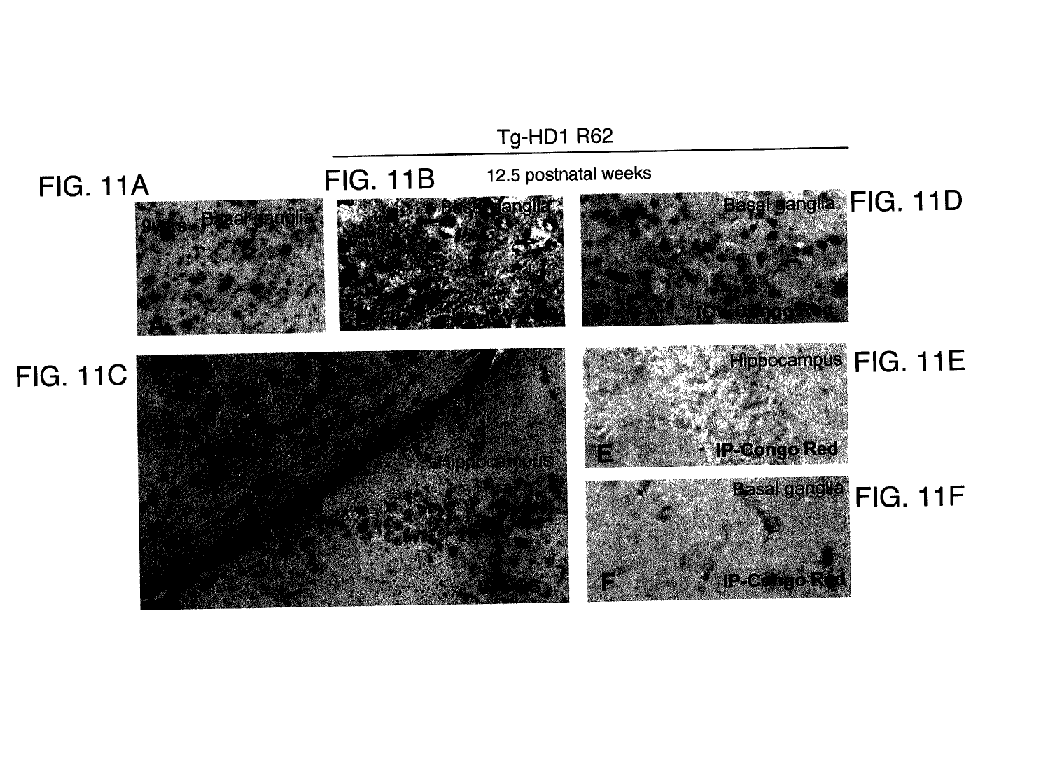

Cryostat frozen sections were washed in PBS, and non-specific binding was blocked with 5% normal goat serum in PBS containing 0.1% Triton. Sections were stained using an anti-EM48 antibody at a 1:1000 dilution, and immunoreactivity was detected using the ABC kit, as described by the manufacturer (Vector, Co.). Some slides were lightly counter stained with hematoxylin. The basal ganglia of 9 week old untreated R62 mice exhibited extensive EM48 positive polyglutamine aggregates (FIG. 11A). Extensive clearance of expanded polyglutamine repeats was observed in the basal ganglia of Congo red-treated 12.5 weeks old, but not in the vehicle-treated mice (FIGS. 11B-11D). A reduction of EM48 positive polyglutamine aggregates upon Congo red treatment was also observed in the cortex and hippocampus (FIGS. 11E and 11F). These results demonstrate that Congo red acts in vivo to disrupt preformed polyglutamine aggregates, as well as to inhibit the formation of new polyglutamine aggregates.

### EXAMPLE 14

### High Throughput Method for Detecting Compounds that Decrease Cell Toxicity or Death

A high-throughput method for identifying compounds that decrease cell toxicity or death has been designed. In this method, cells were seeded at 2×10⁵ cells in 100 mm dishes. The next day the plated cells were transiently transfected with a plasmid encoding an expanded polyglutamine repeat, for example, Q79. Three hours later, transfection efficiency was assessed. Plates containing cells having 10-20% transfection efficiency at this 3 hour time point were trypsinized and re-plated at 600 or at 1500 cells per well in 384 well plates with clear bottoms. After 6 hours, the candidate compounds were added to the multi-well plates. The cells were cultured for 48 hours and ATP levels were determined using an ATPLite™ kit. Data was then expressed in either arbitrary units, or as relative ratio of ATP levels in Q79 transfected cells in compound-treated relative to vehicle-treated samples.

It is understood that any other desired amyloidogenic protein or cell type can be used in this high-throughput assay. To test the effect of a candidate compound on a cell expressing any other amyloidogenic protein, the desired polypeptide is expressed in the desired cell type, using standard methods, for example, as described by Ausubel et al. (supra), and the assay is performed as described above. In addition, methods for expressing amyloidogenic proteins in cells are well known in the art.

### EXAMPLE 15

### Derivatives of Congo Red

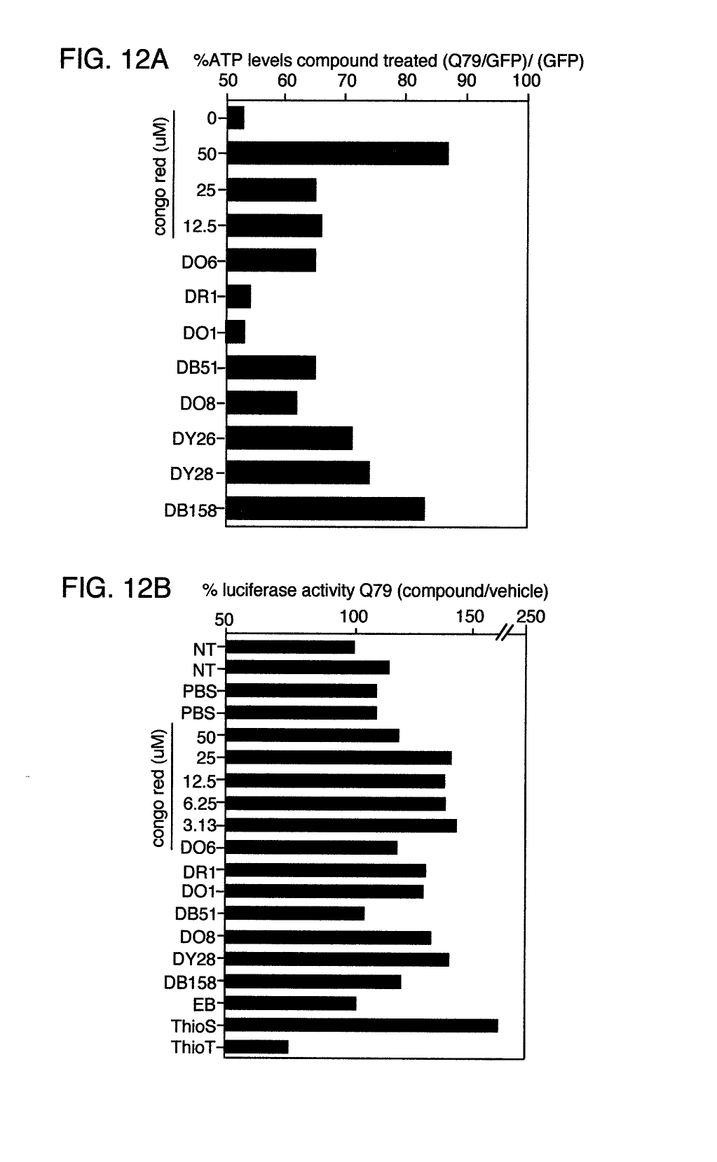

Derivatives of Congo red (commercially available from Sigma/Aldrich) were tested for their ability to alter cell toxicity and death by measuring ATP depletion and luciferase expression. The assay was carried out as described above for the high-throughput identification of compounds. Briefly, HeLa cells were transiently transfected with a Q79/GFP plasmid and were subsequently treated with various concentrations of Congo red, or 25 μM of each of Direct Orange 6, Direct Red 1, Direct Orange 1, Direct Black 51, Direct Orange 8, Direct Yellow 26, Direct Yellow 28, or Direct Blue 158. Drug treatment continued for 6 hours and then 48 hours later, ATP levels were measured in these cells and compared to the ATP levels in cells that were transiently transfected with GFP only, and also received the compound. As shown in FIG. 12A, a number of the Congo red derivatives protected cells from ATP depletion at a level similar to that of Congo red.

This experiment was repeated, using HeLa cells transiently co-transfected with Q79 and luciferase constructs. The drug treatment conditions were the same as described above in the ATP assay. Again, the cells were treated with various concentrations of Congo red, or 25 μM each or Direct Orange 6, Direct Red 1, Direct Orange 1, Direct Black 51, Direct Orange 8, Direct Yellow 26, Direct Yellow 28, or Direct Blue 158. The effect of the compounds on protein synthesis, as assessed by luciferase activity, was then determined as a percent of vehicle-treated samples. Again, a number of the derivatives showed luciferase activity comparable to that of Congo red, indicating protection from cytotoxicity and cell death.

### EXAMPLE 15

### Identification of Additional Compounds for Use in Decreasing Cell Toxicity or Cell Death, or for Decreasing Aggregates or Inclusions Formed By Amyloidogenic Proteins

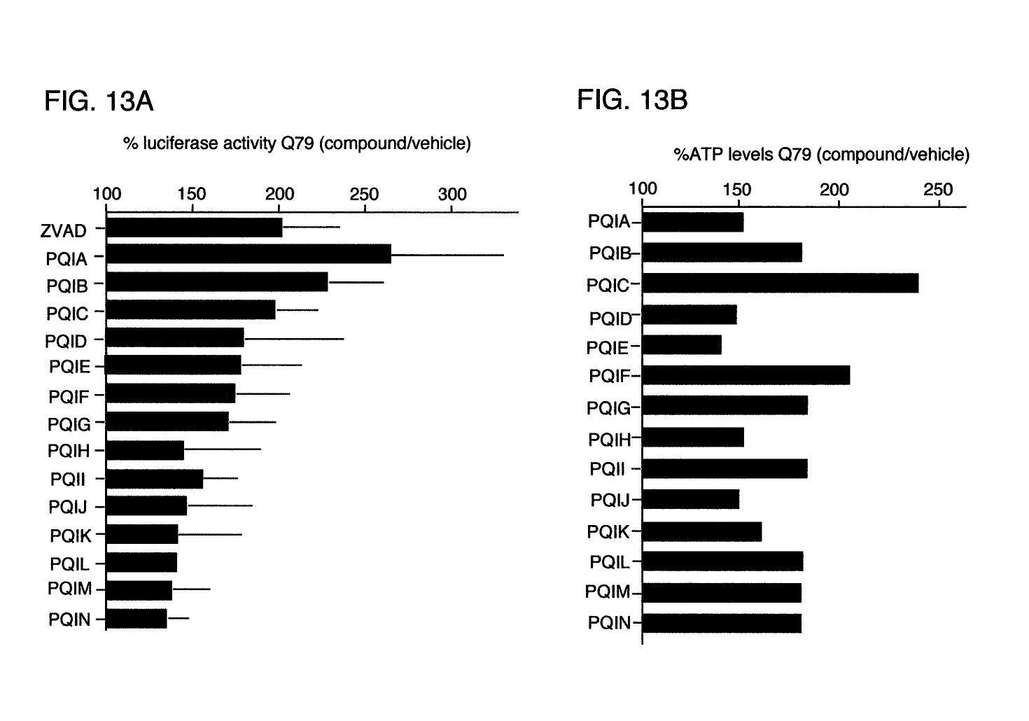

A ChemBridge small molecule library (San Diego, Calif.) was screened for compounds that decrease cell death or toxicity. The screen was carried out in transiently transfected HeLa cells expressing Q79, as described above in Example 14. The effect of the library members on luciferase activity was assayed as described above. FIG. 13A shows the result of compounds from the library (identified as PQIA-PQIN; for polyglutamine inhibitors A-N) that were shown to increase luciferase activity in Q79 expressing cells.

These compounds were also assessed for their ability to protect HeLa cells transiently transfected with Q79-induced from ATP depletion. This high-throughput assay was carried out as described above. As shown in FIG. 13B, compared to vehicle controls, these compounds protected cells from ATP depletion.

The structures and ChemBridge library product numbers for the compounds that tested positively in the ATP depletion assays and luciferase assays are shown in FIGS. 14A-14L. In addition, derivatives of some of the identified compounds, as shown in FIGS. 14A-14F, were identified and tested for their cell protective effects, as assayed by ATP depletion tests and luciferase activity analysis after administration of Q79 transiently transfected HeLa cells with 5 μM or 25μM of the test compound. As shown in FIGS. 14A-14F, PQIA, PQIA-1, PQIA-2, PQIA-3, PQIB, PQIC, PQIC-1, PQID, PQIM, PQID-1, PQID-2, PQIF, PQIF-2, and PQIG were cytoprotective, and can be used to decrease cell death or toxicity in cells expressing amyloidogenic proteins, for example, expanded polyglutamine repeats, or to decrease aggregate or inclusions formed by an amyloidogenic protein. These compounds can also be used to treat a subject with a condition associated with an expressed amyloidogenic protein.

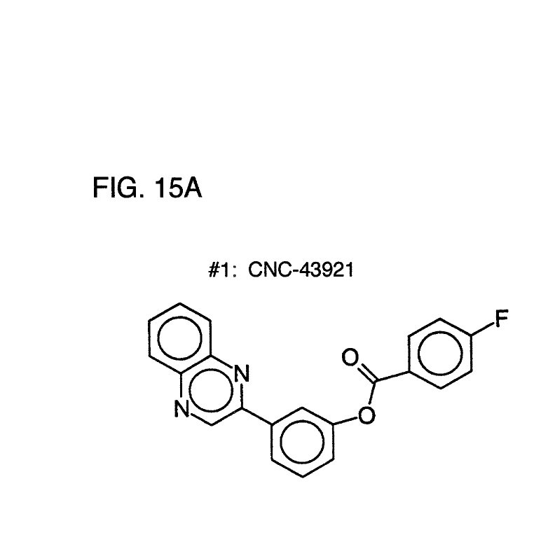

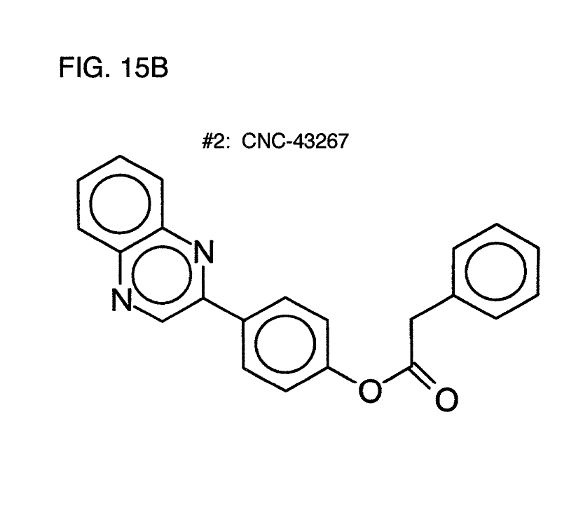

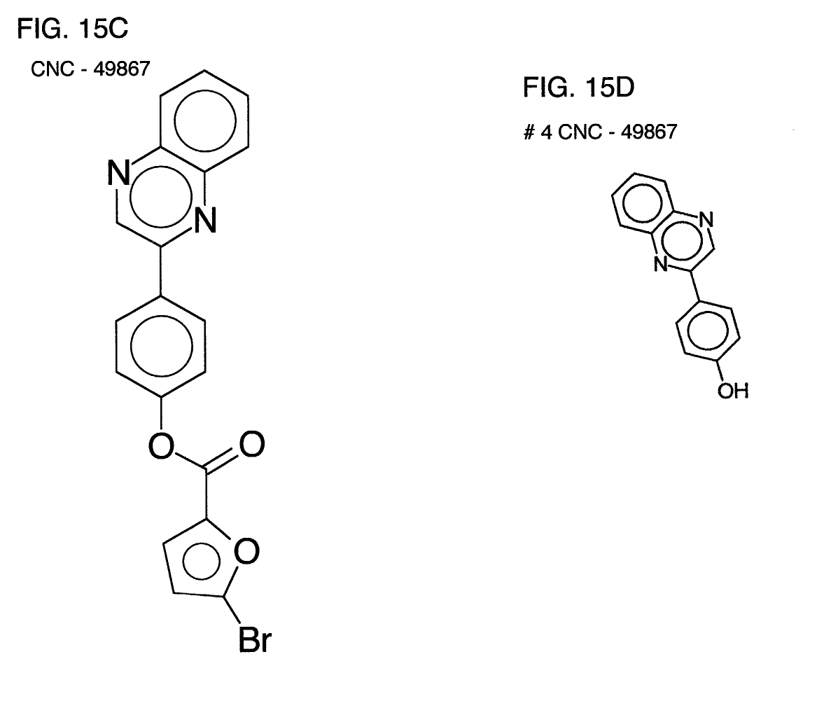

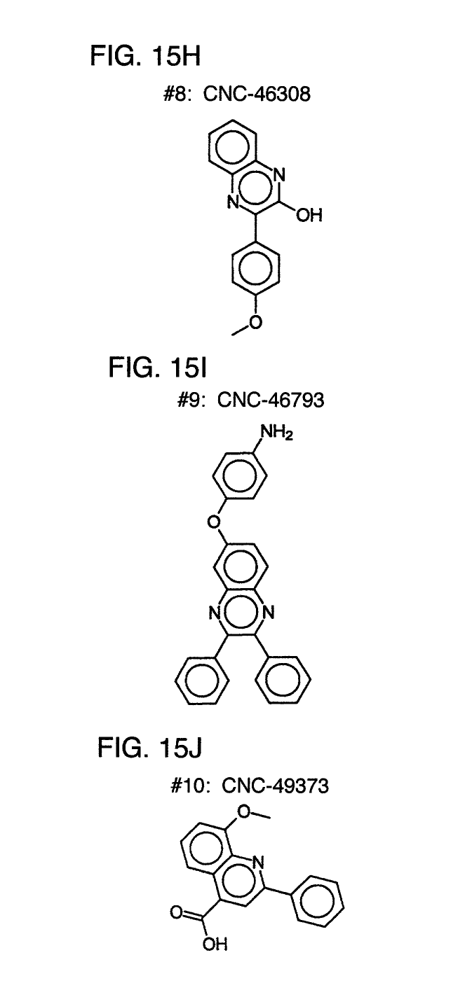

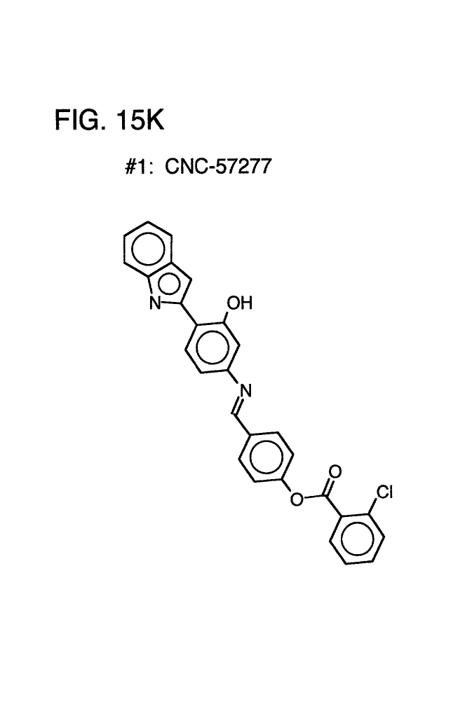

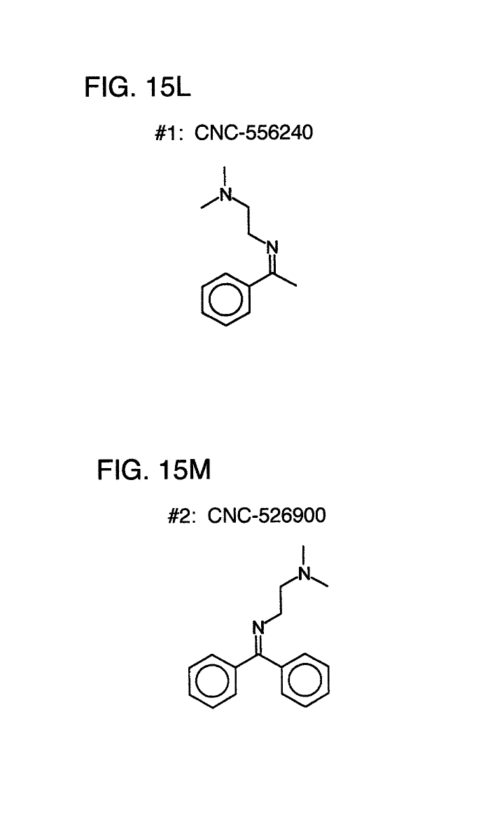

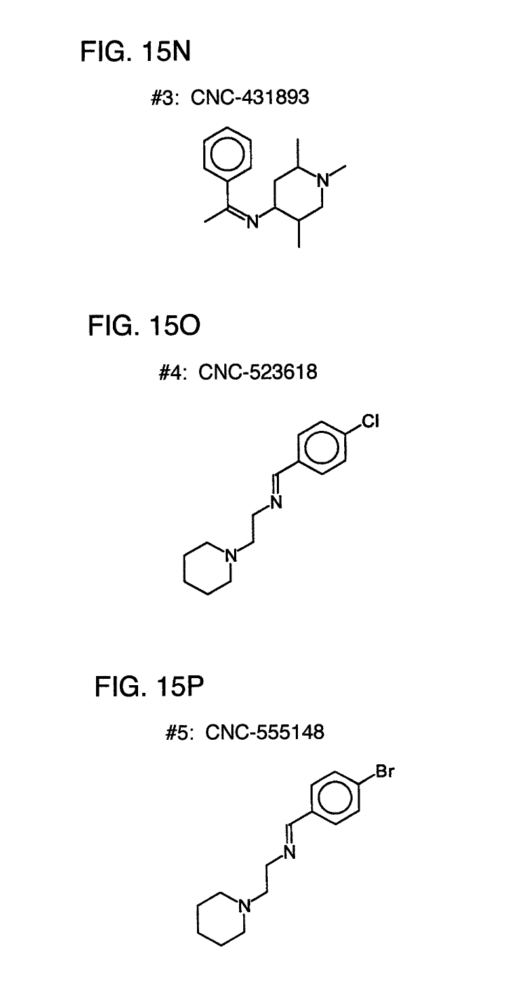

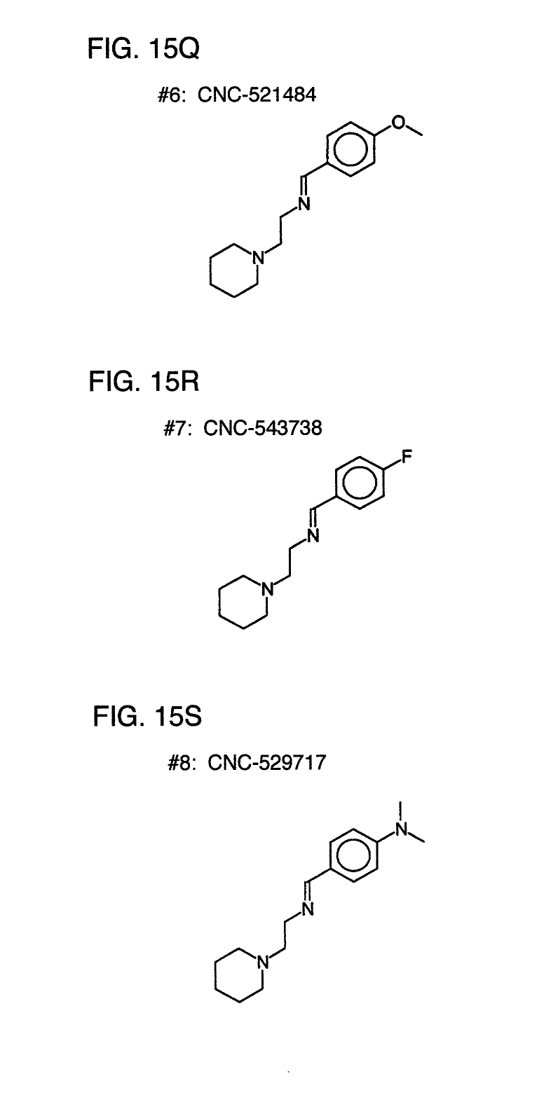

FIGS. 15A-15S show the structures and product numbers of additional derivatives of PQID (FIGS. 15A-15J), PQIA (FIG. 15K), and PQIB (FIGS. 15L-15S). These compounds are commercially available from ChemNavigator (San Diego, Calif.), and can be used to decrease cell death or toxicity in cells expressing amyloidogenic proteins, for example, expanded polyglutamine repeats, or to decrease aggregate or inclusions formed by an amyloidogenic protein. The compounds can also be used to treat conditions associated with an expressed amyloidogenic protein.

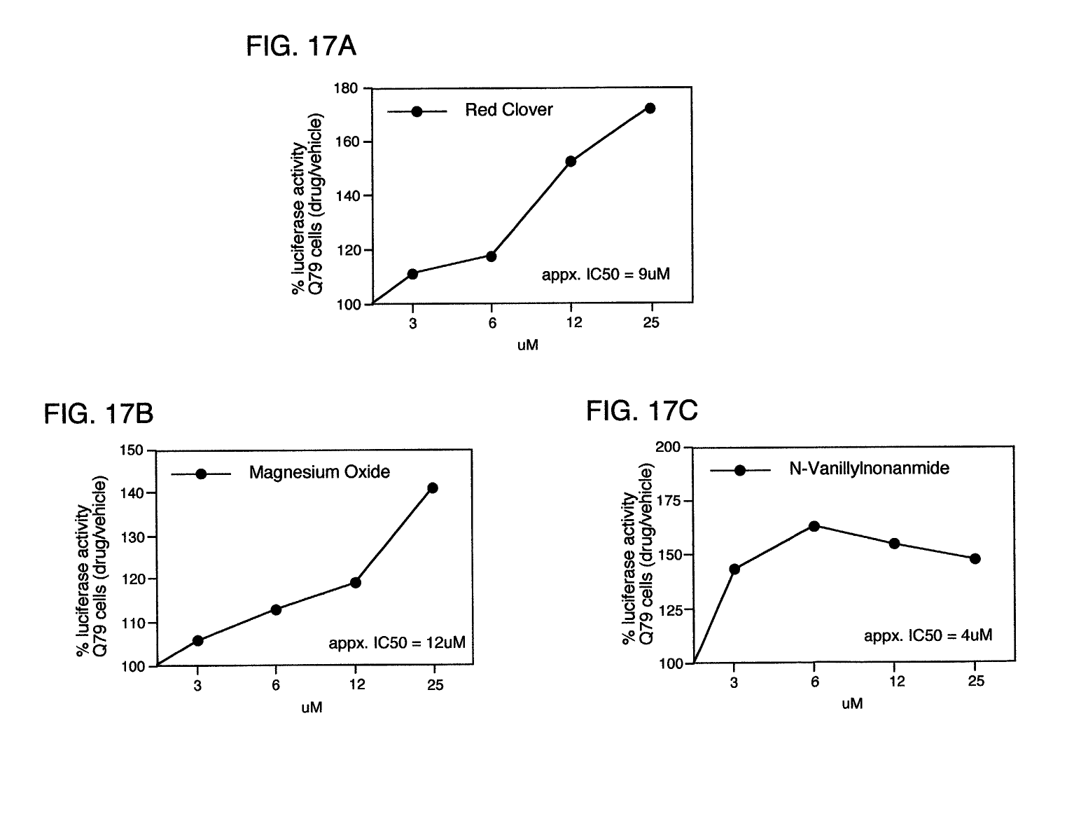

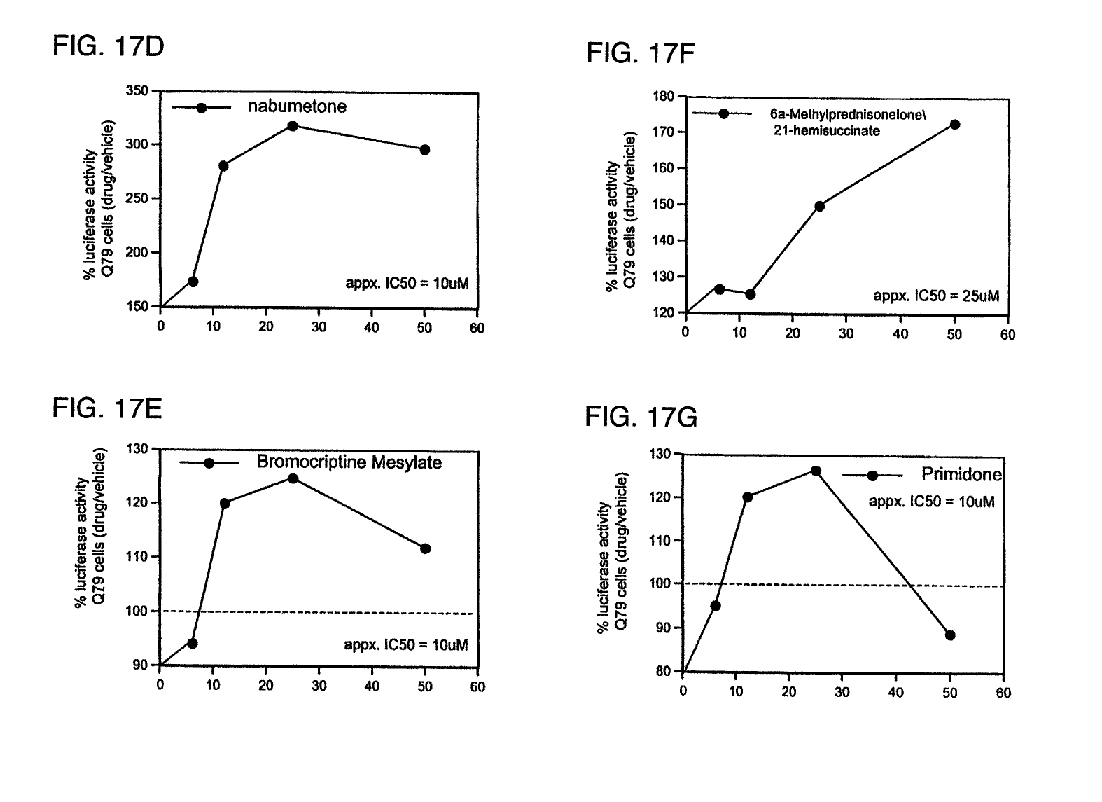

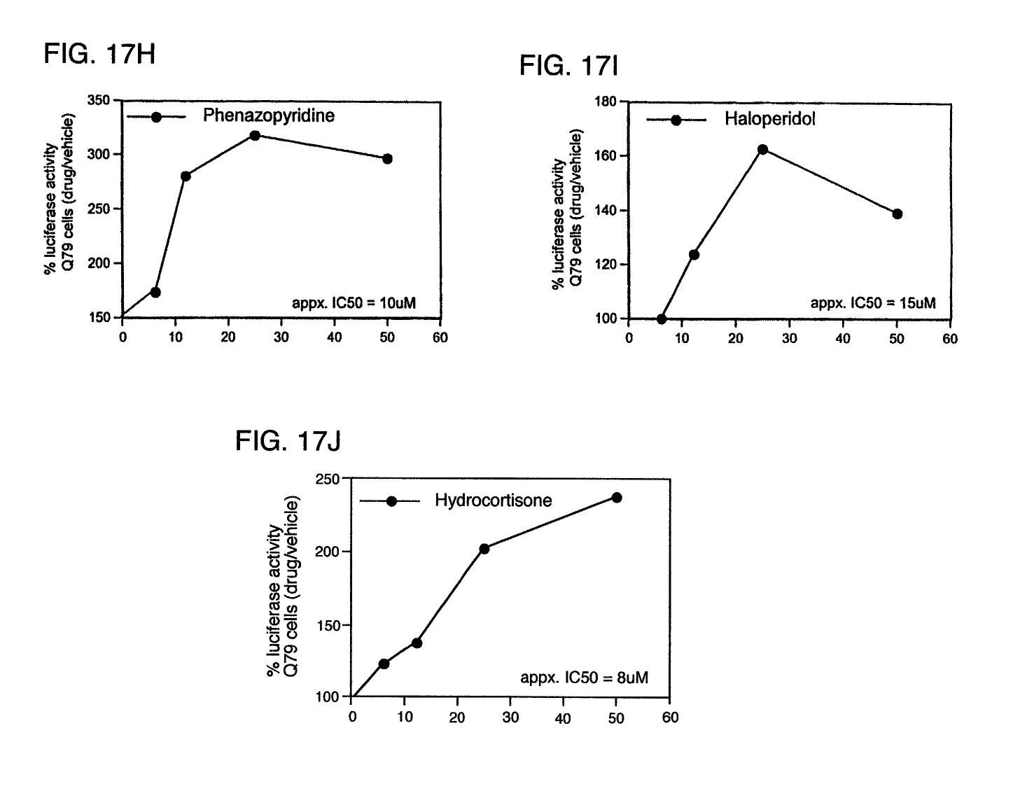

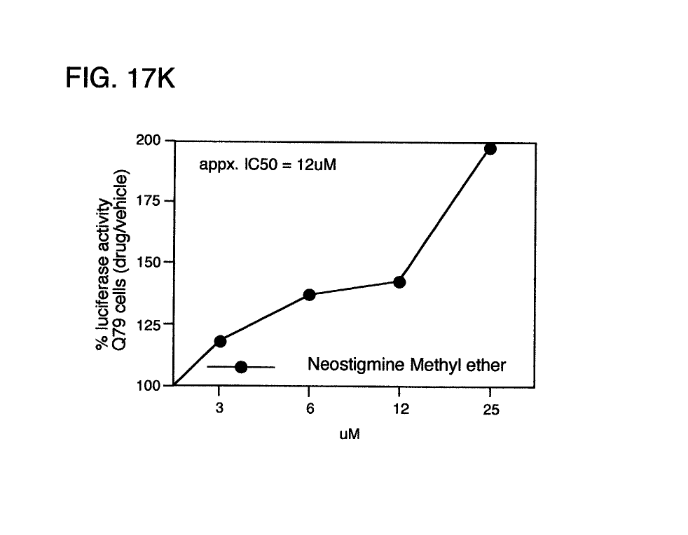

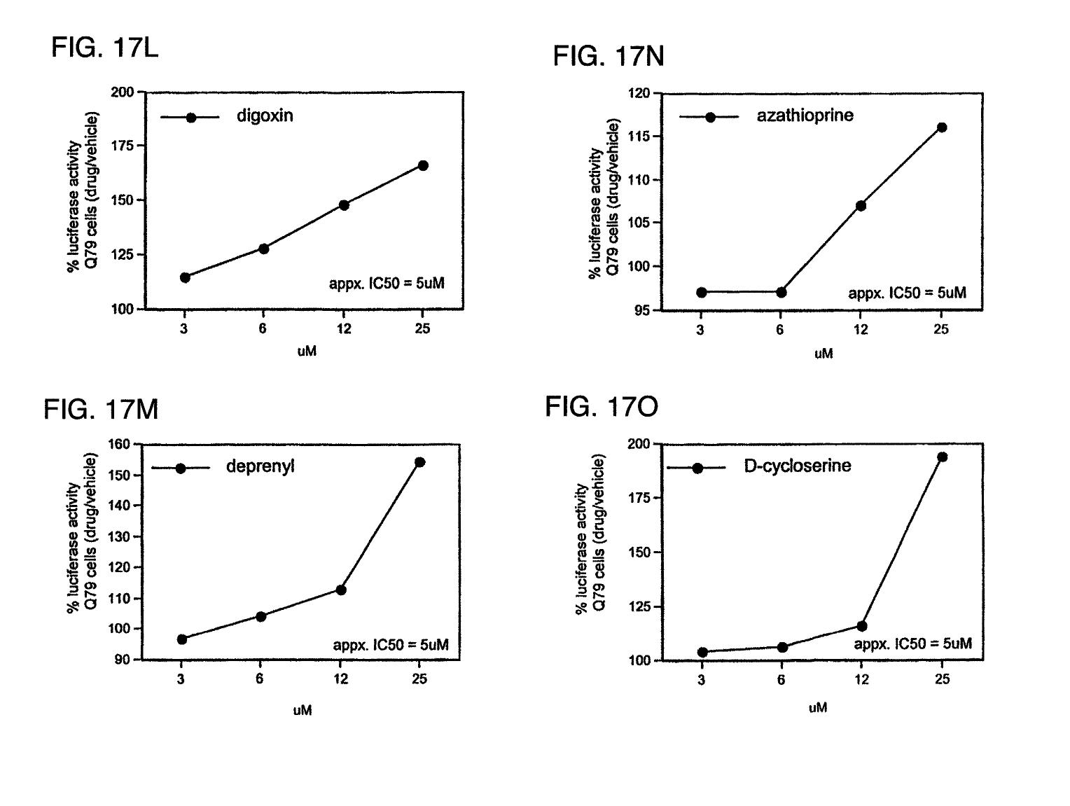

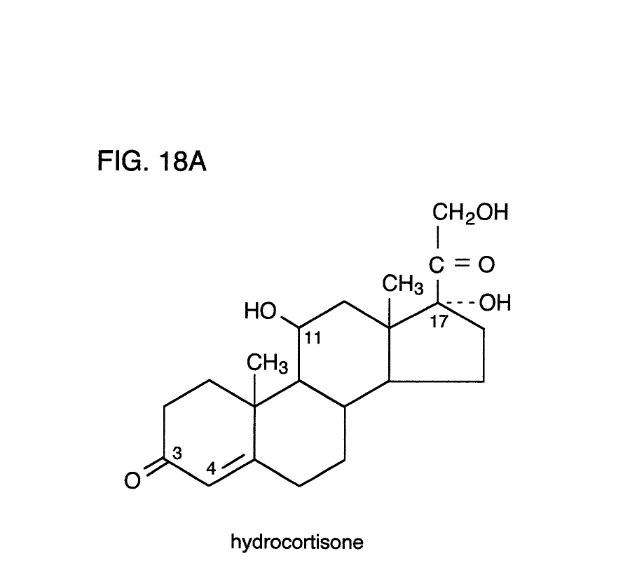

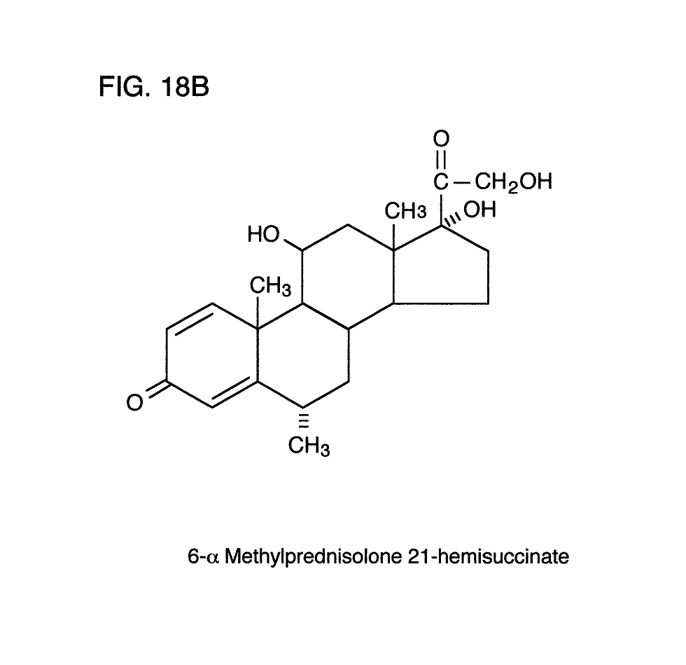

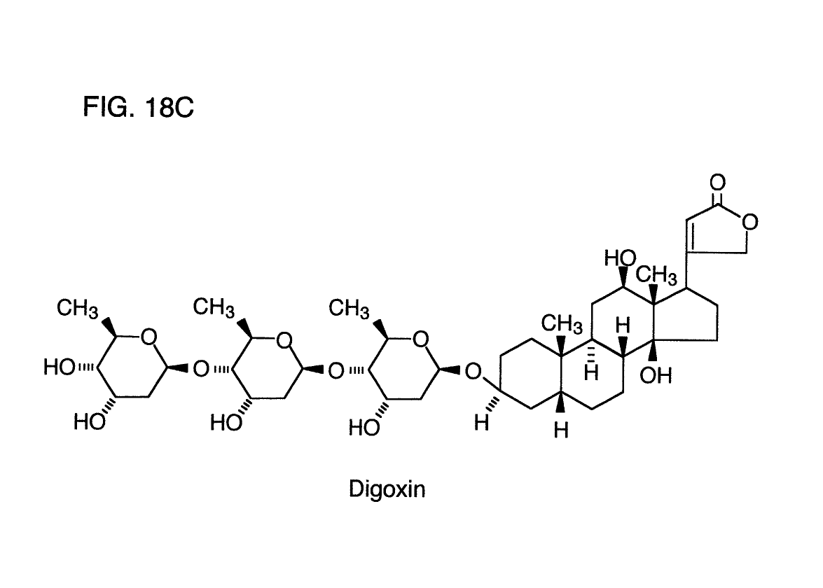

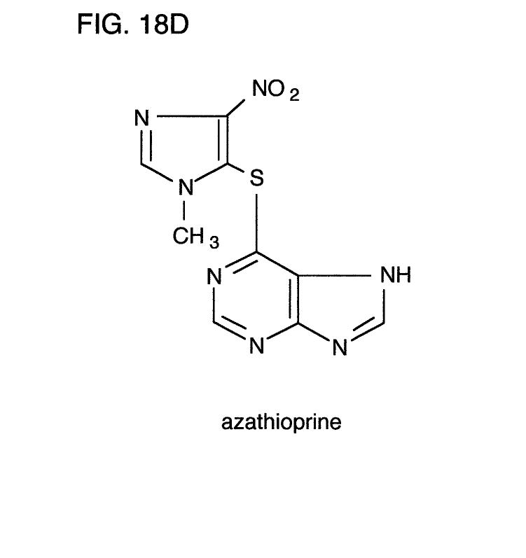

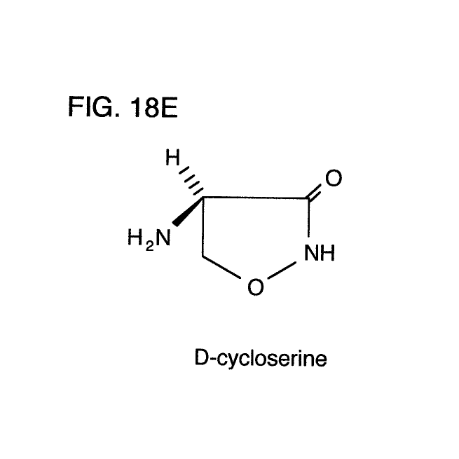

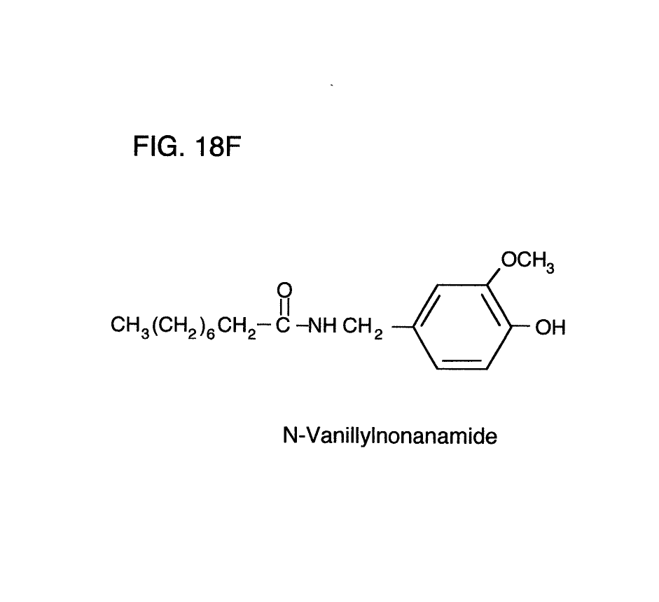

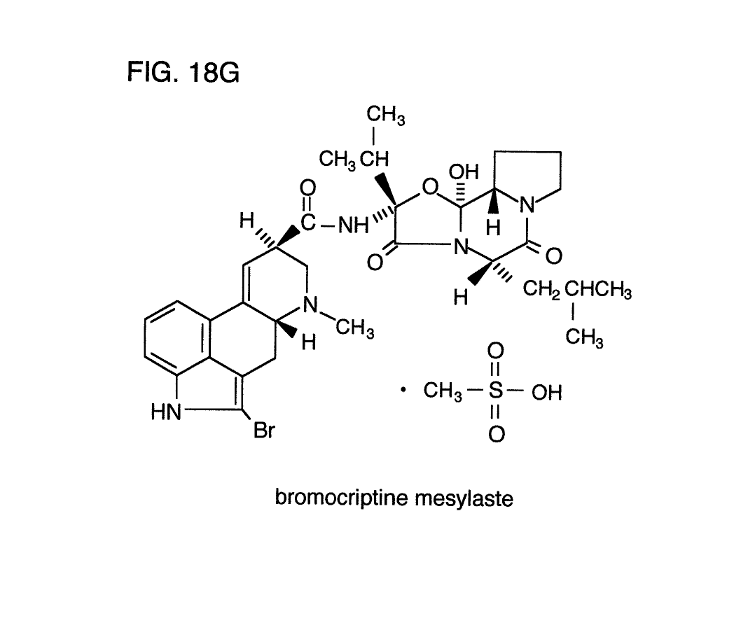

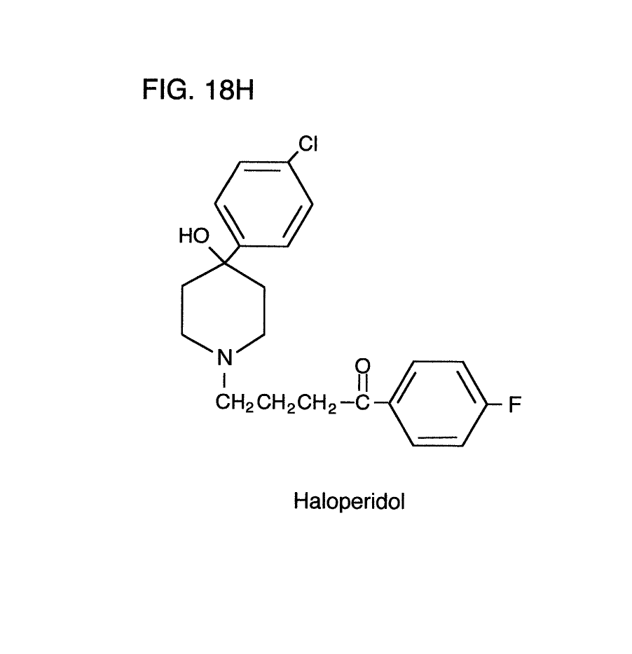

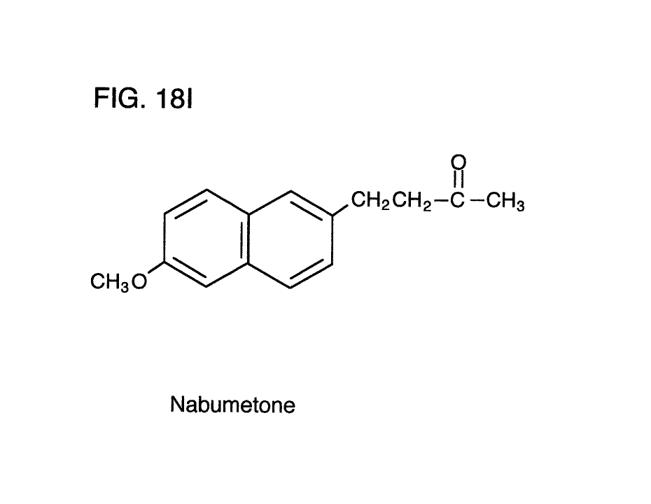

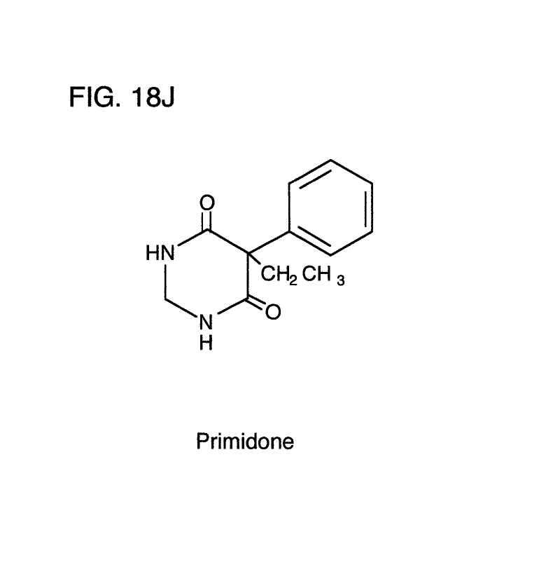

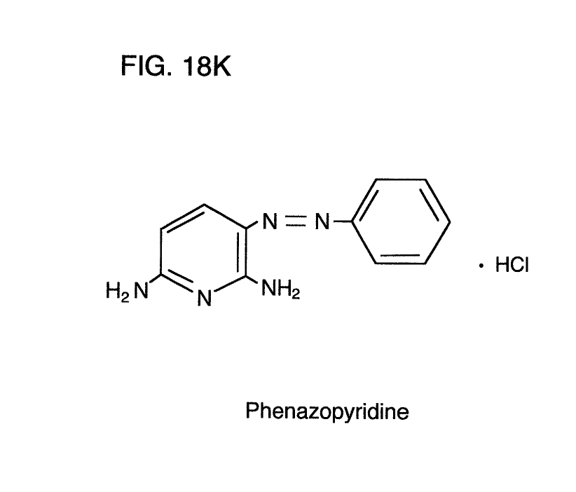

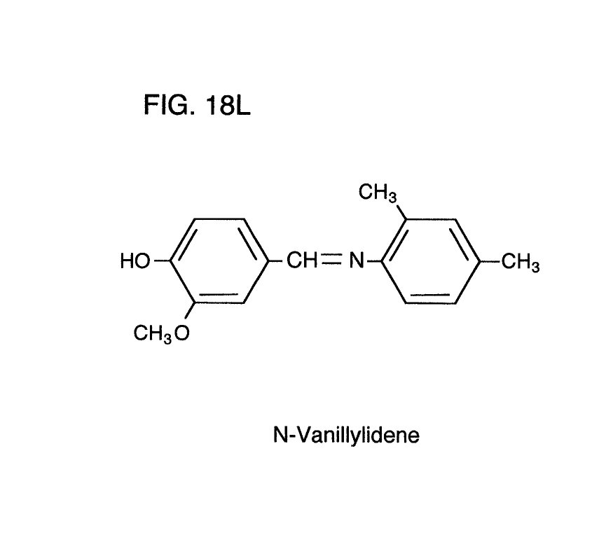

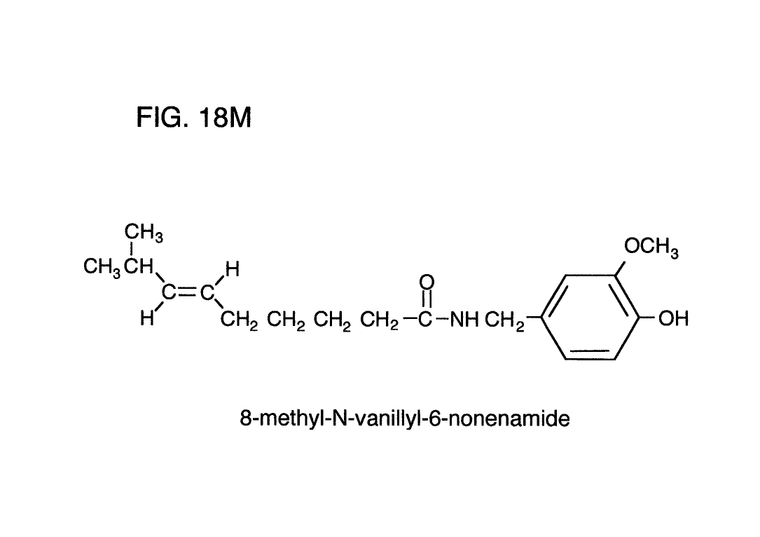

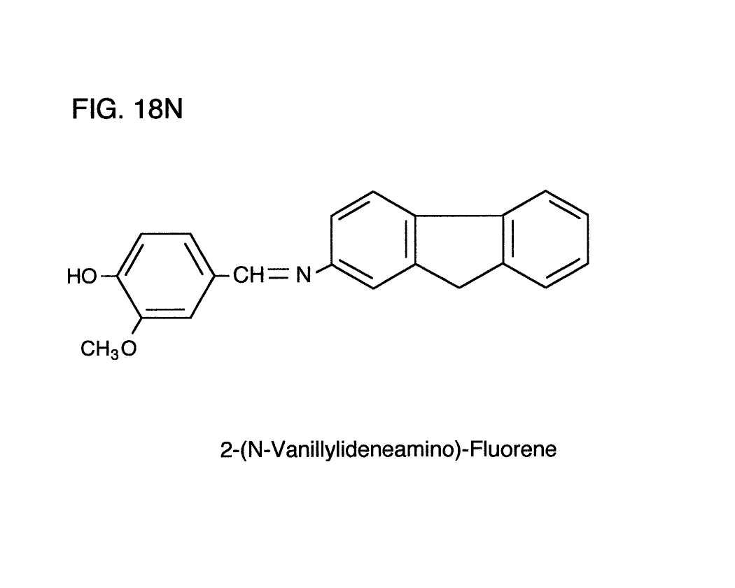

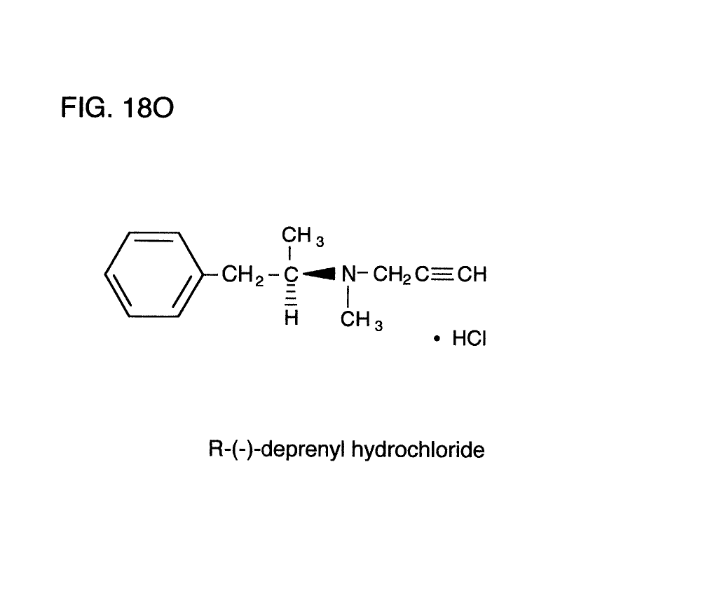

In addition, a panel of FDA-approved drugs and other natural compounds was tested to identify decreased cell death or toxicity. These compounds are available from pharmacies, as well as from the manufacturer (Physician's Desk Reference, 52 Edition, Medical Economics Company, 1998). The screen was carried out as described above in Example 14. The effect of the test compounds on luciferase activity was assayed as described above. FIGS. 17A-17K shows the result of some of the compounds from the library that were shown to increase luciferase activity in Q79 expressing cells. The following compounds were found to protect against inhibition of protein synthesis: bromocriptine mesylate; haloperidol; nabumetone; primidone; hydrocortisone; phenazopyridine; R-(−)-deprenyl hydrochloride; 6a-methylprednisolone 21-hemisuccinate; digoxin; azathioprine; D-cycloserine; red clover; magnesium oxide; N-vanillylnonanmide; and neostigmine methyl ether. The structures of many of the identified compounds, as well as some derivatives of N-vanillylnonanmide are shown in FIGS. 18A-18O. The compounds can be used to decrease cell death or toxicity in cells expressing amyloidogenic proteins, for example, expanded polyglutamine repeats, or to decrease aggregate or inclusions formed by an amyloidogenic protein. These compounds can also be used to tread a subject with a condition associated with an expressed amyloidogenic protein.

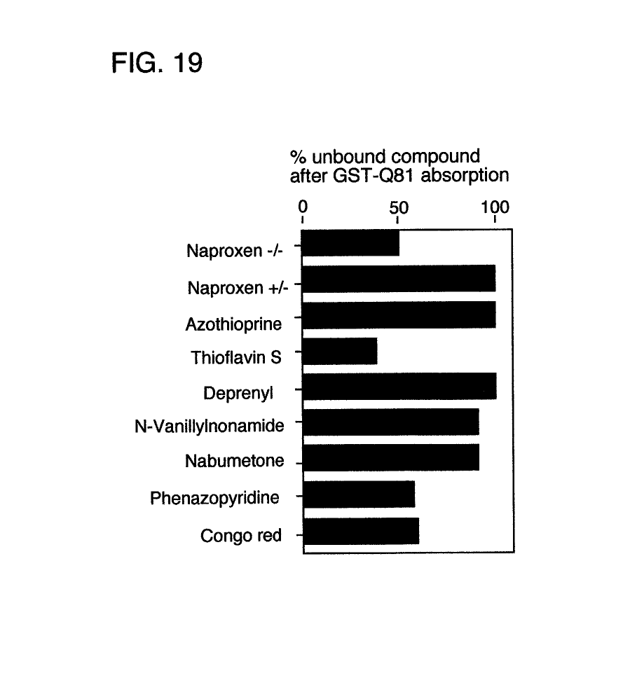

Binding of the above-identified compounds to amyloid-like Q81 aggregates was also assessed by a chemical absorption assay. The compounds indicated in FIG. 19 (25 μM of each) were pre-absorbed with a Q81 GST recombinant protein and the percentage absorbance of the compound remaining in the supernatant after absorption with GST-Q81 beads was then detected. As shown in FIG. 19 some of the compounds interacted with the GST-Q81 beads, while other did not. These results indicate that some compounds may protect cells from cytotoxicity or death by physically interacting with expanded polyglutamine repeats, while other do not physically interact with expanded polyglutamine repeats.

All publications mentioned in this specification are herein incorporated by reference to the same extent as if each independent publication or patent application was specifically and individually indicated to be incorporated by reference.

What is claimed is:

1. A method for decreasing cell death or toxicity, said method comprising the step of contacting a cell or an animal expressing an expanded polyglutamine repeat with diphenyldiazo-bis-alpha-napthylaminesulfonate, or a pharmaceutically effective derivative or salt thereof, in an amount sufficient to decrease said cell death or toxicity.

2. A method for decreasing aggregates or inclusions formed by expanded polyglutamine repeats in a cell or animal, said method comprising the step of contacting a cell or animal expressing an expanded polyglutamine repeat with diphenyldiazo-bis-alpha-napthylaminesulfonate, or a pharmaceutically effective derivative or salt thereof, in an amount sufficient to decrease said aggregates or inclusions.

3. The method of claim 1 or 2, wherein said expanded polyglutamine repeat is resistant to at least one of the compounds chosen from the group consisting of minocycline, daunomycin, rolitetracycline, Chrysamine G, iota-carrageenan, and dextran.

4. A method for decreasing cell death or toxicity, said method comprising the step of contacting a cell or an animal expressing an amyloidogenic protein with any of bromocriptine mesylate; haloperidol; nabumetone; primidone; hydrocortisone; phenazopyridine; R-(−)-deprenyl hydrochloride; 6a-methylprednisolone 21-hemisuccinate; digoxin; azathioprine; D-cycloserine; red clover; magnesium oxide; N-vanillylnonanmide; neostigmine methyl ether; a pharmaceutically effective derivative, salt, or isomer thereof; or a compound having the formula selected from any of: 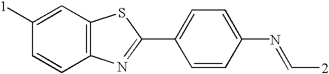
wherein 1 is CH₃ or H, and 2 is 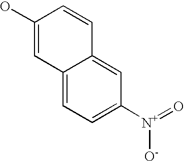
or wherein 1 is CH₃, and 2 is 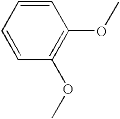
or wherein 1 is CH₃, and 2 is 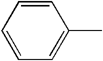
or a pharmaceutically effective derivative, salt, or isomer thereof; 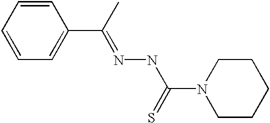
or a pharmaceutically effective derivative, salt, or isomer thereof; 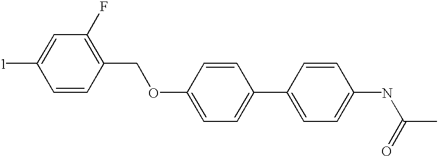
wherein 1 is H or NO₂⁻, or a pharmaceutically effective derivative, salt, or isomer thereof; 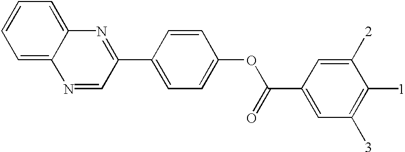
wherein 1 is Cl, and 2 and 3 are H; or wherein 1 and 3 are H, and 2 is NO₂; or wherein 1 is Br, 2 is H, and 3 is NO₂; or wherein 1 is Cl, 2 is H, and 3 is Br, or a pharmaceutically effective derivative, salt, or isomer thereof; 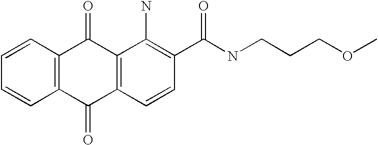
or a pharmaceutically effective derivative, salt, or isomer thereof; 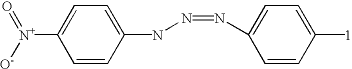
wherein 1 is NO₂, Br, or O₂, or a pharmaceutically effective derivative, salt, or isomer thereof; 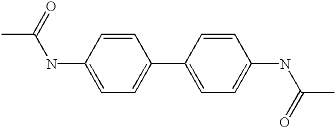
or a pharmaceutically effective derivative, salt, or isomer thereof 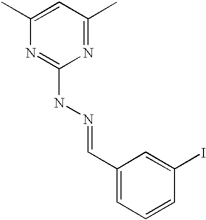
or a pharmaceutically effective derivative, salt, or isomer thereof; 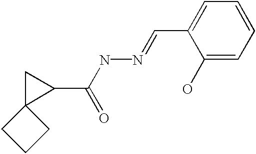
or a pharmaceutically effective derivative, salt, or isomer thereof; 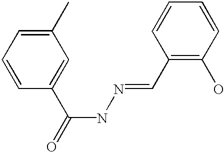
or a pharmaceutically effective derivative, salt, or isomer thereof; 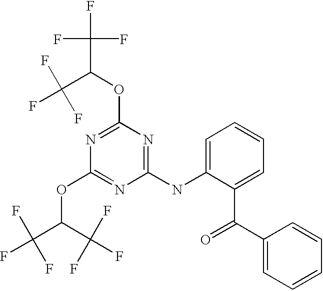
or a pharmaceutically effective derivative, salt, or isomer thereof; or 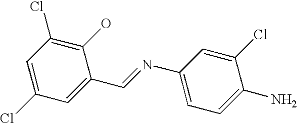
or a pharmaceutically effective derivative, salt, or isomer thereof, in an amount sufficient to decrease said cell death or toxicity, and wherein if said compound is haloperidol, phenazopyridine, or R-(−)-deprenyl, then said amyloidogenic protein is not beta-amyloid.

5. A method for decreasing aggregates or inclusions formed by an amyloidogenic protein in a cell or animal, said method comprising the step of contacting a cell or an animal expressing an amyloidogenic protein with any of bromocriptine mesylate; haloperidol; nabumetone; primidone; hydrocortisone; phenazopyridine; R-(−)-deprenyl hydrochloride; 6a-methylprednisolone 21-hemisuccinate; digoxin; azathioprine; D-cycloserine; red clover; magnesium oxide; N-vanillylnonanmide; neostigmine methyl ether; a pharmaceutically effective derivative, salt, or isomer thereof; or a compound having the formula selected from any of: 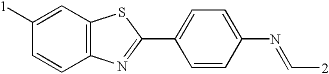
wherein 1 is CH₃ or H, and 2 is 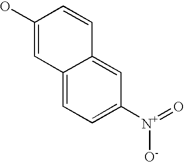
or wherein 1 is CH₃, and 2 is 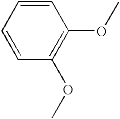
or wherein 1 is CH₃, and 2 is 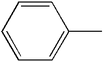
or a pharmaceutically effective derivative, salt, or isomer thereof; 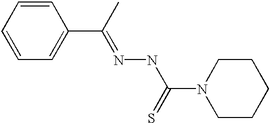
or a pharmaceutically effective derivative, salt, or isomer thereof; 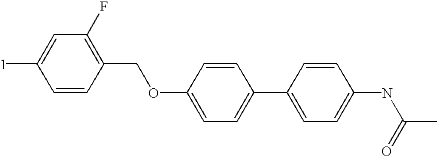
wherein 1 is H or NO₂⁻, or a pharmaceutically effective derivative, salt, or isomer thereof; 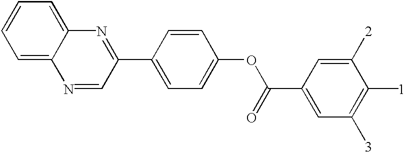
wherein 1 is Cl, and 2 and 3 are H; or wherein 1 and 3 are H, and 2 is NO₂; or wherein 1 is Br, 2 is H, and 3 is NO₂; or wherein 1 is Cl, 2 is H, and 3 is Br, or a pharmaceutically effective derivative, salt, or isomer thereof; 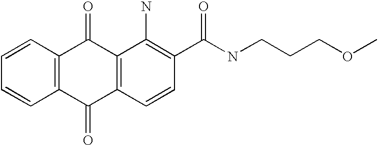
or a pharmaceutically effective derivative, salt, or isomer thereof; 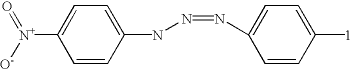
wherein 1 is NO₂, Br, or O₂, or a pharmaceutically effective derivative, salt, or isomer thereof; 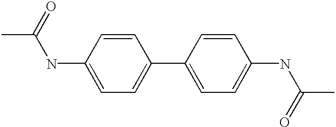
or a pharmaceutically effective derivative, salt, or isomer thereof 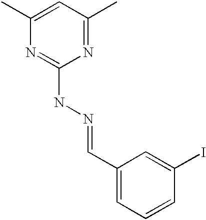
or a pharmaceutically effective derivative, salt, or isomer thereof; 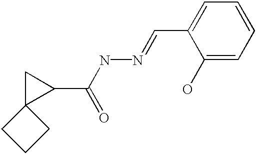
or a pharmaceutically effective derivative, salt, or isomer thereof, 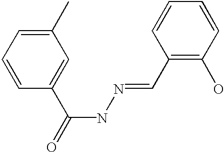
or a pharmaceutically effective derivative, salt, or isomer thereof; 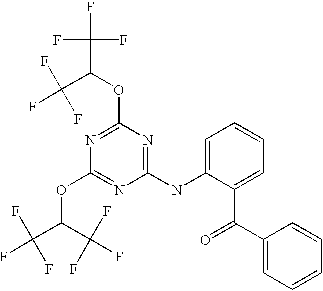
or a pharmaceutically effective derivative, salt, or isomer thereof; or 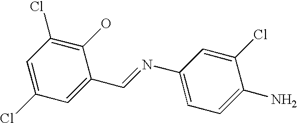
or a pharmaceutically effective derivative salt, or isomer thereof, in an amount sufficient to decrease said aggregates or inclusions, and wherein if said compound is haloperidol, phenazopyridine, or R-(−)-deprenyl, then said amyloidogenic protein is not beta-amyloid.

6. The method claim 1, 2, 4, or 5, wherein said cell is mammalian.

7. The method of claim 6, wherein said cell is human.

8. The method of claim 6, wherein said cell is a rodent cell.

9. The method of claim 6, wherein said cell is a germ-line cell.

10. The method of claim 6, wherein said cell is ex vivo.

11. The method of claim 4 or 5, wherein said amyloidogenic protein is an expanded polyglutamine repeat.

12. A method for treating a condition, or a symptom associated with a condition, in a subject at risk for having an expressed expanded polyglutamine repeat, said method comprising administering diphenyldiazo-bis-alpha-napthylaminesulfonate, or a pharmaceutically effective derivative or salt thereof, to said subject.

13. A method for treating a condition, or a symptom associated with a condition, in a subject at risk for having an expressed amyloidogenic protein, said method comprising administering any of bromocriptine mesylate; haloperidol; nabumetone; primidone, hydrocortisone; phenazopyridine; R-(−)-deprenyl hydrochloride; 6a-methylprednisolone 21-hemisuccinate; digoxin; azathioprine; D-cycloserine; red clover; magnesium oxide; N-vanillylnonanmide; neostigmine methyl ether; a derivative, salt, or isomer thereof; or a compound having the formula selected from the any of: 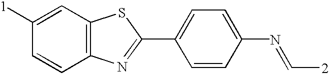
wherein 1 is CH₃ or H, and 2 is 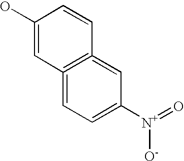
or wherein 1 is CH₃, and 2 is 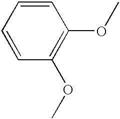
or wherein 1 is CH₃, and 2 is 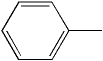
or a pharmaceutically effective derivative, salt, or isomer thereof; 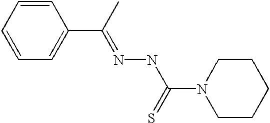
or a pharmaceutically effective derivative, salt, or isomer thereof; 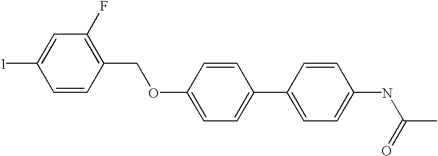
wherein 1 is H or NO₂⁻, or a pharmaceutically effective derivative, salt, or isomer thereof; 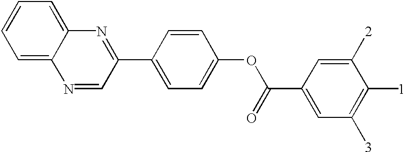
wherein 1 is Cl, and 2 and 3 are H; or wherein 1 and 3 are H, and 2 is NO₂; or wherein 1 is Br, 2 is H, and 3 is NO₂; or wherein 1 is Cl, 2 is H, and 3 is Br, or a pharmaceutically effective derivative, salt, or isomer thereof; 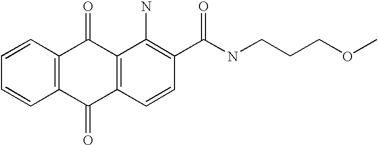
or a pharmaceutically effective derivative, salt, or isomer thereof; 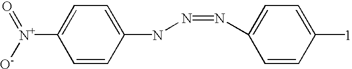
wherein 1 is NO₂, Br, or O₂, or a pharmaceutically effective derivative, salt, or isomer thereof; 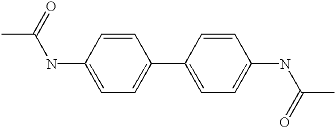
or a pharmaceutically effective derivative, salt, or isomer thereof 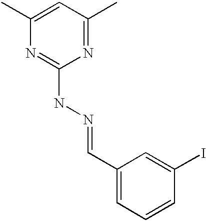
or a pharmaceutically effective derivative, salt, or isomer thereof; 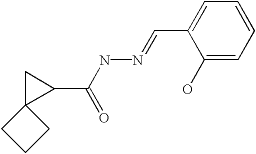
or a pharmaceutically effective derivative, salt, or isomer thereof; 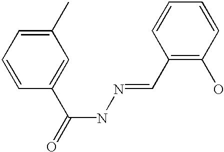
or a pharmaceutically effective derivative, salt, or isomer thereof; 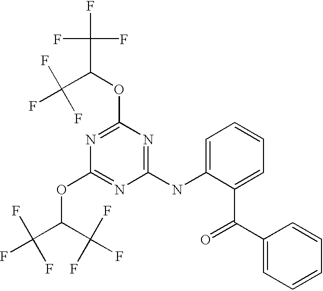
or a pharmaceutically effective derivative, salt, or isomer thereof; or 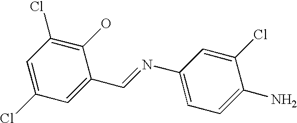
or a pharmaceutically effective derivative, salt, or isomer thereof to said subject.

14. The method of claim 12 or 13, wherein said condition is a neurodegenerative disease.

15. The method of claim 14, wherein said neurodegenerative disease is any of Huntington's disease, spinobulbar muscular atrophy (SBMA), spino-cerebellar ataxia type 1, spino-cerebellar ataxia type 2, spino-cerebellar ataxia type 3, spino-cerebellar ataxia type 6, spino-cerebellar ataxia type 7, dentatorubral-pallidoluysian atrophy, or familial schizophrenia.

16. The method of claim 12 or 13, wherein said condition is male infertility.

17. The method of claim 13, wherein said condition is caused by an amyloidogenic protein.

18. The method of claim 12 or 17, wherein said condition is caused by expanded polyglutamine repeats.

19. The method of claim 12 or 13, wherein said subject is a mammal.

20. The method of claim 18, wherein said subject is a human.

21. The method of claim 12, wherein said expressed expanded polyglutamine repeat is resistant to at least one of the compounds chosen from the group consisting of minocycline, daunomycin, rolitetracycline, Chrysamine G, iota-carrageenan, or dextran.

22. The method of any of claims 1, 2, or 12, wherein said derivative is any one of Direct Orange 8, Direct Yellow 26, Direct Yellow 28, Direct Blue 158, Direct Orange 6, Direct Red 1, Direct Orange 1, or Direct Black 51.

23. The method of any of claims 1, 2, 4, or 5, wherein said animals is an animal diagnosed with, or having an increased likelihood of developing a neurodegenerative disease.

24. The method of claim 23, wherein said neurodegenerative disease is any of Huntington's disease, spinobulbar muscular atrophy (SBMA), spino-cerebellar ataxia type 1, spino-cerebellar ataxia type 2, spino-cerebellar ataxia type 3, spino-cerebellar ataxia type 6, spino-cerebellar ataxia type 7, dentatorubral-pallidoluysian atrophy, or familial schizophrenia.
